(19) United States
(12) Patent Application Publication
(10) Pub. No.: US 2026/0365652 A1
(43) Pub. Date: Nov. 19, 2026

(54) PROCESS FOR PREPARING OPTICALLY-ACTIVE AZABICYCLO RING DERIVATIVE, AND INTERMEDIATE FOR ITS PRODUCTION

(71) Applicants: Kiichi KURODA, Osaka-shi (JP); Kazuki Azuma, Osaka-shi (JP); Rieko Kamei, Osaka-shi (JP); Nayoa Ito, Osaka-shi (JP); Christopher Katz, Marlborough, MA (US); Jalal Haddad, San Diego, CA (US); Quanfang Hu, Chongqing (CN); Peng Yang, Chongaing (CN)

(72) Inventors: Kiichi KURODA, Osaka-shi (JP); Kazuki Azuma, Osaka-shi (JP); Rieko Kamei, Osaka-shi (JP); Nayoa Ito, Osaka-shi (JP); Christopher Katz, Marlborough, MA (US); Jalal Haddad, San Diego, CA (US); Quanfang Hu, Chongqing (CN); Peng Yang, Chongaing (CN)

(21) Appl. No.: 19/633,657

(22) Filed: Mar. 30, 2026

Related U.S. Application Data
(63) Continuation-in-part of application No. 19/504,149, filed on Jul. 26, 2024.
(60) Provisional application No. 63/515,913, filed on Jul. 27, 2023.
(60) Provisional application No. 63/673,376, filed on Jul. 19, 2024.

(51) Int. Cl. C07D 471/08; C07D 519/00

(52) U.S. Cl. CPC C07D 471/08; C07D 519/00

(57) ABSTRACT

The present invention relates to a process for preparing an optically-active azabicyclo ring derivative useful as a pharmaceutical compound, an intermediate for its production, and a process for preparing the intermediate.

### TECHNICAL FIELD

The present disclosure relates to a process for preparing an optically-active azabicyclo ring derivative useful as a pharmaceutical compound, an intermediate for its production, and a process for preparing the intermediate.

### BACKGROUND ART

A process for preparing an optically-active azabicyclo ring derivative, and a process for preparing its related substance have been already known, for example, as a process described in Patent literature 1.

### CITATION LIST

### Patent Literature

[Patent literature 1] WO 2020/045334

### SUMMARY OF INVENTION

### Technical Problem

The purpose of the present disclosure is to provide a process for preparing an optically-active azabicyclo ring derivative useful as a pharmaceutical compound.

### Solution to Problem

The present disclosure provides an excellent industrial process for manufacturing an optically-active azabicyclo ring derivative of formula (12), in a small number of steps, simply, in high yield, and at low cost.

The present disclosure provides an excellent industrial process for manufacturing an optically-active azabicyclo ring derivative of formula (8), in a small number of steps, simply, in high yield, and at low cost.

In an embodiment, more specifically, intermediate (2) is prepared by reacting an inexpensive benzylamine derivative (1) with an inexpensive aldehyde (1a) (Step A). The obtained intermediate (2) is reacted with 1,3-cyclohexadiene to obtain intermediate (3) stereoselectively (Step B). The obtained intermediate (3) is regioselectively and stereoselectively hydroxylated via a hydroboration-oxidation sequence to obtain intermediate (4) (Step C), followed by deprotection of the amino group to prepare intermediate (5) (Step D). The ester group in intermediate (5) is hydrolyzed to obtain intermediate (6) (Step E), and the amino group in the obtained intermediate is protected to prepare intermediate (7) (Step F). The hydroxyl group in the obtained intermediate (7) is oxidized to prepare intermediate (8) simply, in high yield, and at low cost (Step G). Intermediate (8) and amine (8a) are reacted to obtain intermediate (9) (Step H), the carbonyl group in intermediate (9) is converted to a vinylidene group to obtain intermediate (10) (Step I), followed by deprotection of the amino group in intermediate (10) to prepare intermediate (11) (Step J). Finally, the obtained intermediate (11) is reacted with compound (11a) to prepare optically-active azabicyclo ring derivative (12). The found process of (Process 1) can prepare derivative (12), in small number of steps, simply, in high yield, and at low cost.

In an embodiment, the present disclosure provides a process for preparing intermediate (8) simply and in high yield, by oxidizing the hydroxyl group in intermediate (7) to obtain intermediate (8) (Step G), and further provides an additional process for preparing an optically-active azabicyclo ring derivative (12) in high yield from intermediate (8) in only 4 steps (Process 1).

In an embodiment, more specifically, intermediate (2) is prepared by reacting an inexpensive benzylamine derivative (1) with an inexpensive aldehyde (1a) (Step A). The obtained intermediate (2) is converted to intermediate (13) in high yield by reacting intermediate (2) with a 1,3-cyclohexadiene derivative (2a) (Step M). The obtained intermediate (13) is deprotected to obtain intermediate (14) (Step N), followed by an additional deprotection to afford intermediate (15) and newly protecting the amino group to prepare intermediate (16) (Steps O and P). Subsequently, the ester group in intermediate (16) is hydrolyzed to obtain intermediate (8) simply, in high yield, and at low cost (Step Q). Intermediate (8) and amine (8a) are reacted to obtain intermediate (9) (Step H). Further, the carbonyl group in intermediate (9) is converted to a vinylidene group to obtain intermediate (10) (Step I), followed by deprotection of the amino group in intermediate (10) to prepare intermediate (11) (Step J). Finally, the obtained intermediate (11) is reacted with compound (11a) to prepare optically-active azabicyclo ring derivative (12). The found process of (Process 2) can prepare derivative (12), in small number of steps, simply, in high yield, and at low cost.

In an embodiment, the present disclosure provides a process for preparing intermediate (8) simply and in high yield, through the step of reacting intermediate (2) and 1,3-cyclohexadiene derivative (2a) to prepare intermediate (13) (Step M), and further provides an additional process for preparing an optically-active azabicyclo ring derivative (12) in high yield from intermediate (8) in only 4 steps (Process 2).

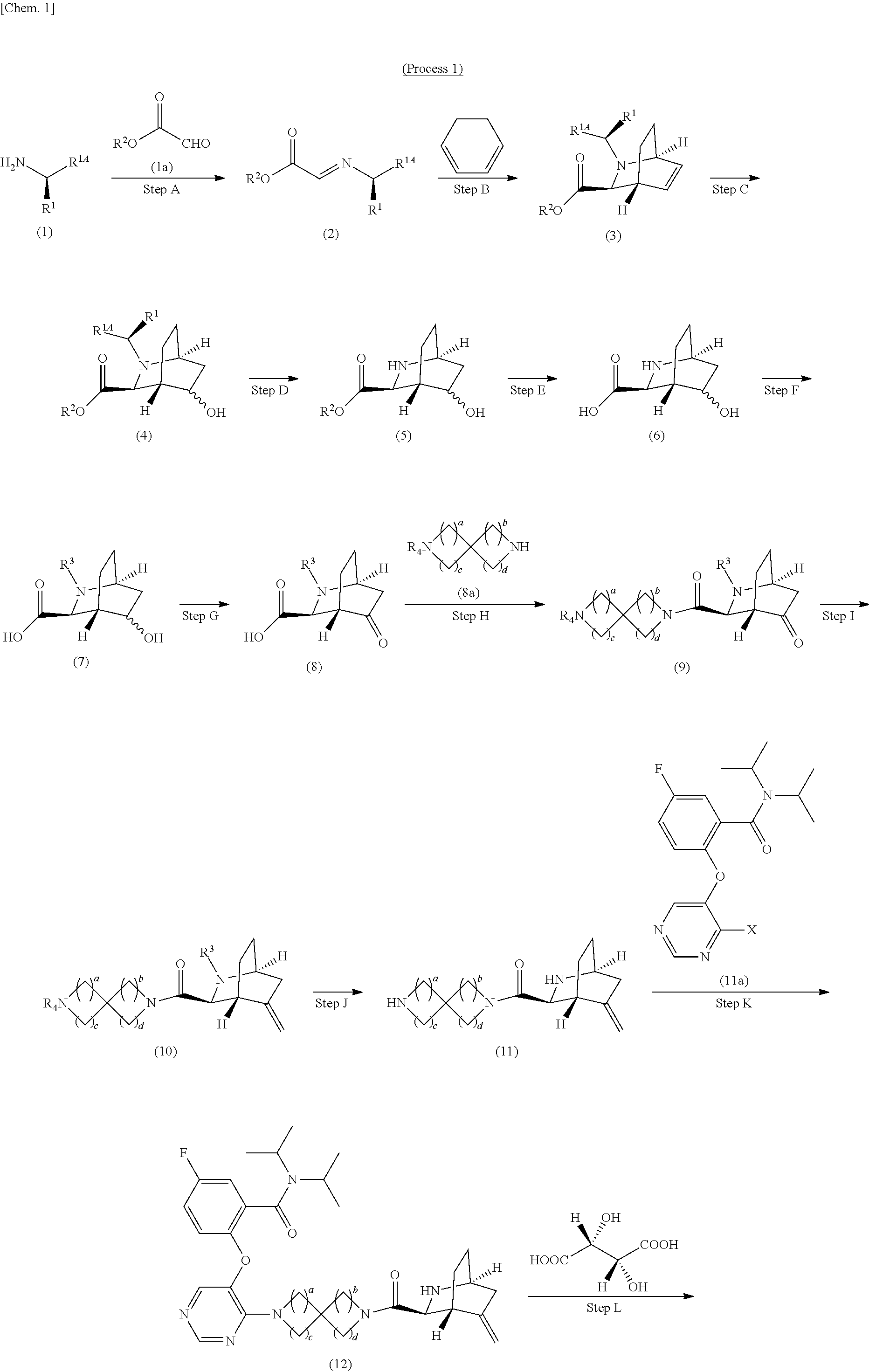

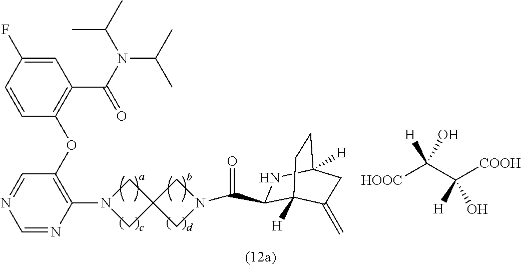

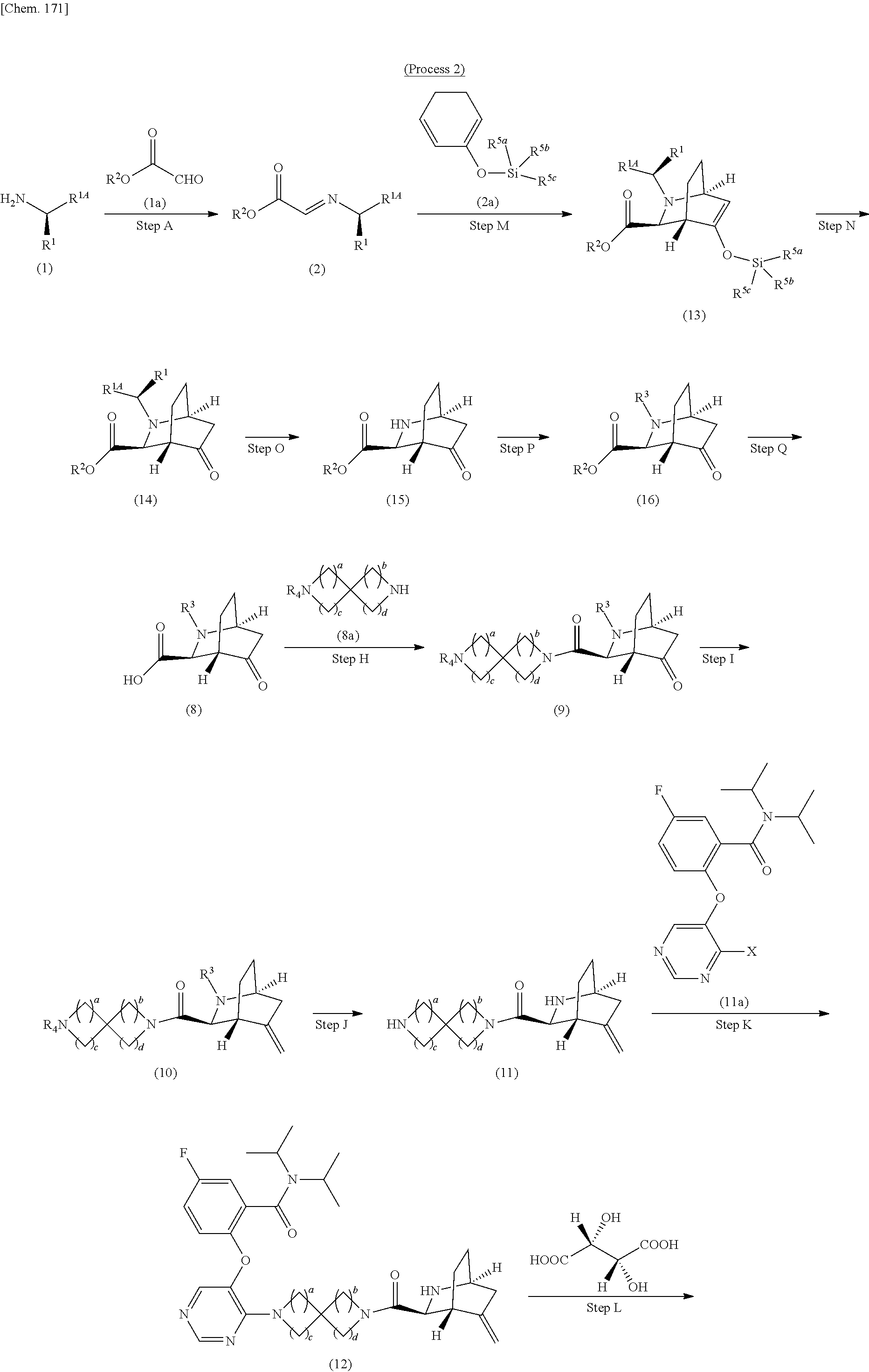

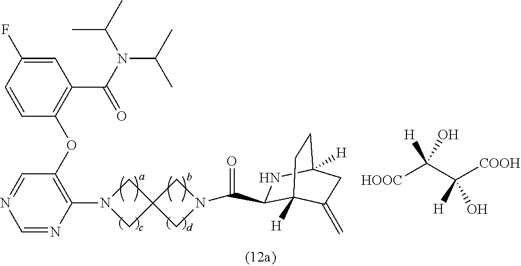

That is, the present disclosure typically provides the followings.

### Item 1

A process for preparing a compound of formula (12):

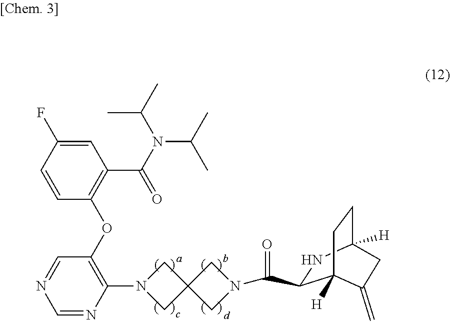

wherein a, b, c, and d are independently 1 or 2,
or a pharmaceutically acceptable salt thereof, or a solvate thereof, comprising Step G shown below,

### Step G:

a compound of formula (7):

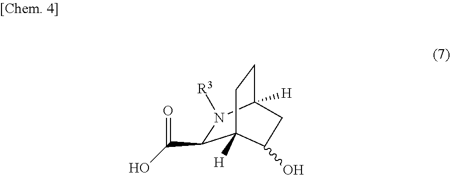

wherein R³ is a protecting group for an amino group, or a pharmaceutically acceptable salt thereof, or a solvate thereof is reacted with an oxidation agent in the presence of a solvent to prepare a compound of formula (8):

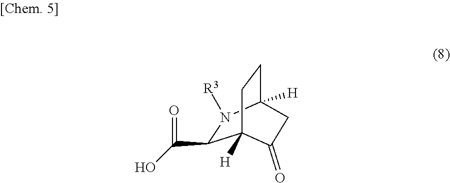

wherein R³ is as defined above,
or a pharmaceutically acceptable salt thereof, or a solvate thereof.

### Item 2

The process of Item 1, wherein the solvent in Step G comprises an ester solvent and/or a halogen solvent.

### Item 3

The process of Item 1, wherein the solvent in Step G comprises an ester solvent.

### Item 4

The process of Item 1, wherein the solvent in Step G comprises ethyl acetate.

### Item 5

The process of any one of Items 1 to 4, wherein the reaction temperature in Step G is −20 °C to 50 °C.

### Item 6

The process of any one of Items 1 to 5, wherein the reaction temperature in Step G is 0 °C to 30 °C.

### Item 7

The process of any one of Items 1 to 6, wherein the oxidation agent used in Step G is 1-methyl-2-azaadamantane-N-oxyl, 2-hydroxy-2-azaadamantane, 9-azanoradamantan-N-oxyl, 1,1,1-triacetoxy-1,1-dihydro-1,2-benziodoxol-3-(1H)-one, potassium 2-iodo-5-methylbenzenesulfonate, “a combination of DMSO and oxalyl chloride”, acetic anhydride, sulfur trioxide-pyridine-complex, N,N′-dicyclohexylcarbodiimide, chromium trioxide, tetrapropylammonium perruthenate, sodium hypochlorite pentahydrate, or 2,2,6,6-tetramethylpiperidine 1-oxyl.

### Item 8

The process of any one of Items 1 to 6, wherein the oxidation agent used in Step G is 2,2,6,6-tetramethylpiperidine 1-oxyl.

### Item 9

The process of any one of Items 1 to 8, which further comprises Step H shown below,

### Step H:

a compound of formula (8):

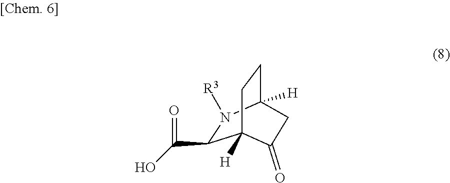

wherein R³ is as defined above,
or a pharmaceutically acceptable salt thereof, or a solvate thereof is reacted with a compound of formula (8a):

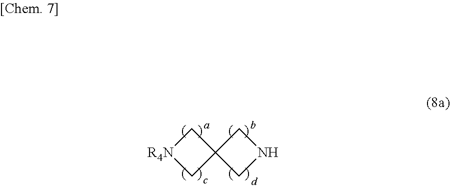

wherein R⁴, a, b, c, and d are as defined above,
or a pharmaceutically acceptable salt thereof, or a solvate thereof in the presence of a solvent and a condensation agent to prepare a compound of formula (9):

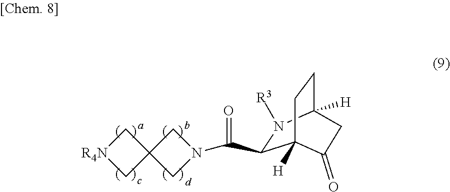

wherein R³, R⁴, a, b, c, and d are as defined above,
or a pharmaceutically acceptable salt thereof, or a solvate thereof.

### Item 10

The process of Item 9, which further comprises Step I shown below,

### Step I:

a compound of formula (9)

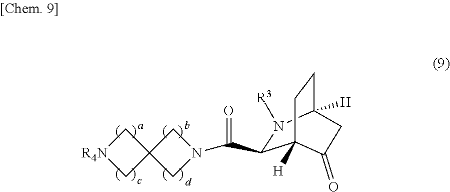

wherein R³, R⁴, a, b, c, and d are as defined above,
or a pharmaceutically acceptable salt thereof, or a solvate thereof is reacted with halogenated methyltriphenylphosphonium, trialkylsilylmethyl anion, or methylsulfone derivative, in the presence of a solvent and a base to prepare a compound of formula (10):

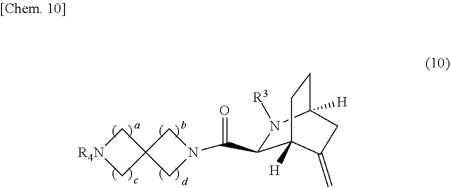

wherein R³, R⁴, a, b, c, and d are as defined above,
or a pharmaceutically acceptable salt thereof, or a solvate thereof.

### Item 11

The process of Item 10, which further comprises Step J shown below,

### Step J:

a compound of formula (10):

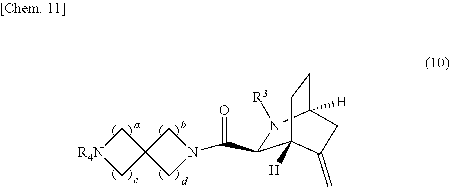

wherein R³, R⁴, a, b, c, and d are as defined above,
or a pharmaceutically acceptable salt thereof, or a solvate thereof is reacted with an acid to prepare a compound of formula (11):

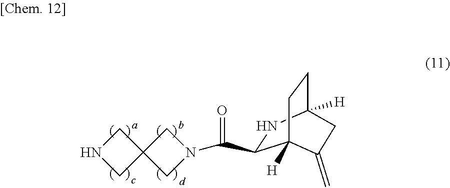

wherein a, b, c, and d are as defined above,
or a pharmaceutically acceptable salt thereof, or a solvate thereof.

### Item 12

The process of Item 11, which further comprises Step K shown below,

### Step K:

a compound of formula (11):

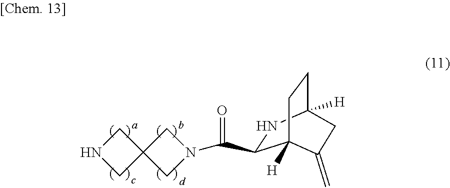

wherein a, b, c, and d are as defined above,
or a pharmaceutically acceptable salt thereof, or a solvate thereof is reacted with a compound of formula (11a):

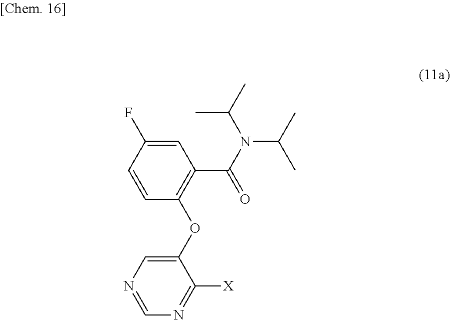

wherein X is fluorine, chlorine, bromine, iodine, p-toluenesulfonyl group, or methanesulfonyl group,
or a pharmaceutically acceptable salt thereof, or a solvate thereof in the presence of a solvent and a base to prepare a compound of formula (12):

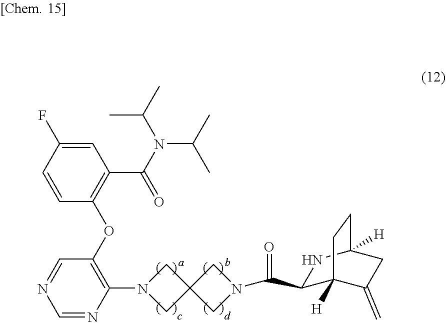

wherein a, b, c, and d are as defined above,
or a pharmaceutically acceptable salt thereof, or a solvate thereof.

### Item 13

The process of Item 12, which further comprises Step L shown below,

### Step L:

a compound of formula (12):

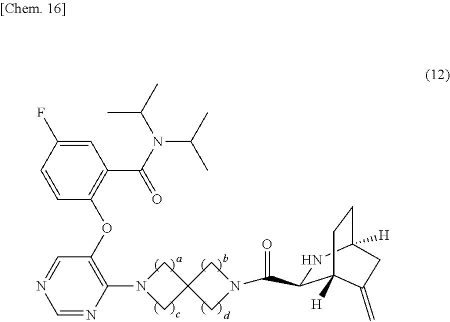

wherein a, b, c, and d are as defined above,
or a pharmaceutically acceptable salt thereof, or a solvate thereof is reacted with L(+)-tartaric acid in the presence of a solvent to prepare a compound of formula (12a):

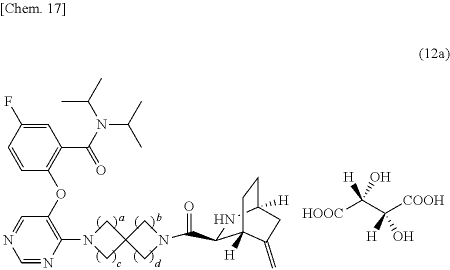

wherein a, b, c, and d are as defined above,
or a solvate thereof.

### Item 14

The process of any one of Items 1 to 13, which further comprises Step A shown below,

### Step A:

a compound of formula (1):

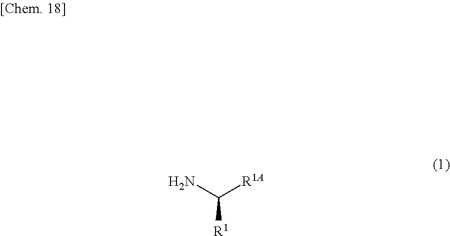

wherein R¹ is optionally-substituted C₁₋₆ alkyl, and R^{1A} is optionally-substituted C₆₋₁₀ aryl,
or a pharmaceutically acceptable salt thereof, or a solvate thereof is reacted with a compound of formula (1a):

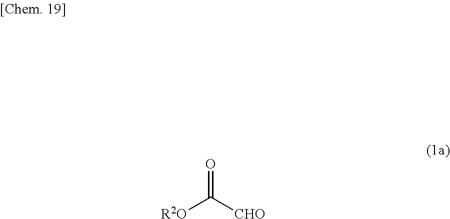

wherein R² is optionally-substituted C₁₋₆ alkyl,
or a pharmaceutically acceptable salt thereof, or a solvate thereof in the presence of a solvent to prepare a compound of formula (2):

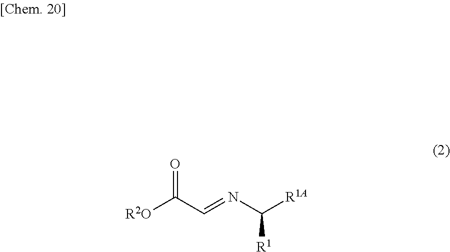

wherein R¹, R^{1A}, and R² are as defined above,
or a pharmaceutically acceptable salt thereof, or a solvate thereof.

### Item 15

The process of Item 14, which further comprises Step B shown below,

### Step B:

a compound of formula (2):

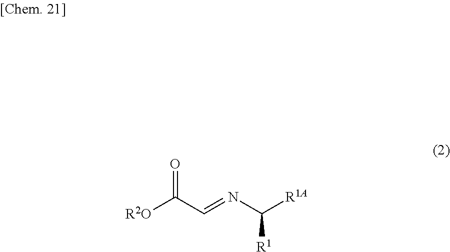

wherein R¹, R^{1A}, and R² are as defined above,
or a pharmaceutically acceptable salt thereof, or a solvate thereof is reacted with 1,3-cyclohexadiene in the presence of a solvent and an acid to prepare a compound of formula (3):

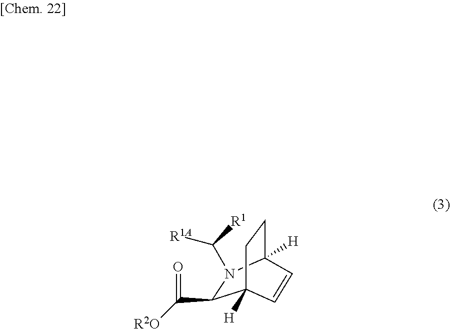

wherein R¹, R^{1A}, and R² are as defined above,
or a pharmaceutically acceptable salt thereof, or a solvate thereof.

### Item 16

The process of Item 15, which further comprises Step C shown below,

### Step C:

a compound of formula (3):

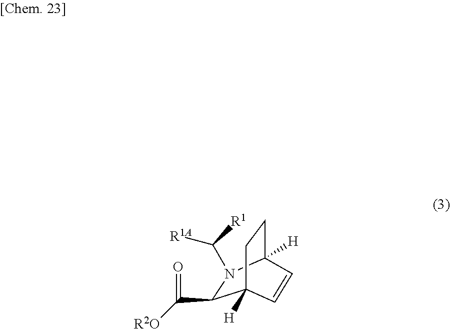

wherein R¹, R^{1A}, and R² are as defined above,
or a pharmaceutically acceptable salt thereof, or a solvate thereof is reacted with borane in the presence of a solvent, and then with a peroxide and a base to prepare a compound of formula (4):

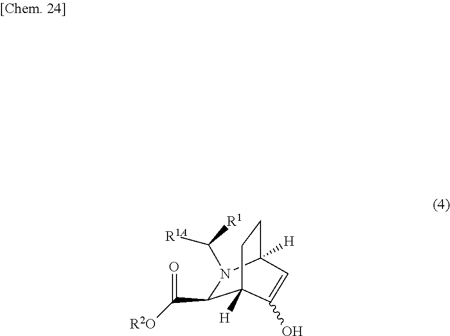

wherein R¹, R^{1A}, and R² are as defined above,
or a pharmaceutically acceptable salt thereof, or a solvate thereof.

### Item 17

The process of Item 16, which further comprises Step D shown below,

### Step D:

a compound of formula (4):

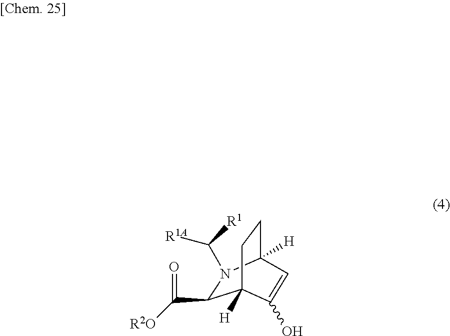

wherein R¹, R^{1A}, and R² are as defined above,
or a pharmaceutically acceptable salt thereof, or a solvate thereof is reacted with hydrogen in the presence of a catalyst for deprotecting the protecting group on the amino group to prepare a compound of formula (5):

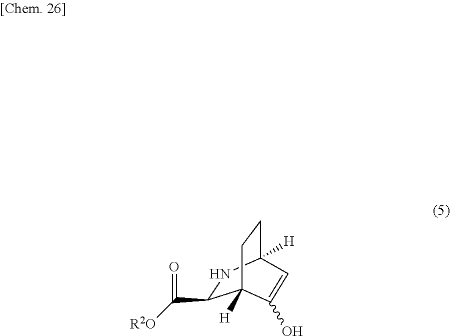

wherein R² is as defined above,
or a pharmaceutically acceptable salt thereof, or a solvate thereof.

### Item 18

The process of Item 17, which further comprises Step E shown below,

### Step E:

a compound of formula (5):

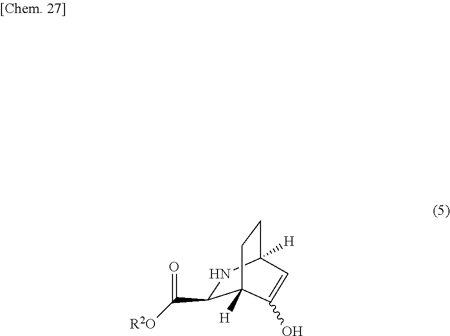

wherein R² is as defined above,
or a pharmaceutically acceptable salt thereof, or a solvate thereof is hydrolyzed of its ester group to prepare a compound of formula (6):

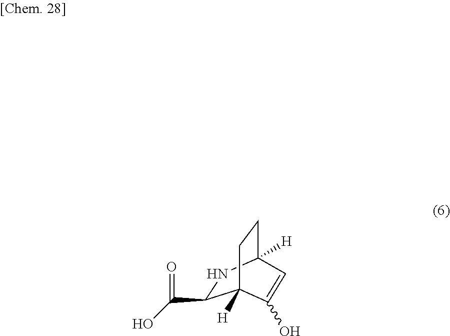 [SMILES: O=C(O)[C@H]1N[C@@H]2C=C(O)[C@H]1CC2]

or a pharmaceutically acceptable salt thereof, or a solvate thereof.

### Item 19

The process of Item 18, which further comprises Step F shown below,

### Step F:

a compound of formula (6):

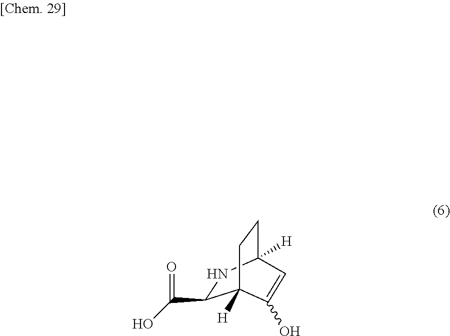 [SMILES: O=C(O)[C@H]1N[C@@H]2C=C(O)[C@H]1CC2]

or a pharmaceutically acceptable salt thereof, or a solvate thereof is protected of its amino group to prepare a compound of formula (7):

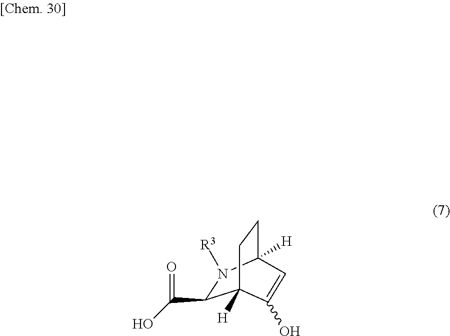

wherein R³ is as defined above,
or a pharmaceutically acceptable salt thereof, or a solvate thereof.

### Item 20

A process for preparing a compound of formula (12):

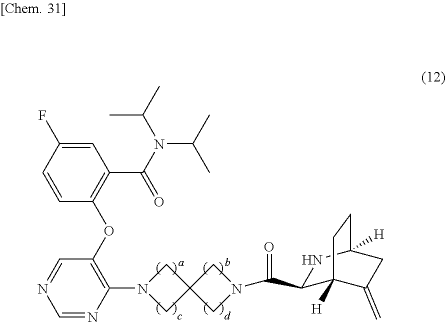

wherein a, b, c, and d are independently 1 or 2,
or a pharmaceutically acceptable salt thereof, or a solvate thereof, comprising Step M shown below,

### Step M:

a compound of formula (2):

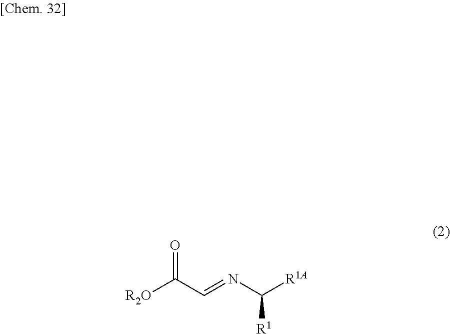

wherein R¹, R^{1A}, and R² are as defined above,
or a pharmaceutically acceptable salt thereof, or a solvate thereof is reacted with a compound of formula (2a):

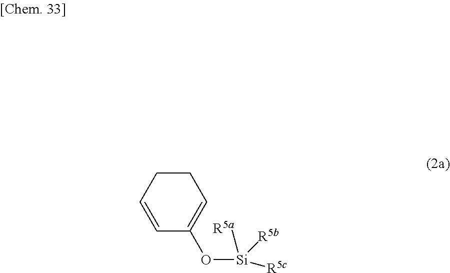

wherein R⁵, R^{5b}, and R^{5c} are independently C₁₋₆ alkyl or C₆₋₁₀ aryl,
or a pharmaceutically acceptable salt thereof, or a solvate thereof under an acidic condition in the presence of a solvent to prepare a compound of formula (13):

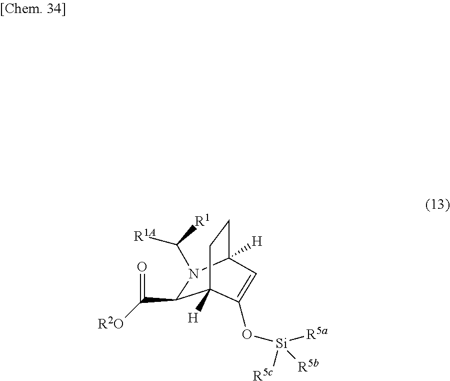

wherein R¹, R^{1A}, R², R^{5a}, R^{5b}, and R^{5c} are as defined above, or a pharmaceutically acceptable salt thereof, or a solvate thereof.

### Item 21

The process of Item 20, wherein the solvent in Step M comprises a halogen solvent.

### Item 22

The process of Item 20, wherein the solvent in Step M comprises dichloromethane and/or chloroform.

### Item 23

The process of Item 20, wherein the solvent in Step M comprises dichloromethane.

### Item 24

The process of any one of Items 20 to 23, wherein the reaction temperature in Step M is −100 °C to −30 °C.

### Item 25

The process of any one of Items 20 to 23, wherein the reaction temperature in Step M is −80 °C to −10 °C.

### Item 26

The process of any one of Items 20 to 25, wherein the acid used in Step M is methanesulfonic acid, trichloroacetic acid, dichloroacetic acid, difluoroacetic acid, trifluoroacetic acid, boron trifluoride-ethyl ether complex, or a mixture of trifluoroacetic acid and boron trifluoride-ethyl ether complex.

### Item 27

The process of any one of Items 20 to 25, wherein the acid used in Step M is a mixture of trifluoroacetic acid and boron trifluoride-ethyl ether complex.

### Item 28

The process of any one of Items 20 to 27, which further comprises Step A shown below,

### Step A:

a compound of formula (1):

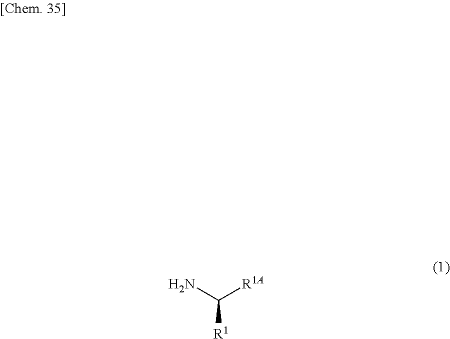

wherein R¹ and R^{1A} are as defined above,
or a pharmaceutically acceptable salt thereof, or a solvate thereof is reacted with a compound of formula (1a):

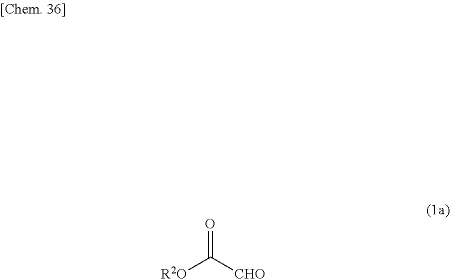

wherein R² is as defined above,
or a pharmaceutically acceptable salt thereof, or a solvate thereof in the presence of a solvent to prepare a compound of formula (2):

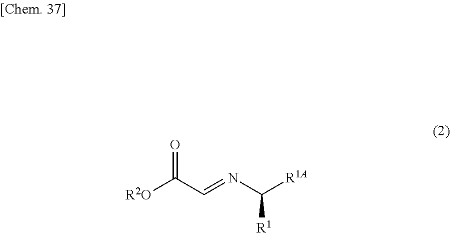

wherein R¹, R^{1A}, and R² are as defined above,
or a pharmaceutically acceptable salt thereof, or a solvate thereof.

### Item 29

The process of any one of Items 20 to 28, which further comprises Step N shown below,

### Step N:

a compound of formula (13):

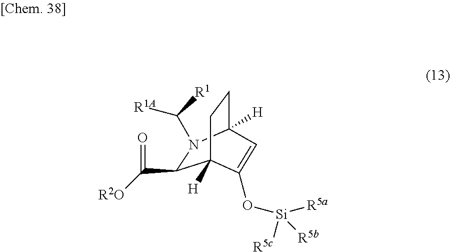

wherein R¹, R^{1A}, R², R^{5a}, R^{5b}, and R^{5c} are as defined above,
or a pharmaceutically acceptable salt thereof, or a solvate thereof is hydrolyzed of its silyl enol ether group to prepare a compound of formula (14):

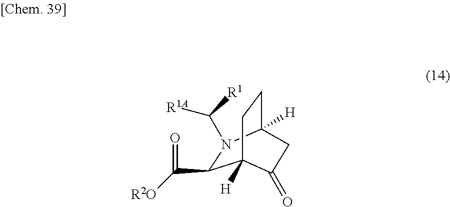

wherein R¹, R^{1A}, and R² are as defined above,
or a pharmaceutically acceptable salt thereof, or a solvate thereof.

### Item 30

The process of Item 29, which further comprises Step O shown below,

### Step O:

a compound of formula (14):

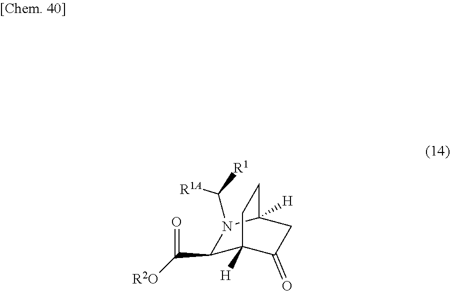

wherein R¹, R^{1A}, and R² are as defined above,
or a pharmaceutically acceptable salt thereof, or a solvate thereof is reacted with hydrogen in the presence of a catalyst for deprotecting the protecting group on the amino group to prepare a compound of formula (15):

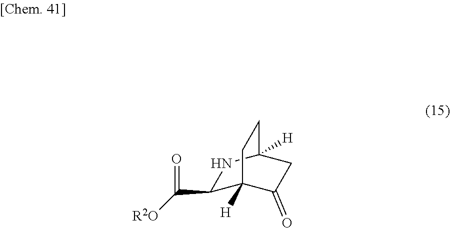

wherein R² is as defined above,
or a pharmaceutically acceptable salt thereof, or a solvate thereof.

### Item 31

The process of Item 30, which further comprises Step P shown below,

### Step P:

a compound of formula (15):

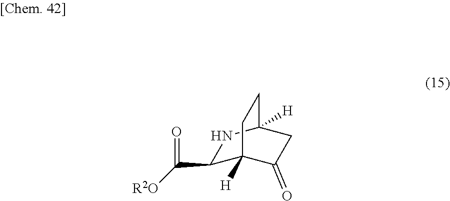

wherein R² is as defined above,
or a pharmaceutically acceptable salt thereof, or a solvate thereof is protected of its amino group to prepare a compound of formula (16):

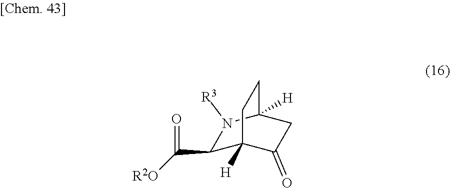

wherein R² and R³ are as defined above,
or a pharmaceutically acceptable salt thereof, or a solvate thereof.

### Item 32

The process of Item 31, which further comprises Step Q shown below,

### Step Q:

a compound of formula (16):

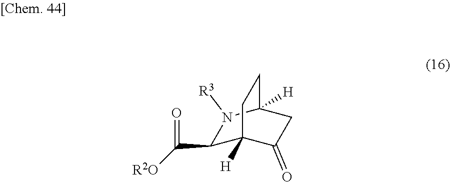

wherein R² and R³ are as defined above,
or a pharmaceutically acceptable salt thereof, or a solvate thereof is hydrolyzed of its ester group to prepare a compound of formula (8):

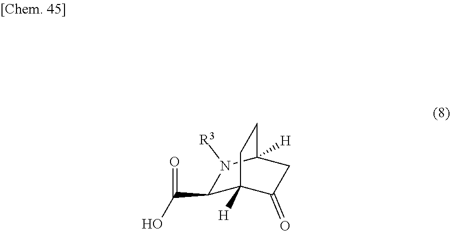

wherein R³ is as defined above,
or a pharmaceutically acceptable salt thereof, or a solvate thereof.

### Item 33

The process of Item 32, which further comprises Step H shown below,

### Step H:

a compound of formula (8):

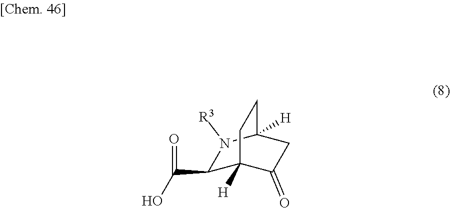

wherein R³ is as defined above,
or a pharmaceutically acceptable salt thereof, or a solvate thereof is reacted with a compound of formula (8a):

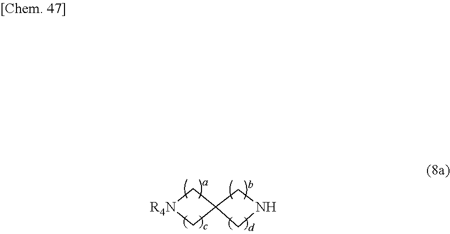

wherein R⁴, a, b, c, and d are as defined above,
or a pharmaceutically acceptable salt thereof, or a solvate thereof in the presence of a solvent and a condensation agent to prepare a compound of formula (9):

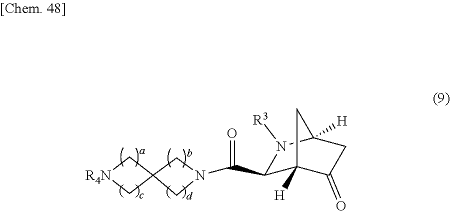

wherein R³, R⁴, a, b, c, and d are as defined above,
or a pharmaceutically acceptable salt thereof, or a solvate thereof.

### Item 34

The process of Item 33, which further comprises Step I shown below,

### Step I:

a compound of formula (9)

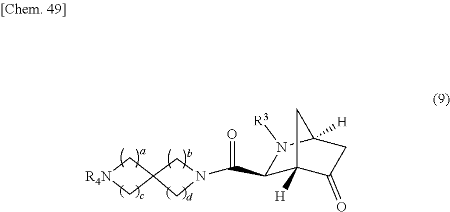

wherein R³, R⁴, a, b, c, and d are as defined above,
or a pharmaceutically acceptable salt thereof, or a solvate thereof is reacted with halogenated methyltriphenylphosphonium and a base in the presence of a solvent to prepare a compound of formula (10):

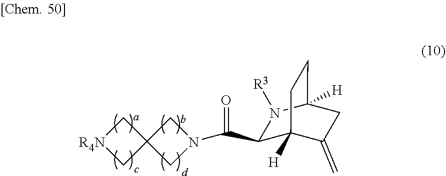

wherein R³, R⁴, a, b, c, and d are as defined above,
or a pharmaceutically acceptable salt thereof, or a solvate thereof.

### Item 35

The process of Item 34, which further comprises Step J shown below,

### Step J:

a compound of formula (10):

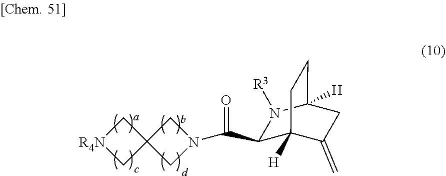

wherein R³, R⁴, a, b, c, and d are as defined above,
or a pharmaceutically acceptable salt thereof, or a solvate thereof is reacted with an acid to prepare a compound of formula (11):

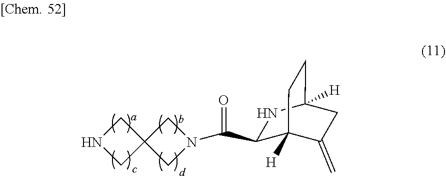

wherein a, b, c, and d are as defined above,
or a pharmaceutically acceptable salt thereof, or a solvate thereof.

### Item 36

The process of Item 35, which further comprises Step K shown below,

### Step K:

a compound of formula (11):

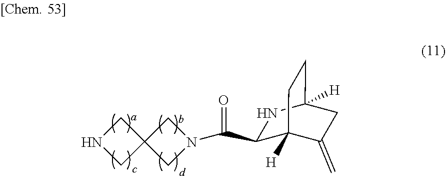

wherein a, b, c, and d are as defined above,
or a pharmaceutically acceptable salt thereof, or a solvate thereof is reacted with a compound of formula (11a):

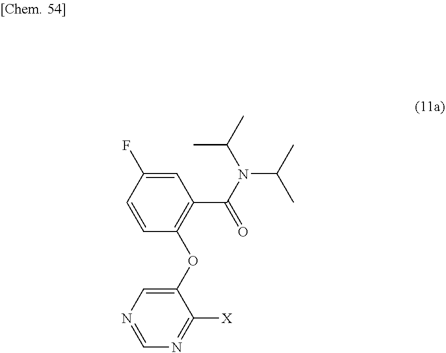

wherein X is fluorine, chlorine, bromine, iodine, p-toluenesulfonyl group, or methanesulfonyl group,
or a pharmaceutically acceptable salt thereof, or a solvate thereof in the presence of a solvent and a base to prepare a compound of formula (12):

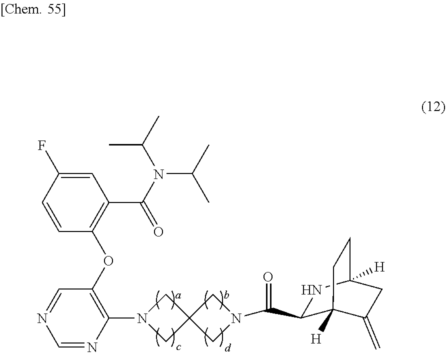

wherein a, b, c, and d are as defined above,
or a pharmaceutically acceptable salt thereof, or a solvate thereof.

### Item 37

The process of Item 36, which further comprises Step L shown below,

### Step L:

a compound of formula (11):

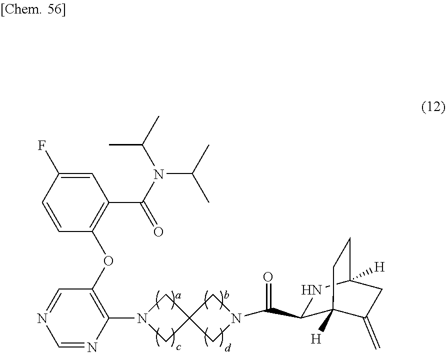

wherein a, b, c, and d are as defined above,
or a pharmaceutically acceptable salt thereof, or a solvate thereof is reacted with L(+)-tartaric acid in the presence of a solvent to prepare a compound of formula (12a):

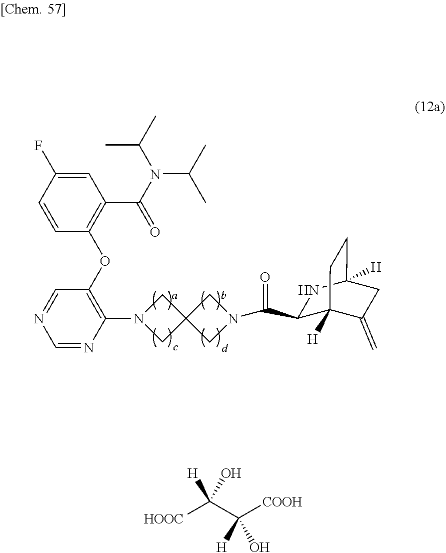

wherein a, b, c, and d are as defined above, or a solvate thereof.

### Item 38

A process for preparing a compound of formula (8):

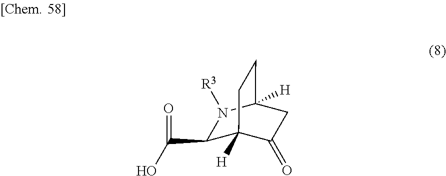

wherein R³ is a protecting group for an amino group,
or a pharmaceutically acceptable salt thereof, or a solvate thereof, comprising Step G shown below,

### Step G:

a compound of formula (7):

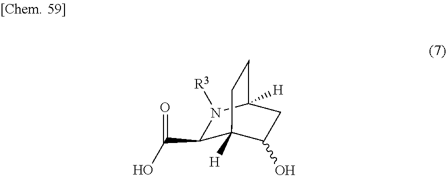

wherein R³ is as defined above,
or a pharmaceutically acceptable salt thereof, or a solvate thereof is reacted with an oxidation agent in the presence of a solvent to prepare a compound of formula (8):

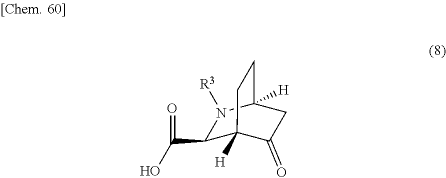

wherein R³ is as defined above,
or a pharmaceutically acceptable salt thereof, or a solvate thereof.

### Item 39

The process of Item 38, which further comprises Step A shown below,

### Step A:

a compound of formula (1):

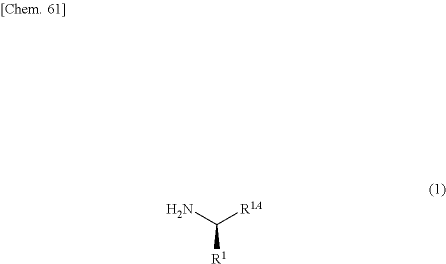

wherein R¹ is optionally-substituted C₁₋₆ alkyl, and R^{1A} is optionally-substituted C₆₋₁₀ aryl,
or a pharmaceutically acceptable salt thereof, or a solvate thereof is reacted with a compound of formula (1a):

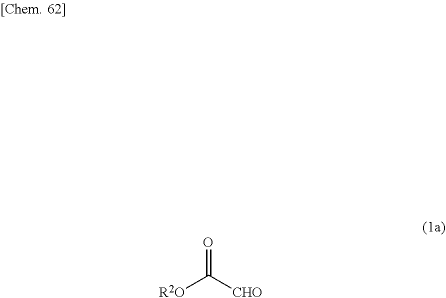

wherein R² is optionally-substituted C₁₋₆ alkyl,
or a pharmaceutically acceptable salt thereof, or a solvate thereof in the presence of a solvent to prepare a compound of formula (2):

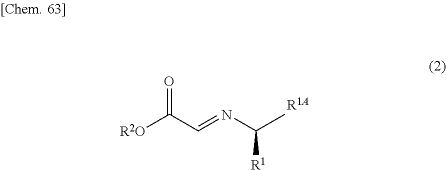

wherein R¹, R^{1A}, and R² are as defined above,
or a pharmaceutically acceptable salt thereof, or a solvate thereof.

### Item 40

The process of Item 39, which further comprises Step B shown below,

### Step B:

a compound of formula (2):

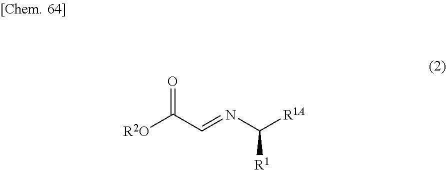

wherein R¹, R^{1A}, and R² are as defined above,
or a pharmaceutically acceptable salt thereof, or a solvate thereof is reacted with 1,3-cyclohexadiene in the presence of a solvent and an acid to prepare a compound of formula (3):

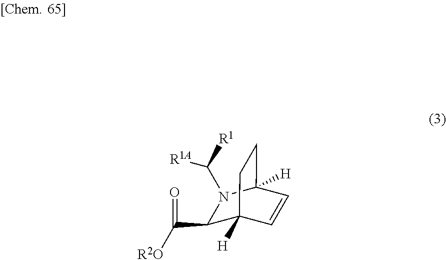

wherein R¹, R^{1A}, and R² are as defined above,
or a pharmaceutically acceptable salt thereof, or a solvate thereof.

### Item 41

The process of Item 40, which further comprises Step C shown below,

### Step C:

a compound of formula (3):

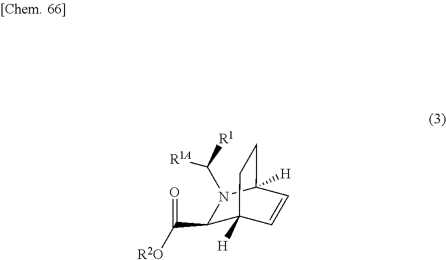

wherein R¹, R^{1A}, and R² are as defined above,
or a pharmaceutically acceptable salt thereof, or a solvate thereof is reacted with borane in the presence of a solvent, and then with a peroxide and a base to prepare a compound of formula (4):

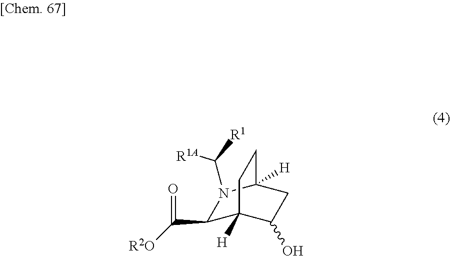

wherein R¹, R^{1A}, and R² are as defined above,
or a pharmaceutically acceptable salt thereof, or a solvate thereof.

### Item 42

The process of Item 41, which further comprises Step D shown below,

### Step D:

a compound of formula (4):

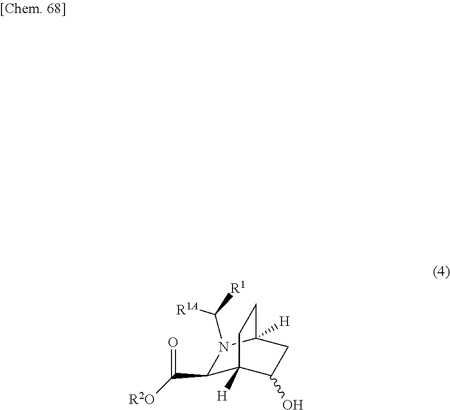

wherein R¹, R^{1A}, and R² are as defined above,
or a pharmaceutically acceptable salt thereof, or a solvate thereof is reacted with hydrogen in the presence of a catalyst for deprotecting the protecting group on the amino group to prepare a compound of formula (5):

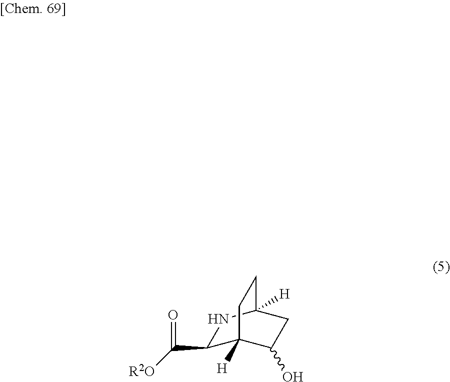

wherein R² is as defined above,
or a pharmaceutically acceptable salt thereof, or a solvate thereof.

### Item 43

The process of Item 42, which further comprises Step E shown below,

### Step E:

a compound of formula (5):

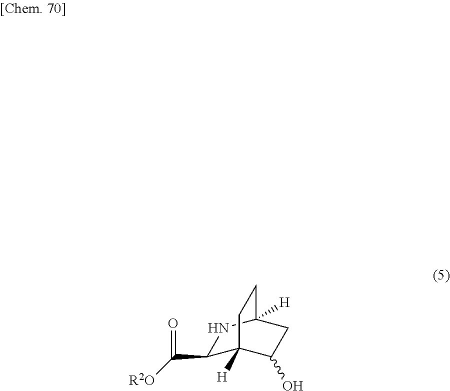

wherein R² is as defined above,
or a pharmaceutically acceptable salt thereof, or a solvate thereof is hydrolyzed of its ester group to prepare a compound of formula (6):

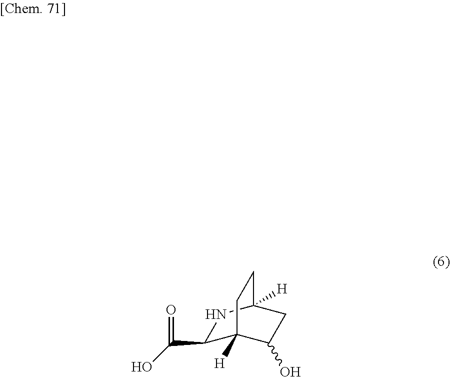 [SMILES: O=C(O)[C@H]1N[C@H]2CC[C@@H]1C(O)C2]

or a pharmaceutically acceptable salt thereof, or a solvate thereof.

### Item 44

The process of Item 43, which further comprises Step F shown below,

### Step F:

a compound of formula (6):

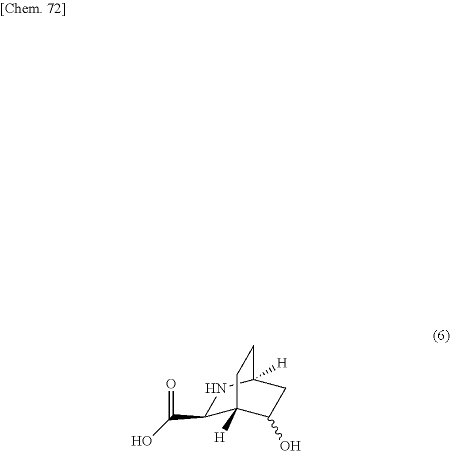 [SMILES: O=C(O)[C@H]1N[C@H]2CC[C@@H]1C(O)C2]

or a pharmaceutically acceptable salt thereof, or a solvate thereof is protected of its amino group to prepare a compound of formula (7):

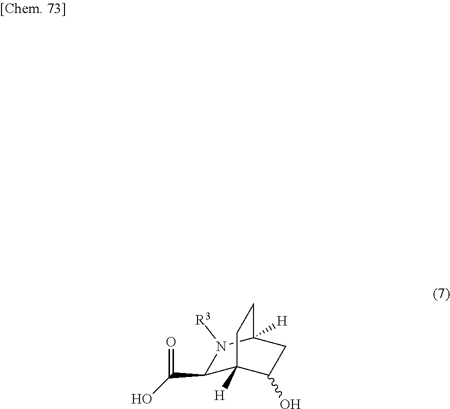

wherein R³ is as defined above,
or a pharmaceutically acceptable salt thereof, or a solvate thereof.

### Item 45

A process for preparing a compound of formula (8):

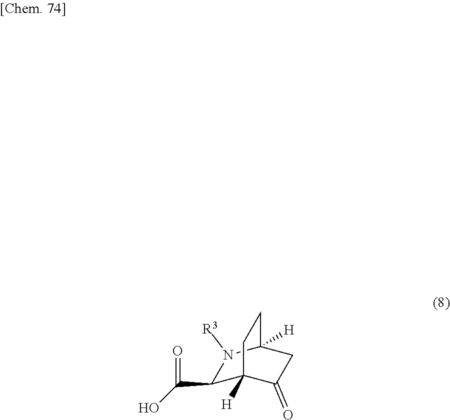

wherein R³ is a protecting group for an amino group,
or a pharmaceutically acceptable salt thereof, or a solvate thereof, comprising Step M shown below,

### Step M:

a compound of formula (2):

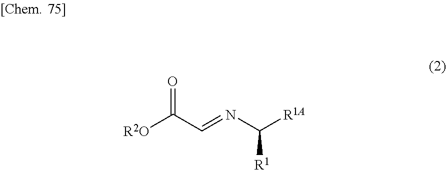

wherein R¹, R^{1A}, and R² are as defined above,
or a pharmaceutically acceptable salt thereof, or a solvate thereof is reacted with a compound of formula (2a):

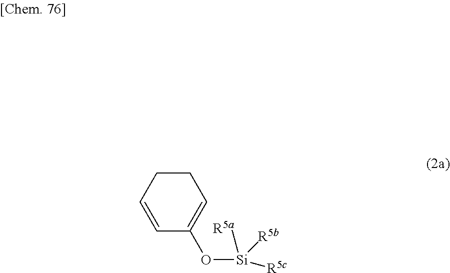

wherein R^{5a}, R^{5b}, and R^{5c} are independently C₁₋₆ alkyl or C₆₋₁₀ aryl,
or a pharmaceutically acceptable salt thereof, or a solvate thereof under an acidic condition in the presence of a solvent to prepare a compound of formula (13):

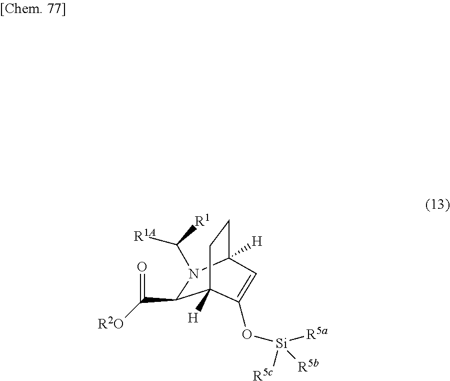

wherein R¹, R^{1A}, R², R^{5a}, R^{5b}, and R^{5c} are as defined above,
or a pharmaceutically acceptable salt thereof, or a solvate thereof.

### Item 46

The process of Item 45, which further comprises Step A shown below,

### Step A:

a compound of formula (1):

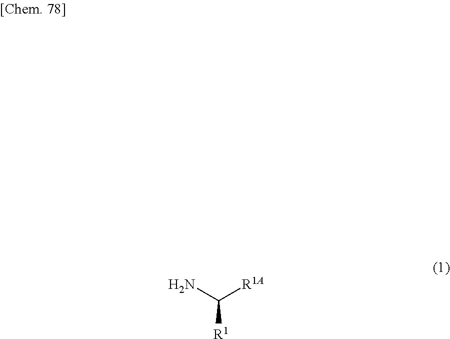

wherein R¹ is optionally-substituted C₁₋₆ alkyl, and R^{1A} is optionally-substituted C₆₋₁₀ aryl,
or a pharmaceutically acceptable salt thereof, or a solvate thereof is reacted with a compound of formula (1a):

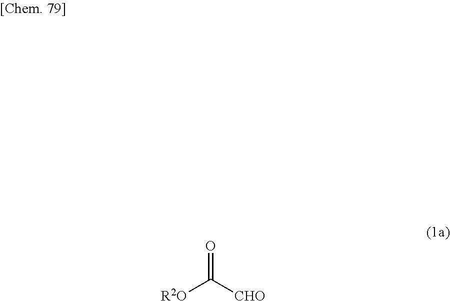

wherein R² is optionally-substituted C₁₋₆ alkyl,
or a pharmaceutically acceptable salt thereof, or a solvate thereof in the presence of a solvent to prepare a compound of formula (2):

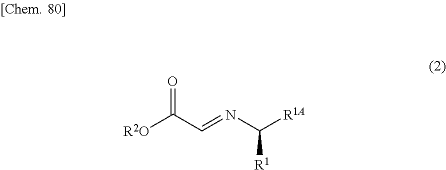

wherein R¹, R^{1A}, and R² are as defined above,
or a pharmaceutically acceptable salt thereof, or a solvate thereof.

### Item 47

The process of Item 45 or 46, which further comprises Step N shown below,

### Step N:

a compound of formula (13)

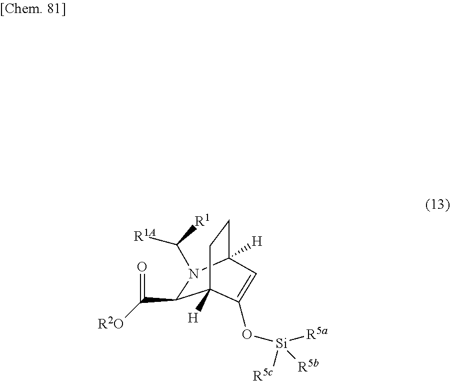

wherein R¹, R^{1A}, R², R^{5a}, R^{5b}, and R^{5c} are as defined above,
or a pharmaceutically acceptable salt thereof, or a solvate thereof is hydrolyzed of its silyl enol ether group to prepare a compound of formula (14):

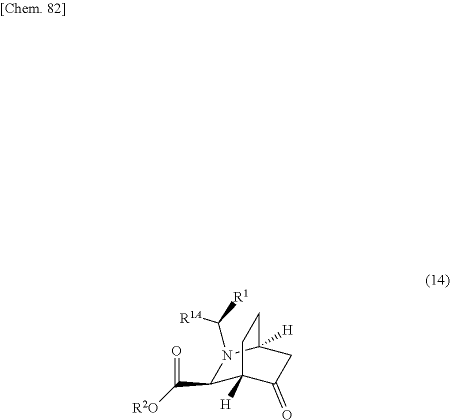

wherein R¹, R^{1A}, and R² are as defined above,
or a pharmaceutically acceptable salt thereof, or a solvate thereof.

### Item 48

The process of Item 47, which further comprises Step O shown below,

### Step O:

a compound of formula (14):

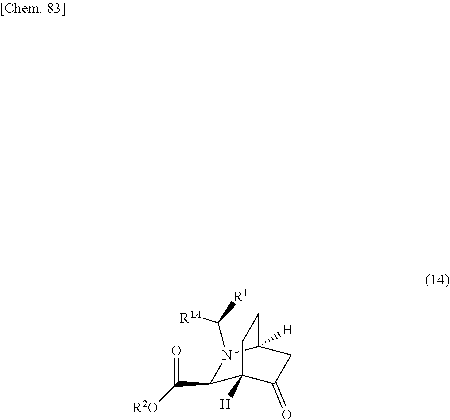

wherein R¹, R^{1A}, and R² are as defined above,
or a pharmaceutically acceptable salt thereof, or a solvate thereof is reacted with hydrogen in the presence of a catalyst for deprotecting the protecting group on the amino group to prepare a compound of formula (15):

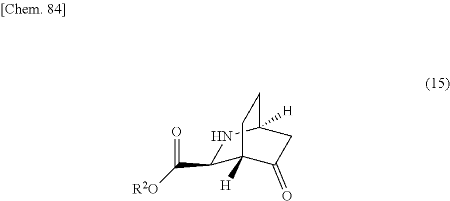

wherein R² is as defined above,
or a pharmaceutically acceptable salt thereof, or a solvate thereof.

### Item 49

The process of Item 48, which further comprises Step P shown below,

### Step P:

a compound of formula (15):

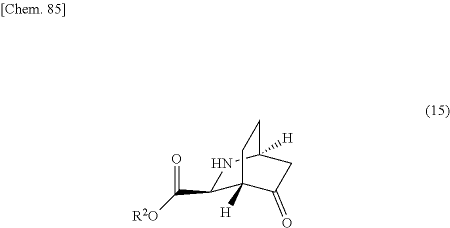

wherein R² is as defined above,
or a pharmaceutically acceptable salt thereof, or a solvate thereof is protected of its amino group to prepare a compound of formula (16):

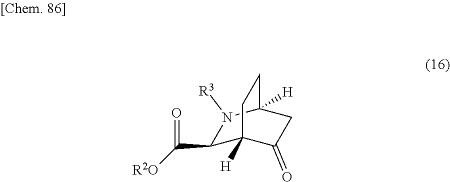

wherein R² and R³ are as defined above,
or a pharmaceutically acceptable salt thereof, or a solvate thereof.

### Item 50

The process of Item 49, which further comprises Step Q shown below,

### Step Q:

a compound of formula (16):

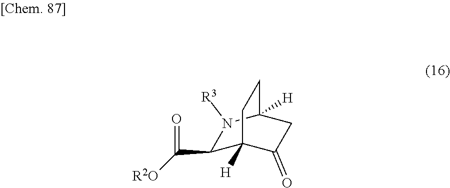

wherein R² and R³ are as defined above,
or a pharmaceutically acceptable salt thereof, or a solvate thereof is hydrolyzed of its ester group to prepare a compound of formula (8):

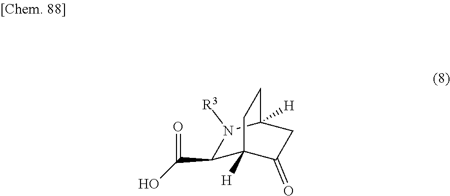

wherein R³ is as defined above,
or a pharmaceutically acceptable salt thereof, or a solvate thereof.

### Item 51

A process for preparing a compound of formula (12):

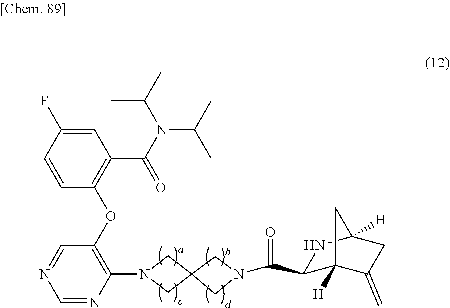

wherein a, b, c, and d are independently 1 or 2,
or a pharmaceutically acceptable salt thereof, or a solvate thereof, comprising Step H shown below,

### Step H:

a compound of formula (8):

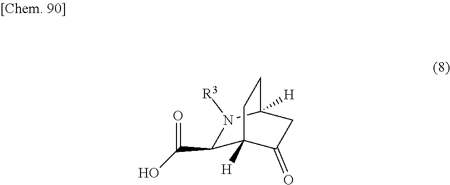

wherein R³ is as defined above,
or a pharmaceutically acceptable salt thereof, or a solvate thereof is reacted with a compound of formula (8a):

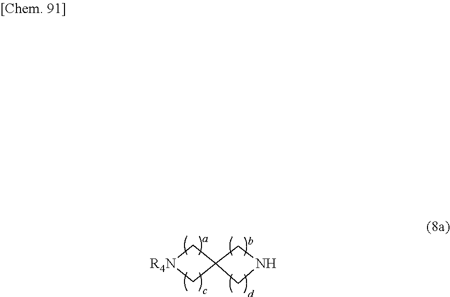

wherein R⁴, a, b, c, and d are as defined above,
or a pharmaceutically acceptable salt thereof, or a solvate thereof in the presence of a solvent and a condensation agent to prepare a compound of formula (9):

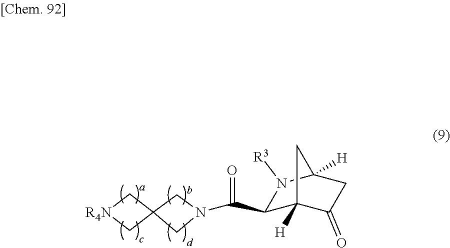

wherein R³, R⁴, a, b, c, and d are as defined above,
or a pharmaceutically acceptable salt thereof, or a solvate thereof.

### Item 52

The process of Item 51, which further comprises Step I shown below,

### Step I:

a compound of formula (9)

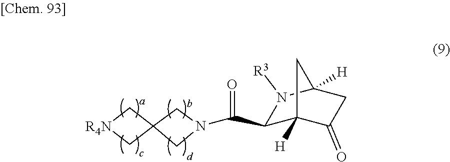

wherein R³, R⁴, a, b, c, and d are as defined above,
or a pharmaceutically acceptable salt thereof, or a solvate thereof is reacted with halogenated methyltriphenylphosphonium, trialkylsilylmethyl anion, or methylsulfone derivative, in the presence of a solvent and a base to prepare a compound of formula (10):

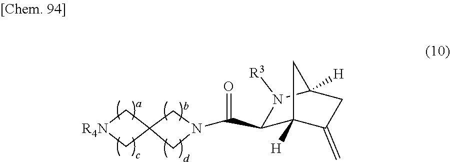

wherein R³, R⁴, a, b, c, and d are as defined above,
or a pharmaceutically acceptable salt thereof, or a solvate thereof.

### Item 53

The process of Item 52, which further comprises Step J shown below,

### Step J:

a compound of formula (10):

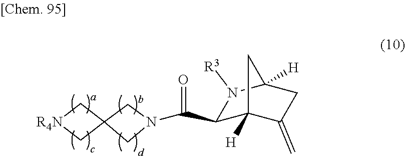

wherein R³, R⁴, a, b, c, and d are as defined above,
or a pharmaceutically acceptable salt thereof, or a solvate thereof is reacted with an acid to prepare a compound of formula (11):

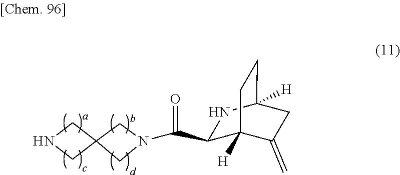

wherein a, b, c, and d are as defined above,
or a pharmaceutically acceptable salt thereof, or a solvate thereof.

### Item 54

The process of Item 53, which further comprises Step K shown below,

### Step K:

a compound of formula (11):

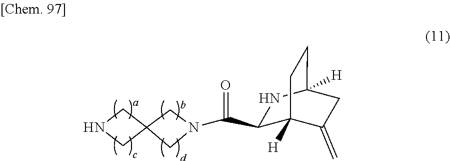

wherein a, b, c, and d are as defined above,
or a pharmaceutically acceptable salt thereof, or a solvate thereof is reacted with a compound of formula (11a):

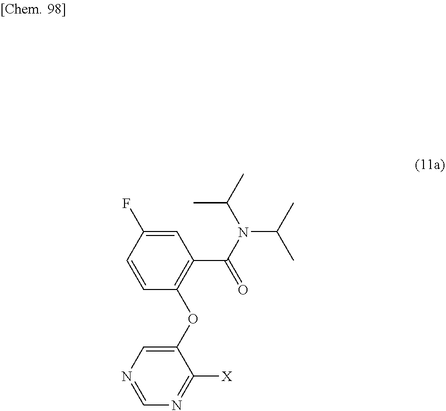

wherein X is fluorine, chlorine, bromine, iodine, p-toluenesulfonyl group, or methanesulfonyl group,
or a pharmaceutically acceptable salt thereof, or a solvate thereof in the presence of a solvent and a base to prepare a compound of formula (12):

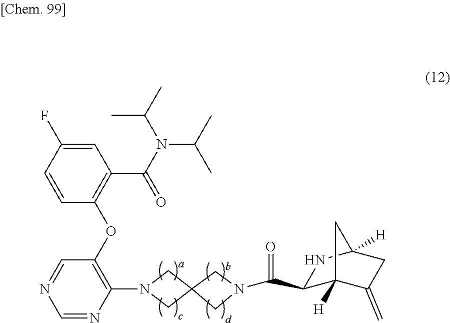

wherein a, b, c, and d are as defined above,
or a pharmaceutically acceptable salt thereof, or a solvate thereof.

### Item 55

The process of Item 54, which further comprises Step L shown below,

### Step L:

a compound of formula (12):

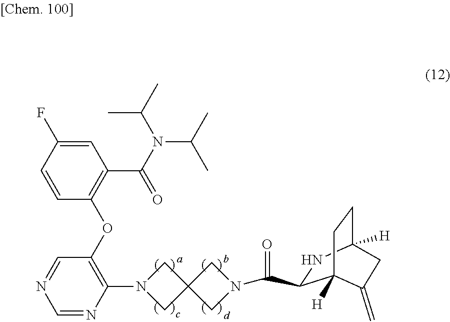

wherein a, b, c, and d are as defined above,
or a pharmaceutically acceptable salt thereof, or a solvate thereof is reacted with L(+)-tartaric acid in the presence of a solvent to prepare a compound of formula (12a):

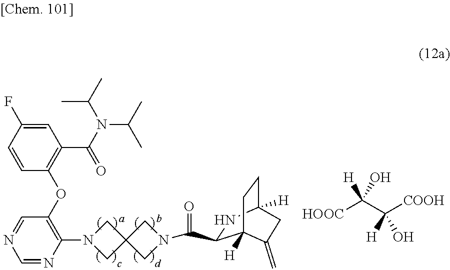

wherein a, b, c, and d are as defined above,
or a solvate thereof.

### Item 56

A mixture comprising 5-fluoro-2-[(4-{7-[(1S,3S,4R)-5-methylidene-2-azabicyclo[2.2.2]octane-3-carbonyl]-2,7-diazaspiro[3.5]nonan-2-yl}pyrimidin-5-yl)oxy]-N,N-di(propan-2-yl)benzamide or a pharmaceutically acceptable salt thereof, or a solvate thereof, and Compound A or a pharmaceutically acceptable salt thereof, or a solvate thereof.

### Item 57

The mixture of Item 56, wherein Compound A or a pharmaceutically acceptable salt thereof, or a solvate thereof is present in amount of about 0.15% or less of the mixture as determined by high performance liquid chromatography (HPLC).

### Item 58

The mixture of Item 56, wherein Compound A or a pharmaceutically acceptable salt thereof, or a solvate thereof is present in amount of about 0.1% or less of the mixture as determined by high performance liquid chromatography (HPLC).

### Item 59

The mixture of Item 56, wherein Compound A or a pharmaceutically acceptable salt thereof, or a solvate thereof is present in amount of about 0.05% or less of the mixture as determined by high performance liquid chromatography (HPLC).

### Item 60

The mixture according to any one of Items 56 to 59, further comprising Compound B or a pharmaceutically acceptable salt thereof, or a solvate thereof.

### Item 61

The mixture according to Item 60, wherein Compound B or a pharmaceutically acceptable salt thereof, or a solvate thereof is present in amount of about 0.15% or less of the mixture as determined by high performance liquid chromatography (HPLC).

### Item 62

The mixture according to Item 60, wherein Compound B or a pharmaceutically acceptable salt thereof, or a solvate thereof is present in amount of about 0.1% or less of the mixture as determined by high performance liquid chromatography (HPLC).

### Item 63

The mixture according to Item 60, wherein Compound B or a pharmaceutically acceptable salt thereof, or a solvate thereof is present in amount of about 0.05% or less of the mixture as determined by high performance liquid chromatography (HPLC).

### Item 64

The mixture according to Item 60, further comprising Compound C or a pharmaceutically acceptable salt thereof, or a solvate thereof.

### Item 65

The mixture according to Item 64, wherein Compound C or a pharmaceutically acceptable salt thereof, or a solvate thereof is present in amount of about 0.15% or less of the mixture as determined by high performance liquid chromatography (HPLC).

### Item 66

The mixture according to Item 64, wherein Compound C or a pharmaceutically acceptable salt thereof, or a solvate thereof is present in amount of about 0.1% or less of the mixture as determined by high performance liquid chromatography (HPLC).

### Item 67

The mixture according to Item 64, wherein Compound C or a pharmaceutically acceptable salt thereof, or a solvate thereof is present in amount of about 0.06% or less of the mixture as determined by high performance liquid chromatography (HPLC).

### Item 68

A mixture comprising 5-fluoro-2-[(4-{7-[(1S,3S,4R)-5-methylidene-2-azabicyclo[2.2.2]octane-3-carbonyl]-2,7-diazaspiro[3.5]nonan-2-yl}pyrimidin-5-yl)oxy]-N,N-di(propan-2-yl)benzamide or a pharmaceutically acceptable salt thereof, or a solvate thereof, and Compound B or a pharmaceutically acceptable salt thereof, or a solvate thereof.

### Item 69

The mixture of Item 68, wherein Compound B or a pharmaceutically acceptable salt thereof, or a solvate thereof is present in amount of about 0.15% or less of the mixture as determined by high performance liquid chromatography (HPLC).

### Item 70

The mixture of Item 68, wherein Compound B or a pharmaceutically acceptable salt thereof, or a solvate thereof is present in amount of about 0.1% or less of the mixture as determined by high performance liquid chromatography (HPLC).

### Item 71

The mixture of Item 68, wherein Compound B or a pharmaceutically acceptable salt thereof, or a solvate thereof is present in amount of about 0.05% or less of the mixture as determined by high performance liquid chromatography (HPLC).

### Item 72

The mixture according to any one of Items 68 to 71, further comprising Compound C or a pharmaceutically acceptable salt thereof, or a solvate thereof.

### Item 73

The mixture according to Item 72, wherein Compound C or a pharmaceutically acceptable salt thereof, or a solvate thereof is present in amount of about 0.15% or less of the mixture as determined by high performance liquid chromatography (HPLC).

### Item 74

The mixture according to Item 72, wherein Compound C or a pharmaceutically acceptable salt thereof, or a solvate thereof is present in amount of about 0.1% or less of the mixture as determined by high performance liquid chromatography (HPLC).

### Item 75

The mixture according to Item 72, wherein Compound C or a pharmaceutically acceptable salt thereof, or a solvate thereof is present in amount of about 0.06% or less of the mixture as determined by high performance liquid chromatography (HPLC).

### Item 76

A mixture comprising 5-fluoro-2-[(4-{7-[(1S,3S,4R)-5-methylidene-2-azabicyclo[2.2.2]octane-3-carbonyl]-2,7-diazaspiro[3.5]nonan-2-yl}pyrimidin-5-yl)oxy]-N,N-di(propan-2-yl)benzamide or a pharmaceutically acceptable salt thereof, or a solvate thereof, and Compound C or a pharmaceutically acceptable salt thereof, or a solvate thereof.

### Item 77

The mixture of Item 76, wherein Compound C or a pharmaceutically acceptable salt thereof, or a solvate thereof is present in amount of about 0.15% or less of the mixture as determined by high performance liquid chromatography (HPLC).

### Item 78

The mixture of Item 76, wherein Compound C or a pharmaceutically acceptable salt thereof, or a solvate thereof is present in amount of about 0.1% or less of the mixture as determined by high performance liquid chromatography (HPLC).

### Item 79

The mixture of Item 76, wherein Compound C or a pharmaceutically acceptable salt thereof, or a solvate thereof is present in amount of about 0.06% or less of the mixture as determined by high performance liquid chromatography (HPLC).

### Item A1

A compound of formula (12):

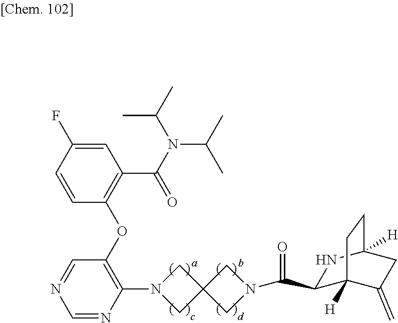

wherein a, b, c, and d are independently 1 or 2,
or a pharmaceutically acceptable salt thereof, or a solvate thereof, which is prepared by a process comprising Step G shown below,

### Step G:

a compound of formula (7):

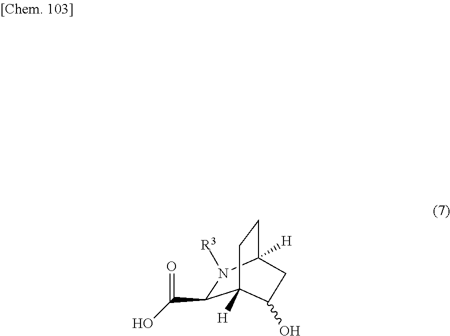

wherein R³ is a protecting group for an amino group,
or a pharmaceutically acceptable salt thereof, or a solvate thereof is reacted with an oxidation agent in the presence of a solvent to prepare a compound of formula (8):

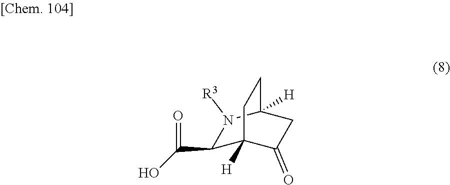

wherein R³ is as defined above,
or a pharmaceutically acceptable salt thereof, or a solvate thereof.

### Item A2

The compound of Item A1, or a pharmaceutically acceptable salt thereof, or a solvate thereof, wherein the process further comprises Step H shown below,

### Step H:

a compound of formula (8):

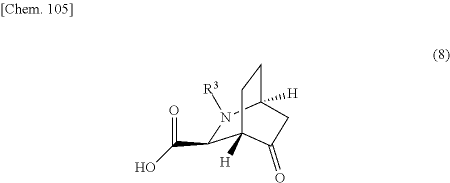

wherein R³ is as defined above,
or a pharmaceutically acceptable salt thereof, or a solvate thereof is reacted with a compound of formula (8a):

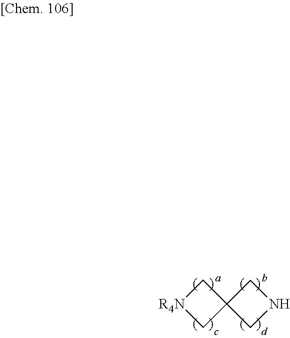

wherein R⁴, a, b, c, and d are as defined above,
or a pharmaceutically acceptable salt thereof, or a solvate thereof in the presence of a solvent and a condensation agent to prepare a compound of formula (9):

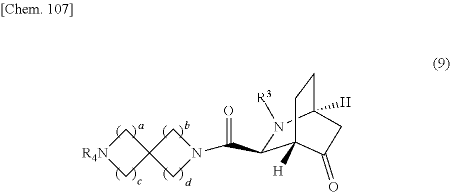

wherein R³, R⁴, a, b, c, and d are as defined above,
or a pharmaceutically acceptable salt thereof, or a solvate thereof.

### Item A3

The compound of Item A2, or a pharmaceutically acceptable salt thereof, or a solvate thereof, wherein the process further comprises Step I shown below,

### Step I:

a compound of formula (9):

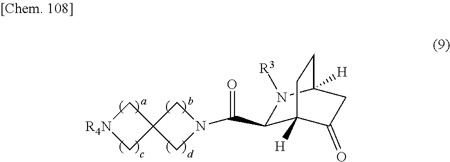

wherein R³, R⁴, a, b, c, and d are as defined above,
or a pharmaceutically acceptable salt thereof, or a solvate thereof is reacted with halogenated methyltriphenylphosphonium, trialkylsilylmethyl anion, or methylsulfone derivative, in the presence of a solvent and a base to prepare a compound of formula (10):

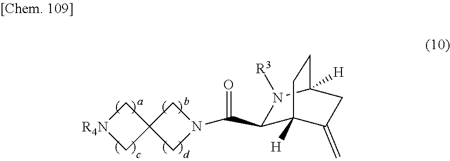

wherein R³, R⁴, a, b, c, and d are as defined above,
or a pharmaceutically acceptable salt thereof, or a solvate thereof.

### Item A4

The compound of Item A3, or a pharmaceutically acceptable salt thereof, or a solvate thereof, wherein the process further comprises Step J shown below,

### Step J:

a compound of formula (10):

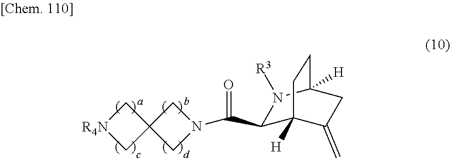

wherein R³, R⁴, a, b, c, and d are as defined above,
or a pharmaceutically acceptable salt thereof, or a solvate thereof is reacted with an acid to prepare a compound of formula (11):

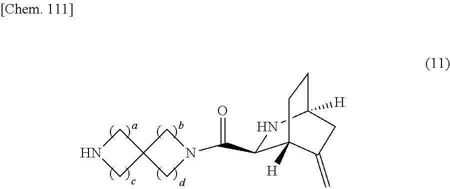

wherein a, b, c, and d are as defined above,
or a pharmaceutically acceptable salt thereof, or a solvate thereof.

### Item A5

The compound of Item A4, or a pharmaceutically acceptable salt thereof, or a solvate thereof, wherein the process further comprises Step K shown below,

### Step K:

a compound of formula (11):

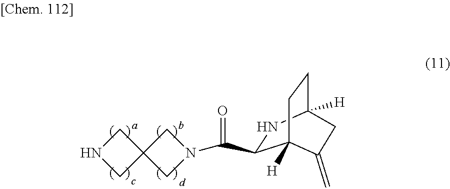

wherein a, b, c, and d are as defined above,
or a pharmaceutically acceptable salt thereof, or a solvate thereof is reacted with a compound of formula (11a):

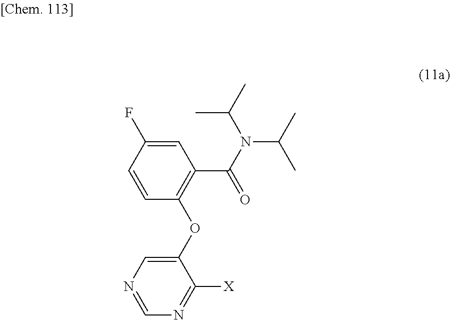

wherein X is fluorine, chlorine, bromine, iodine, p-toluenesulfonyl group, or methanesulfonyl group,
or a pharmaceutically acceptable salt thereof, or a solvate thereof in the presence of a solvent and a base to prepare a compound of formula (12):

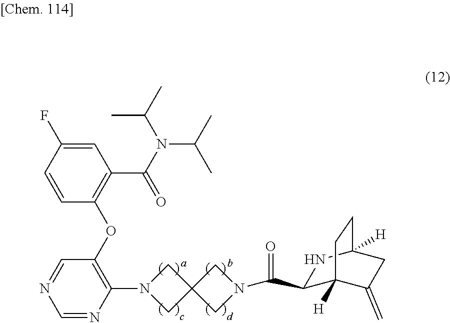

wherein a, b, c, and d are as defined above,
or a pharmaceutically acceptable salt thereof, or a solvate thereof.

### Item A6

The compound of Item A5, or a pharmaceutically acceptable salt thereof, or a solvate thereof, wherein the process further comprises Step L shown below,

### Step L:

a compound of formula (12):

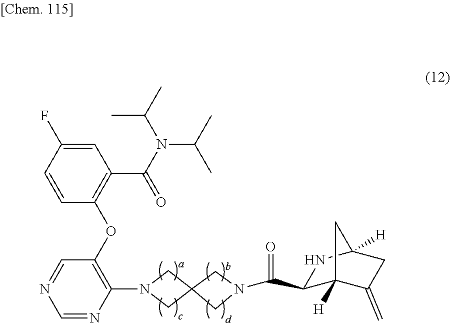

wherein a, b, c, and d are as defined above,
or a pharmaceutically acceptable salt thereof, or a solvate thereof is reacted with L(+)-tartaric acid in the presence of a solvent to prepare a compound of formula (12a):

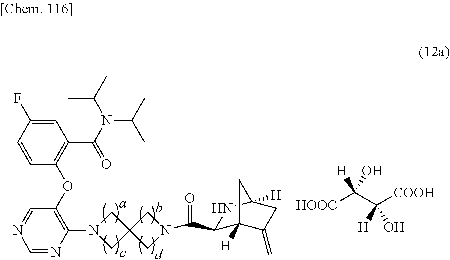

wherein a, b, c, and d are as defined above,
or a solvate thereof.

### Item A7

The compound of any one of Items A1 to A6, or a pharmaceutically acceptable salt thereof, or a solvate thereof, wherein the process further comprises Step A shown below,

### Step A:

a compound of formula (1):

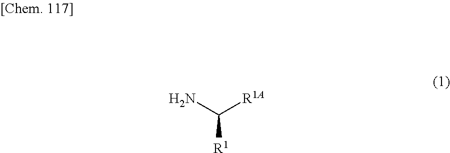

wherein R¹ is optionally-substituted C₁₋₆ alkyl, and R^{1A} is optionally-substituted C₆₋₁₀ aryl,
or a pharmaceutically acceptable salt thereof, or a solvate thereof is reacted with a compound of formula (1a):

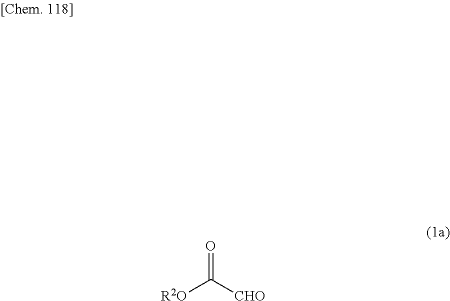

wherein R² is optionally-substituted C₁₋₆ alkyl,
or a pharmaceutically acceptable salt thereof, or a solvate thereof in the presence of a solvent to prepare a compound of formula (2):

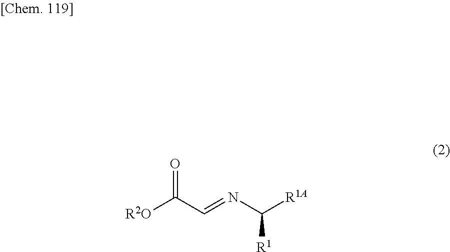

wherein R¹, R^{1A}, and R² are as defined above,
or a pharmaceutically acceptable salt thereof, or a solvate thereof.

### Item A8

The compound of Item A7, or a pharmaceutically acceptable salt thereof, or a solvate thereof, wherein the process further comprises Step B shown below,

### Step B:

a compound of formula (2):

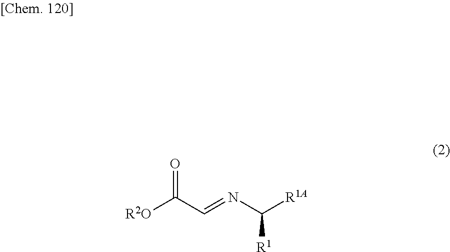

wherein R¹, R^{1A}, and R² are as defined above,
or a pharmaceutically acceptable salt thereof, or a solvate thereof is reacted with 1,3-cyclohexadiene in the presence of a solvent and an acid to prepare a compound of formula (3):

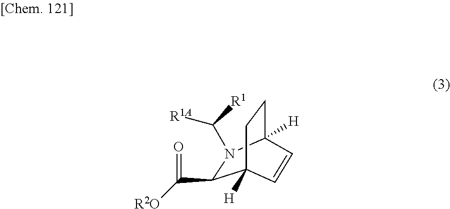

wherein R¹, R^{1A}, and R² are as defined above,
or a pharmaceutically acceptable salt thereof, or a solvate thereof.

### Item A9

The compound of Item A8, or a pharmaceutically acceptable salt thereof, or a solvate thereof, wherein the process further comprises Step C shown below,

### Step C:

a compound of formula (3):

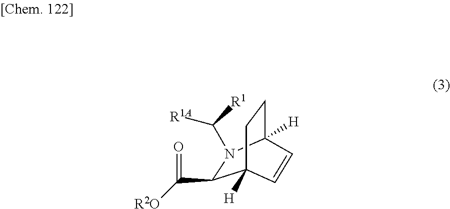

wherein R¹, R^{1A}, and R² are as defined above,
or a pharmaceutically acceptable salt thereof, or a solvate thereof is reacted with borane in the presence of a solvent, and then with a peroxide and a base to prepare a compound of formula (4):

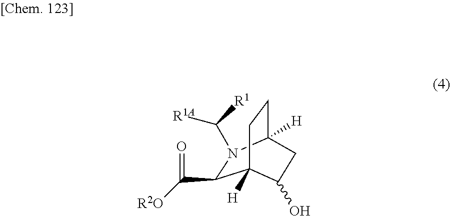

wherein R¹, R^{1A}, and R² are as defined above,
or a pharmaceutically acceptable salt thereof, or a solvate thereof.

### Item A10

The compound of Item A9, or a pharmaceutically acceptable salt thereof, or a solvate thereof, wherein the process further comprises Step D shown below,

### Step D:

a compound of formula (4):

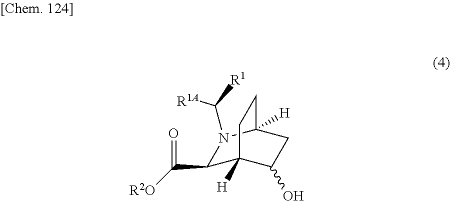

wherein R¹, R^{1A}, and R² are as defined above,
or a pharmaceutically acceptable salt thereof, or a solvate thereof is reacted with hydrogen in the presence of a catalyst for deprotecting the protecting group on the amino group to prepare a compound of formula (5):

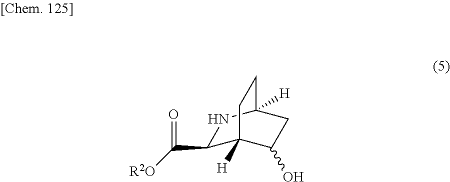

wherein R² is as defined above,
or a pharmaceutically acceptable salt thereof, or a solvate thereof.

### Item A11

The compound of Item A10, or a pharmaceutically acceptable salt thereof, or a solvate thereof, wherein the process further comprises Step E shown below,

### Step E:

a compound of formula (5):

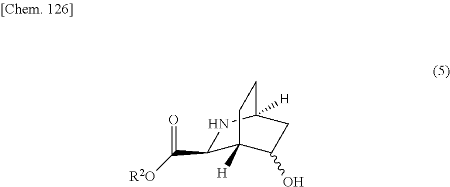

wherein R² is as defined above,
or a pharmaceutically acceptable salt thereof, or a solvate thereof is hydrolyzed of its ester group to prepare a compound of formula (6):

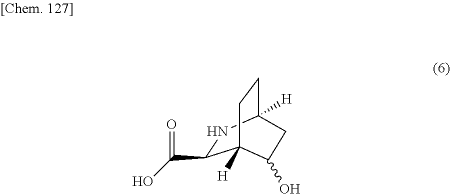 [SMILES: O=C(O)[C@H]1N[C@H]2CC[C@@H]1C(O)C2]

or a pharmaceutically acceptable salt thereof, or a solvate thereof.

### Item A12

The compound of Item A11, or a pharmaceutically acceptable salt thereof, or a solvate thereof, wherein the process further comprises Step F shown below,

### Step F

a compound of formula (6):

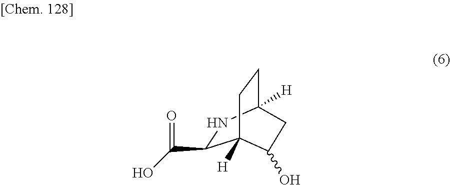 [SMILES: O=C(O)[C@H]1N[C@H]2CC[C@@H]1C(O)C2]

or a pharmaceutically acceptable salt thereof, or a solvate thereof is protected of its amino group to prepare a compound of formula (7):

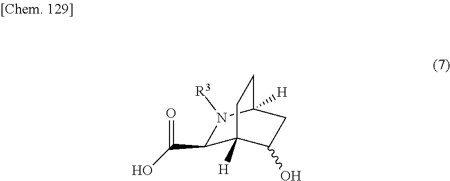

wherein R³ is as defined above,
or a pharmaceutically acceptable salt thereof, or a solvate thereof.

### Item A13

A compound of formula (12):

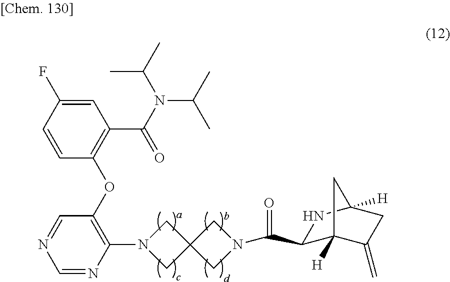

wherein a, b, c, and d are independently 1 or 2,
or a pharmaceutically acceptable salt thereof, or a solvate thereof, which is prepared by a process comprising Step M shown below,

### Step M:

a compound of formula (2):

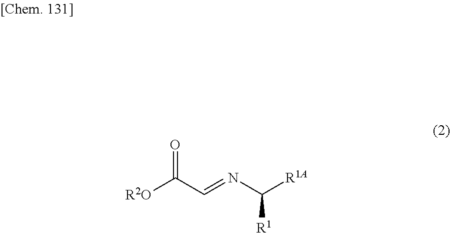

wherein R¹, R^{1A}, and R² are as defined above,
or a pharmaceutically acceptable salt thereof, or a solvate thereof is reacted with a compound of formula (2a):

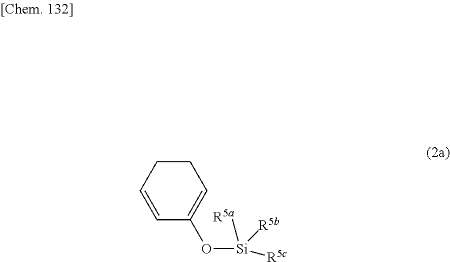

wherein R^{5a}, R^{5b}, and R^{5c} are independently C₁₋₆ alkyl or C₆₋₁₀ aryl,
or a pharmaceutically acceptable salt thereof, or a solvate thereof under an acidic condition in the presence of a solvent to prepare a compound of formula (13):

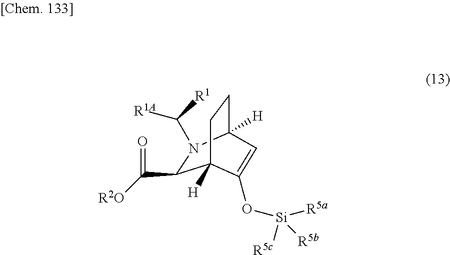

wherein R¹, R^{1A}, R², R^{5a}, R^{5b}, and R^{5c} are as defined above,
or a pharmaceutically acceptable salt thereof, or a solvate thereof.

### Item A14

The compound of Item A13, or a pharmaceutically acceptable salt thereof, or a solvate thereof, wherein the process further comprises Step A shown below,

### Step A:

a compound of formula (1):

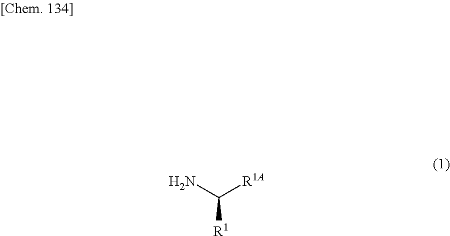

wherein R¹ and R^{1A} are as defined above,
or a pharmaceutically acceptable salt thereof, or a solvate thereof is reacted with a compound of formula (1a):

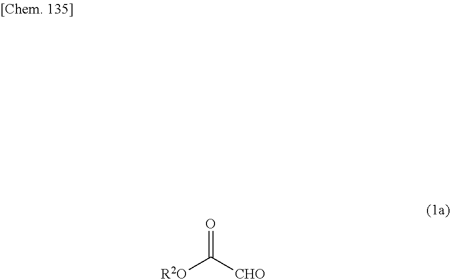

wherein R² is as defined above,
or a pharmaceutically acceptable salt thereof, or a solvate thereof in the presence of a solvent to prepare a compound of formula (2):

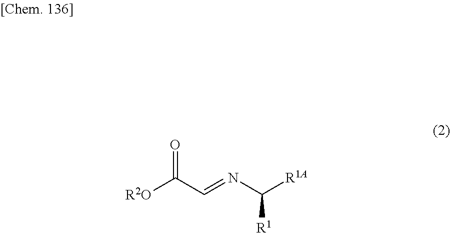

wherein R¹, R^{1A}, and R² are as defined above,
or a pharmaceutically acceptable salt thereof, or a solvate thereof.

### Item A15

The compound of Item A13 or A14, or a pharmaceutically acceptable salt thereof, or a solvate thereof, wherein the process further comprises Step N shown below,

### Step N:

a compound of formula (13):

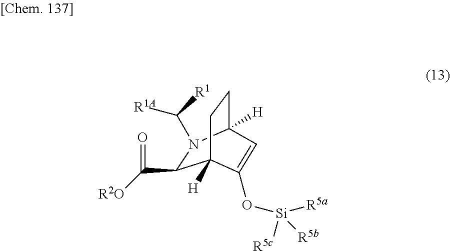

wherein R¹, R^{1A}, R², R^{5a}, R^{5b}, and R^{5c} are as defined above,
or a pharmaceutically acceptable salt thereof, or a solvate thereof is hydrolyzed of its silyl enol ether group to prepare a compound of formula (14):

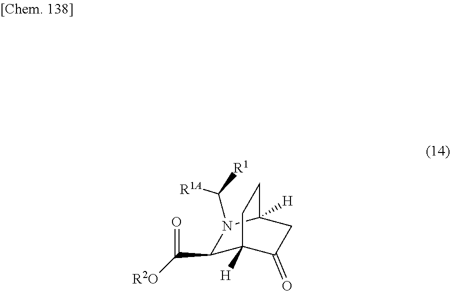

wherein R¹, R^{1A}, and R² are as defined above,
or a pharmaceutically acceptable salt thereof, or a solvate thereof.

### Item A16

The compound of Item A15, or a pharmaceutically acceptable salt thereof, or a solvate thereof, wherein the process further comprises Step O shown below,

### Step O:

a compound of formula (14):

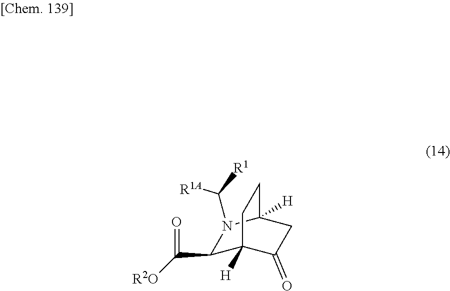

wherein R¹, R^{1A}, and R² are as defined above,
or a pharmaceutically acceptable salt thereof, or a solvate thereof is reacted with hydrogen in the presence of a catalyst for deprotecting the protecting group on the amino group to prepare a compound of formula (15):

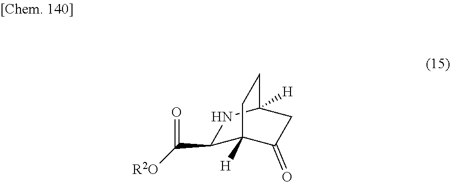

wherein R² is as defined above,
or a pharmaceutically acceptable salt thereof, or a solvate thereof.

### Item A17

The compound of Item A16, or a pharmaceutically acceptable salt thereof, or a solvate thereof, wherein the process further comprises Step P shown below,

### Step P:

a compound of formula (15):

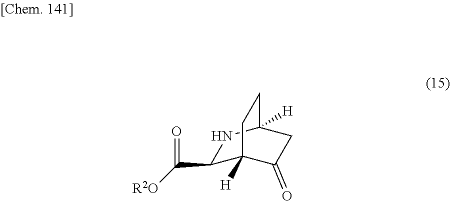

wherein R² is as defined above,
or a pharmaceutically acceptable salt thereof, or a solvate thereof is protected of its amino group to prepare a compound of formula (16):

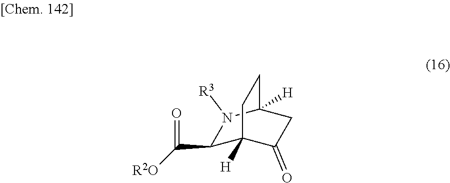

wherein R² and R³ are as defined above,
or a pharmaceutically acceptable salt thereof, or a solvate thereof.

### Item A18

The compound of Item A17, or a pharmaceutically acceptable salt thereof, or a solvate thereof, wherein the process further comprises Step Q shown below,

### Step Q:

a compound of formula (16):

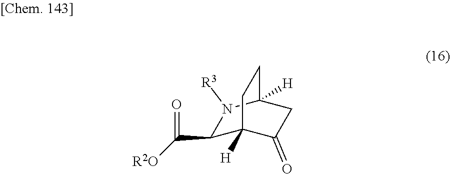

wherein R² and R³ are as defined above,
or a pharmaceutically acceptable salt thereof, or a solvate thereof is hydrolyzed of its ester group to prepare a compound of formula (8):

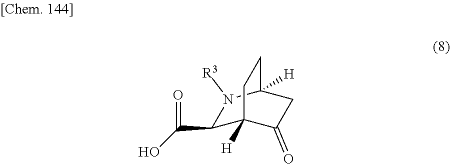

wherein R³ is as defined above,
or a pharmaceutically acceptable salt thereof, or a solvate thereof.

### Item A19

The compound of Item A18, or a pharmaceutically acceptable salt thereof, or a solvate thereof, wherein the process further comprises Step H shown below,

### Step H:

a compound of formula (8):

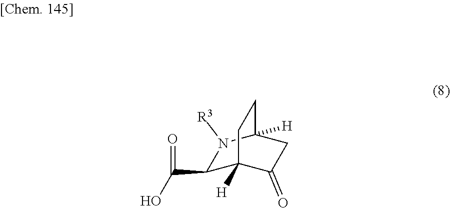

wherein R³ is as defined above,
or a pharmaceutically acceptable salt thereof, or a solvate thereof is reacted with a compound of formula (8a):

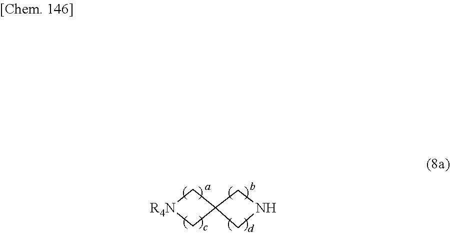

wherein R⁴, a, b, c, and d are as defined above,
or a pharmaceutically acceptable salt thereof, or a solvate thereof in the presence of a solvent and a condensation agent to prepare a compound of formula (9):

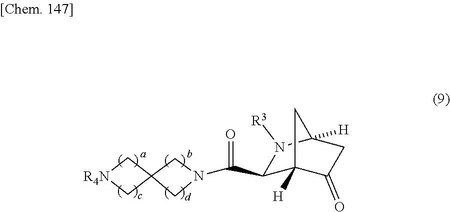

wherein R³, R⁴, a, b, c, and d are as defined above,
or a pharmaceutically acceptable salt thereof, or a solvate thereof.

### Item A20

The compound of Item A19, or a pharmaceutically acceptable salt thereof, or a solvate thereof, wherein the process further comprises Step I shown below,

### Step I:

a compound of formula (9):

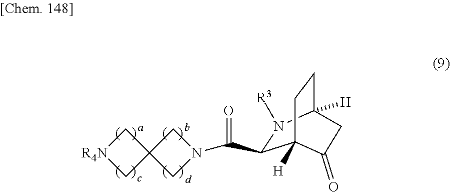

wherein R³, R⁴, a, b, c, and d are as defined above,
or a pharmaceutically acceptable salt thereof, or a solvate thereof is reacted with halogenated methyltriphenylphosphonium and a base in the presence of a solvent to prepare a compound of formula (10):

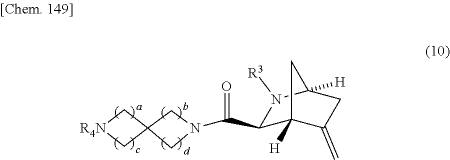

wherein R³, R⁴, a, b, c, and d are as defined above,
or a pharmaceutically acceptable salt thereof, or a solvate thereof.

### Item A21

The compound of Item A20, or a pharmaceutically acceptable salt thereof, or a solvate thereof, wherein the process further comprises Step J shown below,

### Step J:

a compound of formula (10):

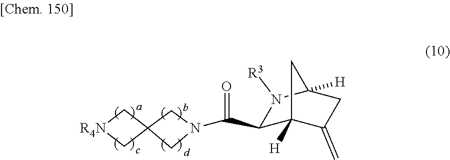

wherein R³, R⁴, a, b, c, and d are as defined above,
or a pharmaceutically acceptable salt thereof, or a solvate thereof is reacted with an acid to prepare a compound of formula (11):

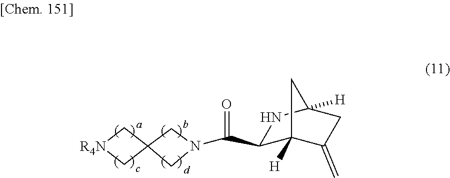

wherein a, b, c, and d are as defined above,
or a pharmaceutically acceptable salt thereof, or a solvate thereof.

### Item A22

The compound of Item A21, or a pharmaceutically acceptable salt thereof, or a solvate thereof, wherein the process further comprises Step K shown below,

### Step K:

a compound of formula (11):

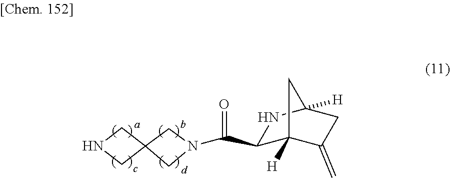

wherein a, b, c, and d are as defined above,
or a pharmaceutically acceptable salt thereof, or a solvate thereof is reacted with a compound of formula (11a):

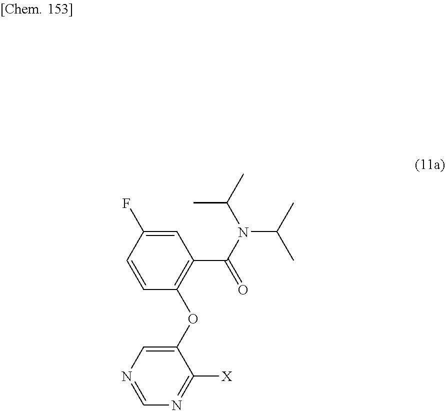

wherein X is fluorine, chlorine, bromine, iodine, p-toluenesulfonyl group, or methanesulfonyl group,
or a pharmaceutically acceptable salt thereof, or a solvate thereof in the presence of a solvent and a base to prepare a compound of formula (12):

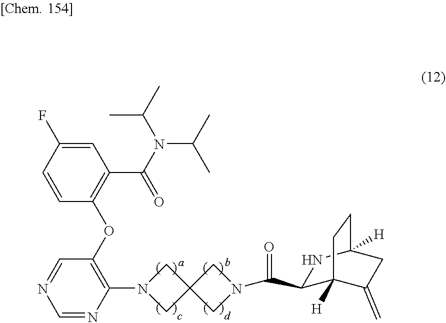

wherein a, b, c, and d are as defined above,
or a pharmaceutically acceptable salt thereof, or a solvate thereof.

### Item A23

The compound of Item A22, or a pharmaceutically acceptable salt thereof, or a solvate thereof, wherein the process further comprises Step L shown below,

### Step L:

a compound of formula (11):

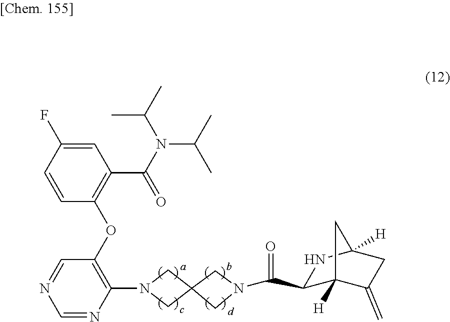

wherein a, b, c, and d are as defined above,
or a pharmaceutically acceptable salt thereof, or a solvate thereof is reacted with L(+)-tartaric acid in the presence of a solvent to prepare a compound of formula (12a):

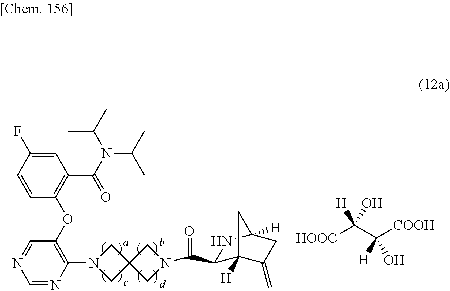

wherein a, b, c, and d are as defined above,
or a solvate thereof.

### Item A24

A compound of formula (12):

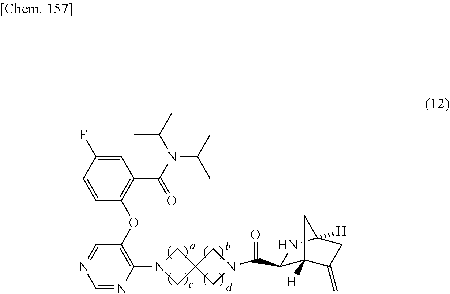

wherein a, b, c, and d are independently 1 or 2,
or a pharmaceutically acceptable salt thereof, or a solvate thereof, which is prepared by a process comprising Step H shown below,

### Step H:

a compound of formula (8):

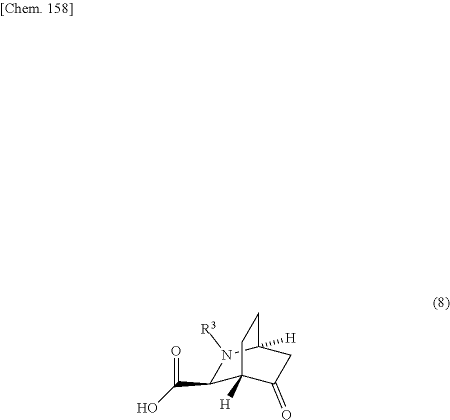

wherein R³ is as defined above,
or a pharmaceutically acceptable salt thereof, or a solvate thereof is reacted with a compound of formula (8a):

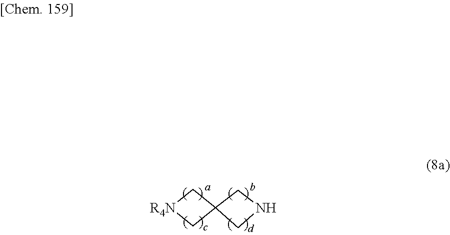

wherein R⁴, a, b, c, and d are as defined above,
or a pharmaceutically acceptable salt thereof, or a solvate thereof in the presence of a solvent and a condensation agent to prepare a compound of formula (9):

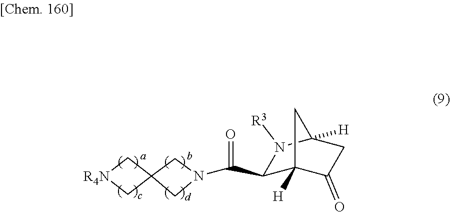

wherein R³, R⁴, a, b, c, and d are as defined above,
or a pharmaceutically acceptable salt thereof, or a solvate thereof.

### Item A25

The compound of Item A24, or a pharmaceutically acceptable salt thereof, or a solvate thereof, wherein the process further comprises Step I shown below,

### Step I:

a compound of formula (9)

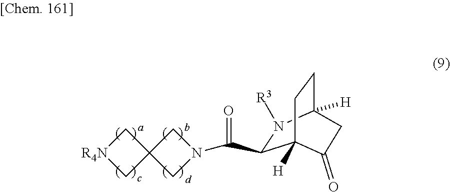

wherein R³, R⁴, a, b, c, and d are as defined above,
or a pharmaceutically acceptable salt thereof, or a solvate thereof is reacted with halogenated methyltriphenylphosphonium, trialkylsilylmethyl anion, or methylsulfone derivative, in the presence of a solvent and a base to prepare a compound of formula (10):

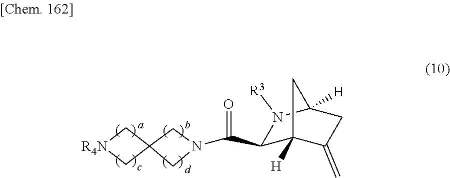

wherein R³, R⁴, a, b, c, and d are as defined above,
or a pharmaceutically acceptable salt thereof, or a solvate thereof.

### Item A26

The compound of Item A25, or a pharmaceutically acceptable salt thereof, or a solvate thereof, wherein the process further comprises Step J shown below,

### Step J:

a compound of formula (10):

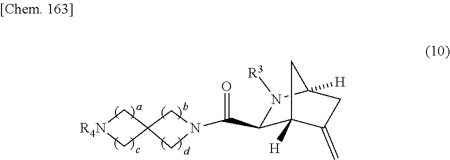

wherein R³, R⁴, a, b, c, and d are as defined above,
or a pharmaceutically acceptable salt thereof, or a solvate thereof is reacted with an acid to prepare a compound of formula (11):

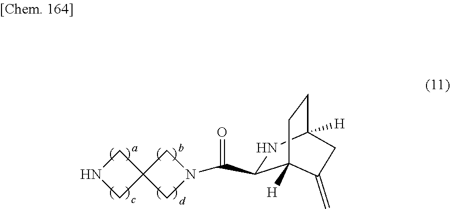

wherein a, b, c, and d are as defined above,
or a pharmaceutically acceptable salt thereof, or a solvate thereof.

### Item A27

The compound of Item A26, or a pharmaceutically acceptable salt thereof, or a solvate thereof, wherein the process further comprises Step K shown below,

### Step K:

a compound of formula (11):

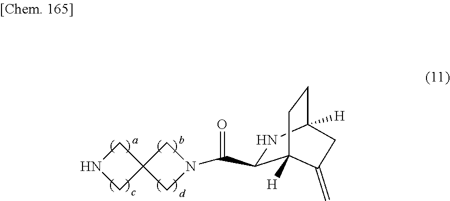

wherein a, b, c, and d are as defined above,
or a pharmaceutically acceptable salt thereof, or a solvate thereof is reacted with a compound of formula (11a):

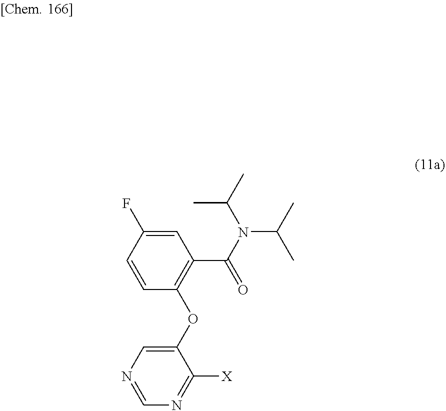

wherein X is fluorine, chlorine, bromine, iodine, p-toluenesulfonyl group, or methanesulfonyl group,
or a pharmaceutically acceptable salt thereof, or a solvate thereof in the presence of a solvent and a base to prepare a compound of formula (12):

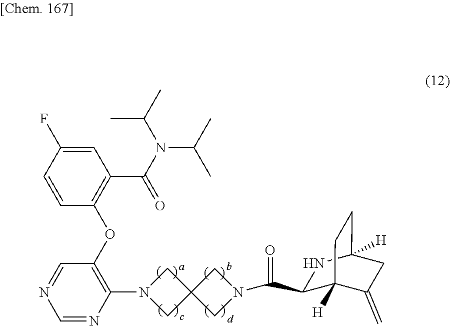

wherein a, b, c, and d are as defined above,
or a pharmaceutically acceptable salt thereof, or a solvate thereof.

### Item A28

The compound of Item A27, or a pharmaceutically acceptable salt thereof, or a solvate thereof, wherein the process further comprises Step L shown below,

### Step L:

a compound of formula (12):

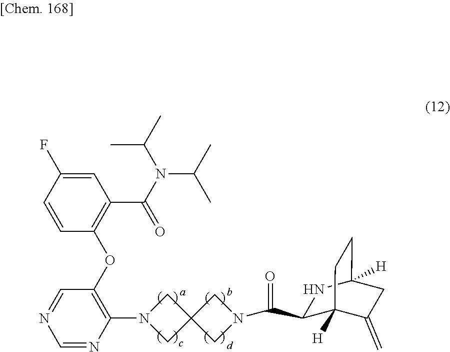

wherein a, b, c, and d are as defined above,
or a pharmaceutically acceptable salt thereof, or a solvate thereof is reacted with L(+)-tartaric acid in the presence of a solvent to prepare a compound of formula (12a):

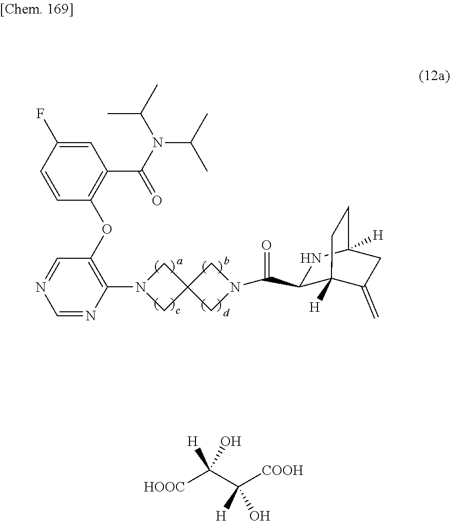

wherein a, b, c, and d are as defined above,
or a solvate thereof.

### Item B1

A mixture comprising 5-fluoro-2-[(4-{7-[(1S,3S,4R)-5-methylidene-2-azabicyclo[2.2.2]octane-3-carbonyl]-2,7-diazaspiro[3.5]nonan-2-yl}pyrimidin-5-yl)oxy]-N,N-di(propan-2-yl)benzamide or a pharmaceutically acceptable salt thereof, or a solvate thereof, and Compound A (5-fluoro-2-[(4-{7-[(1S,3S,4S)-5-hydroxy-5-methyl-2-azabicyclo[2.2.2]octane-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof.

### Item B2

The mixture of Item B1, wherein Compound A (5-fluoro-2-[(4-{7-[(1S,3S,4S)-5-hydroxy-5-methyl-2-azabicyclo[2.2.2]octane-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof is present in amount of about 0.15% or less of the mixture as determined by high performance liquid chromatography (HPLC).

### Item B3

The mixture of Item B1, wherein Compound A (5-fluoro-2-[(4-{7-[(1S,3S,4S)-5-hydroxy-5-methyl-2-azabicyclo[2.2.2]octane-3-carbonyl]-2,7-diazaspiro[3.5]nonan-2-yl}pyrimidin-5-yl)oxy]-N, N-di(propan-2-yl)benzamide) or a pharmaceutically acceptable salt thereof, or a solvate thereof is present in amount of about 0.1% or less of the mixture as determined by high performance liquid chromatography (HPLC).

### Item B4

The mixture of Item B1, wherein Compound A (5-fluoro-2-[(4-{7-[(1S,3S,4S)-5-hydroxy-5-methyl-2-azabicyclo[2.2.2]octane-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof is present in amount of about 0.05% or less of the mixture as determined by high performance liquid chromatography (HPLC).

### Item B5

The mixture according to any one of Items B1 to B4, further comprising Compound B (2-[(4-{(1S,3S,4R)-3-[2-(5-{2-[di(propan-2-yl)carbamoyl]-4-fluorophenoxy}pyrimidin-4-yl)-2,7-diazaspiro[3.5]nonane-7-carbonyl]-5-methylidene-2-azabicyclo[2.2.2]octan-2-yl}pyrimidin-5-yl)oxy]-5-fluoro-N,N-di(propan-2-yl)benzamide) or a pharmaceutically acceptable salt thereof, or a solvate thereof.

### Item B6

The mixture according to Item B5, wherein Compound B (2-[(4-{(1S,3S,4R)-3-[2-(5-{2-[di(propan-2-yl)carbamoyl]-4-fluorophenoxy}pyrimidin-4-yl)-2,7-diazaspiro[3.5]nonane-7-carbonyl]-5-methylidene-2-azabicyclo[2.2.2]octan-2-yl}pyrimidin-5-yl)oxy]-5-fluoro-N,N-di(propan-2-yl)benzamide) or a pharmaceutically acceptable salt thereof, or a solvate thereof is present in amount of about 0.15% or less of the mixture as determined by high performance liquid chromatography (HPLC).

### Item B7

The mixture according to Item B5, wherein Compound B (2-[(4-{(1S,3S,4R)-3-[2-(5-{2-[di(propan-2-yl)carbamoyl]-4-fluorophenoxy}pyrimidin-4-yl)-2,7-diazaspiro[3.5]nonane-7-carbonyl]-5-methylidene-2-azabicyclo[2.2.2]octan-2-yl}pyrimidin-5-yl)oxy]-5-fluoro-N,N-di(propan-2-yl)benzamide) or a pharmaceutically acceptable salt thereof, or a solvate thereof is present in amount of about 0.1% or less of the mixture as determined by high performance liquid chromatography (HPLC).

### Item B8

The mixture according to Item B5, wherein Compound B (2-[(4-{(1S,3S,4R)-3-[2-(5-{2-[di(propan-2-yl)carbamoyl]-4-fluorophenoxy}pyrimidin-4-yl)-2,7-diazaspiro[3.5]nonane-7-carbonyl]-5-methylidene-2-azabicyclo[2.2.2]octan-2-yl}pyrimidin-5-yl)oxy]-5-fluoro-N,N-di(propan-2-yl)benzamide) or a pharmaceutically acceptable salt thereof, or a solvate thereof is present in amount of about 0.05% or less of the mixture as determined by high performance liquid chromatography (HPLC).

### Item B9

The mixture according to Item B5, further comprising Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof.

### Item B10

The mixture according to Item B9, wherein Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof is present in amount of about 0.15% or less of the mixture as determined by high performance liquid chromatography (HPLC).

### Item B11

The mixture according to Item B9, wherein Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof is present in amount of about 0.1% or less of the mixture as determined by high performance liquid chromatography (HPLC).

### Item B12

The mixture according to Item B9, wherein Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof is present in amount of about 0.06% or less of the mixture as determined by high performance liquid chromatography (HPLC).

### Item B13

The mixture according to any one of Items B1 to B4, further comprising Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof.

### Item B14

The mixture according to Item B13, wherein Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof is present in amount of about 0.15% or less of the mixture as determined by high performance liquid chromatography (HPLC).

### Item B15

The mixture according to Item B13, wherein Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof is present in amount of about 0.1% or less of the mixture as determined by high performance liquid chromatography (HPLC).

### Item B16

The mixture according to Item B13, wherein Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof is present in amount of about 0.06% or less of the mixture as determined by high performance liquid chromatography (HPLC).

### Item B17

A mixture comprising 5-fluoro-2-[(4-{7-[(1S,3S,4R)-5-methylidene-2-azabicyclo[2.2.2]octane-3-carbonyl]-2,7-diazaspiro[3.5]nonan-2-yl}pyrimidin-5-yl)oxy]-N,N-di(propan-2-yl)benzamide or a pharmaceutically acceptable salt thereof, or a solvate thereof, and Compound B (2-[(4-{(1S,3S,4R)-3-[2-(5-{2-[di(propan-2-yl)carbamoyl]-4-fluorophenoxy}pyrimidin-4-yl)-2,7-diazaspiro[3.5]nonane-7-carbonyl]-5-methylidene-2-azabicyclo[2.2.2]octan-2-yl}pyrimidin-5-yl)oxy]-5-fluoro-N,N-di(propan-2-yl)benzamide) or a pharmaceutically acceptable salt thereof, or a solvate thereof.

### Item B18

The mixture of Item B17, wherein Compound B (2-[(4-{(1S,3S,4R)-3-[2-(5-{2-[di(propan-2-yl)carbamoyl]-4-fluorophenoxy}pyrimidin-4-yl)-2,7-diazaspiro[3.5]nonane-7-carbonyl]-5-methylidene-2-azabicyclo[2.2.2]octan-2-yl}pyrimidin-5-yl)oxy]-5-fluoro-N,N-di(propan-2-yl)benzamide) or a pharmaceutically acceptable salt thereof, or a solvate thereof is present in amount of about 0.15% or less of the mixture as determined by high performance liquid chromatography (HPLC).

### Item B19

The mixture of Item B17, wherein Compound B (2-[(4-{(1S,3S,4R)-3-[2-(5-{2-[di(propan-2-yl)carbamoyl]-4-fluorophenoxy}pyrimidin-4-yl)-2,7-diazaspiro[3.5]nonane-7-carbonyl]-5-methylidene-2-azabicyclo[2.2.2]octan-2-yl}pyrimidin-5-yl)oxy]-5-fluoro-N,N-di(propan-2-yl)benzamide) or a pharmaceutically acceptable salt thereof, or a solvate thereof is present in amount of about 0.1% or less of the mixture as determined by high performance liquid chromatography (HPLC).

### Item B20

The mixture of Item B17, wherein Compound B (2-[(4-{(1S,3S,4R)-3-[2-(5-{2-[di(propan-2-yl)carbamoyl]-4-fluorophenoxy}pyrimidin-4-yl)-2,7-diazaspiro[3.5]nonane-7-carbonyl]-5-methylidene-2-azabicyclo[2.2.2]octan-2-yl}pyrimidin-5-yl)oxy]-5-fluoro-N,N-di(propan-2-yl)benzamide) or a pharmaceutically acceptable salt thereof, or a solvate thereof is present in amount of about 0.05% or less of the mixture as determined by high performance liquid chromatography (HPLC).

### Item B21

The mixture according to any one of Items B17 to B20, further comprising Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof.

### Item B22

The mixture according to Item B21, wherein Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof is present in amount of about 0.15% or less of the mixture as determined by high performance liquid chromatography (HPLC).

### Item B23

The mixture according to Item B21, wherein Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof is present in amount of about 0.1% or less of the mixture as determined by high performance liquid chromatography (HPLC).

### Item B24

The mixture according to Item B21, wherein Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof is present in amount of about 0.06% or less of the mixture as determined by high performance liquid chromatography (HPLC).

### Item B25

A mixture comprising 5-fluoro-2-[(4-{7-[(1S,3S,4R)-5-methylidene-2-azabicyclo[2.2.2]octane-3-carbonyl]-2,7-diazaspiro[3.5]nonan-2-yl}pyrimidin-5-yl)oxy]-N,N-di(propan-2-yl)benzamide or a pharmaceutically acceptable salt thereof, or a solvate thereof, and Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof.

### Item B26

The mixture of Item B25, wherein Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof is present in amount of about 0.15% or less of the mixture as determined by high performance liquid chromatography (HPLC).

### Item B27

The mixture of Item B25, wherein Compound C (5-fluoro-2-[(4-{7-[(1S,3S,4R)-5-methyl-2-azabicyclo[2.2.2]oct-5-ene-3-carbonyl)-2,7-diazaspiro[3.5]nonan-2-yl}pyrimidin-5-yl)oxy]-N,N-di(propan-2-yl)benzamide) or a pharmaceutically acceptable salt thereof, or a solvate thereof is present in amount of about 0.1% or less of the mixture as determined by high performance liquid chromatography (HPLC).

### Item B28

The mixture of Item B25, wherein Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof is present in amount of about 0.06% or less of the mixture as determined by high performance liquid chromatography (HPLC).

It is contemplated in the present disclosure that one or more features described above may be provided in the explicit combinations, as well as further combinations in addition to the explicit ones. Still further embodiments and advantages of the present disclosure will be appreciated by a skilled person upon reading and understanding the following detailed description, if necessary.

### Effect of Invention

In one embodiment, according to Process 1 in the present disclosure, intermediate (8) can be prepared in high yield and high purity with fewer protection and deprotection steps than by known processes. Furthermore, optically-active azabicyclo ring derivative (12) useful as a pharmaceutical can be prepared from intermediate (8) in only four steps in high yield and high purity.

In one embodiment, according to Process 2 in the present disclosure, intermediate (8) can be prepared using intermediate (2a) in fewer steps than Process 1, and optically-active azabicyclo ring derivative (12) useful as a pharmaceutical can be prepared from intermediate (8) in only four steps in high yield and high purity.

### DESCRIPTION OF EMBODIMENTS

Hereinafter, the present disclosure is described showing the best mode. It should be understood that throughout the present specification, expressions in the singular also include the concept of the plural, unless otherwise indicated.

Thus, articles in the singular (e.g. “a”, “an”, “the”, etc. in the English language) should be understood to include their plural forms as well, unless otherwise indicated. Also, it should be understood that the terms used in the present specification have the meanings commonly used in the relevant field, unless otherwise indicated. Thus, unless otherwise defined, all technical and scientific terms used herein have the same meaning as commonly understood by a skilled person in the art to which the present disclosure belongs. In case of conflict, the present specification (including definitions) will control.

The compounds of the present disclosure may also exist in the form of solvates (e.g. hydrates), thus solvates (e.g., hydrate) of the compounds of formulae (1), (1a), (2), (2a), (3), (4), (5), (6), (7), (8), (8a), (9), (10), (11), (11a), (12), (12a), (13), (14), (15), and (16), or tautomers thereof, or of optionally pharmaceutically acceptable salts thereof, are also encompassed in the present compounds.

The compounds of formulae (1), (1a), (2), (2a), (3), (4), (5), (6), (7), (8), (8a), (9), (10), (11), (11a), (12), (12a), (13), (14), (15), and (16) may have one or more asymmetric carbon atoms, or may exhibit geometric isomerism or axial chirality, i.e., which may exist as several stereoisomers.

In the present disclosure, these stereoisomers, mixtures thereof and racemates thereof are also encompassed in the present compounds.

Further, the compounds of formulae (1), (1a), (2), (2a), (3), (4), (5), (6), (7), (8), (8a), (9), (10), (11), (11a), (12), (12a), (13), (14), (15), and (16), wherein one or more 1H (hydrogen) is replaced by ²H (D: deuterated hydrogen), are also encompassed in the compounds of formulae (1), (1a), (2), (2a), (3), (4), (5), (6), (7), (8), (8a), (9), (10), (11), (11a), (12), (12a), (13), (14), (15), and (16).

Crystals of the compounds of formulae (1), (1a), (2), (2a), (3), (4), (5), (6), (7), (8), (8a), (9), (10), (11), (11a), (12), (12a), (13), (14), (15), and (16), or tautomers thereof, or optionally pharmaceutically acceptable salts thereof may have crystalline polymorphism, which are also encompassed in the present compounds.

In the present specification, the number of carbon atoms in the definition of “substituent” may be expressed as, for example, “C₁₋₆”. In detail, the expression “C₁₋₆ alkyl” is the same meaning as an alkyl group having 1 to 6 carbon atoms. In addition, substituents which are not accompanied with explicit term of “may be substituted”, “optionally-substituted”or “substituted” in the present specification mean “unsubstituted” substituents. For example, “C₁₋₆ alkyl” means “unsubstituted” C₁₋₆ alkyl.

In the present specification, the term “group” means a monovalent group. For example, “alkyl group” means a monovalent saturated hydrocarbon group. And, the term “group” may be omitted in the description of substituents in the present specification.

The terms used herein are explained below.

In the present specification, the number of substituents in the group defined by “may be substituted”, “optionally-substituted” or “substituted” is not particularly limited as long as it can be substituted, which includes 0, 1 or more. And, unless otherwise indicated, the definition of each group also applies to the case that the group is used as a part or a substituent of another group.

In the present specification, the substituents in “optionally-substituted . . . ” can be appropriately selected depending on the group to be substituted. For example, the “optionally-substituted C₁₋₆ alkyl group” means the C₁₋₆ alkyl optionally-substituted at any substitutable position with fluorine atom, chlorine atom, phenyl group, methoxy group, and/or hydroxyl group, which includes methyl group, ethyl group, n-propyl group and isopropyl group, difluoromethyl group, trifluoromethyl group, 2-fluoroethyl group, 2-methoxyethyl group, and benzyl group. Preferably, it is methyl group, ethyl group, difluoromethyl group, trifluoromethyl group, 2-fluoroethyl group, 2-methoxyethyl group, or benzyl group.

The “C₁₋₆ alkyl” means a straight- or branched-chain saturated hydrocarbon group having 1 to 6 carbon atoms, and “C₆ alkyl” means alkyl having 6 carbon atoms. The same is applied to the case of the other carbon numbers. The “C₁₋₆ alkyl” includes preferably “C₁₋₄ alkyl”, more preferably “C₁₋₃ alkyl”. The “C₁₋₃ alkyl” includes, for example, methyl, ethyl, propyl, and 1-methylethyl. The “C₁₋₄ alkyl” includes, for example, butyl, 1,1-dimethylethyl, 1-methylpropyl, 2-methylpropyl, and the like, besides the examples listed in the said “C₁₋₃ alkyl”. “C₁₋₆ alkyl” includes, for example, pentyl, 1,1-dimethylpropyl, 1,2-dimethylpropyl, 1-methylbutyl, 2-methylbutyl, 4-methylpentyl, 3-methylpentyl, 2-methylpentyl, 1-methylpentyl, hexyl, and the like, besides the examples listed in the said “C₁₋₄ alkyl”.

The “C₆₋₁₀ aryl” means an aromatic hydrocarbon ring group having 6 to 10 carbon atoms. The “C₆₋₁₀ aryl” includes, for example, phenyl, 1-naphthyl, and 2-naphthyl.

In the reaction of the present disclosure, if necessary, a phase transfer catalyst such as tetrabutylammonium salt may be added as long as it does not adversely affect the reaction.

The “base” includes both organic and inorganic bases.

The “organic base” includes, for example, sodium tert-butoxide, potassium tert-butoxide, sodium methoxide, triethylamine, N,N,N′,N′-tetramethylethane-1,2-diamine, N,N-dimethylaniline, N,N-diisopropylethylamine, N-methylpyrrolidine, N-methylpiperidine, piperidine, 1,4-diazabicyclo[2.2.2]octane, 1,5-diazabicyclo[4.3.0]-5-nonene, 1,8-diazabicyclo[5.4.0]-7-undecene, N-methylmorpholine, diazabicycloundecene, methylamine, ethylamine, diisopropylamine, 4-dimethylaminopyridine, 2,6-lutidine, pyrimidine, and pyridine, or a mixture thereof, but not limited thereto. More preferably, it includes sodium tert-butoxide, potassium tert-butoxide, triethylamine, diisopropylethylamine, N,N,N′,N′-tetramethylethane-1,2-diamine, 1,4-diazabicyclo[2.2.2]octane, N-methylpiperidine, pyrimidine, and pyridine. Even more preferably, it includes sodium tert-butoxide, potassium tert-butoxide, triethylamine, diisopropylethylamine, N,N,N′,N′-tetramethylethane-1,2-diamine, 1,4-diazabicyclo[2.2.2]octane, N-methylpiperidine, pyrimidine, and pyridine. The most preferably, it includes sodium tert-butoxide, potassium tert-butoxide, triethylamine, and N,N-diisopropylethylamine.

The “inorganic base” includes, for example, ammonia, lithium hydroxide, sodium hydroxide, calcium hydroxide, barium hydroxide, sodium carbonate, sodium hydrogen carbonate, potassium carbonate, and cesium carbonate, or a mixture thereof, but not limited thereto. Preferably, it includes lithium hydroxide, sodium hydroxide, sodium carbonate, potassium carbonate, sodium hydrogen carbonate, and cesium carbonate. More preferably, it includes sodium carbonate, potassium carbonate, sodium hydrogen carbonate, and cesium carbonate. The most preferably, it includes potassium carbonate.

The “acid” includes both organic and inorganic acids.

The “organic acid” includes organic acids and Lewis acids, for example, formic acid, acetic acid, trifluoroacetic acid, p-toluenesulfonic acid, methanesulfonic acid, trichloroacetic acid, dichloroacetic acid, difluoroacetic acid, citric acid, oxalic acid, tartaric acid, 1,1′-bi(2-naphthol), 1,1′-binaphthyl-2,2′-diyl hydrogenphosphate, 3,3′-bis(2,4,6-triisopropylphenyl)-1,1′-binaphthyl-2,2′-diyl hydrogenphosphate, 3,3′-bis(triphenylsilyl)-1,1′-binaphthyl-2,2′-diyl hydrogenphosphate, boron trifluoride-ethyl ether complex, trimethylsilyl chloride, diethylalminum chloride, zinc bromide, tetrachlorotitanium, and magnesium bromide ethyl etherate, or a mixture thereof, but not limited thereto. More preferably, it includes trifluoroacetic acid, and boron trifluoride-ethyl ether complex, or a mixture thereof.

The “inorganic acid” includes, for example, hydrochloric acid, hydrobromic acid, nitric acid, phosphoric acid, and sulfuric acid, or a mixture thereof, but not limited thereto. Preferably, it includes hydrochloric acid.

The “alcohol solvent” means a solvent that is a compound having one or more hydroxyl groups in the molecule, is liquid at reaction temperature, and has the property of dissolving or dispersing a reaction substrate.

The “alcohol solvent” includes, for example, methanol, ethanol, 1-propanol, 2-propanol, 1-butanol, 2-butanol, 3-methyl-1-butanol, 2-methyl-1-propanol, and tert-butyl alcohol. Preferably, it includes methanol, ethanol, and 2-propanol. More preferably, it includes ethanol.

The “amide solvent” means a solvent that is a compound having one or more amide bonds in the molecule, is liquid at reaction temperature, and has the property of dissolving or dispersing a reaction substrate. The “amide solvent” includes, for example, N,N-dimethylacetamide, N,N-dimethylformamide, N,N-diethylformamide, N-methyl-2-pyrrolidone, tetramethylurea, and hexamethylphosphoric triamide. The “amide solvent” includes, preferably, N,N-dimethylformamide and N-methyl-2-pyrrolidone. More preferably, it includes N,N-dimethylformamide.

The “halogen solvent” means a solvent that is a compound having one or more halogen atoms in the molecule, is liquid at reaction temperature, and has the property of dissolving or dispersing a reaction substrate. The “halogen solvent” includes, for example, dichloromethane, chloroform, 1,2-dichloroethane, and chlorobenzene. The “halogen solvent” includes, preferably, dichloromethane.

The “ether solvent” means a solvent that is a compound having one or more ether bonds in the molecule, is liquid at reaction temperature, and has the property of dissolving or dispersing a reaction substrate. The “ether solvent” includes, for example, diethyl ether, tetrahydrofuran, methyl tert-butyl ether, and 1,4-dioxane. The “ether solvent” includes, preferably, tetrahydrofuran, methyl tert-butyl ether, and 1,4-dioxane.

The “ester solvent” means a solvent that is a compound having one or more ester bonds in the molecule, is liquid at reaction temperature, and has the property of dissolving or dispersing a reaction substrate. The “ester solvent” includes, for example, ethyl acetate and isopropyl acetate. The “ester solvent” includes, preferably, ethyl acetate.

The “protecting group for an amino group” includes, for example, protecting groups described in Protective Groups in Organic Synthesis (Theodora W. Greene, Peter G. M. Wuts, published by John Wiley & Sons, Inc., 1999). The “protecting group for an amino group” includes, for example, tert-butoxycarbonyl group and benzyloxycarbonyl group. Preferably, it includes tert-butoxycarbonyl group.

The “reagent for protecting amino group” means a reagent used for protecting an amino group. The “reagent for protecting amino group” includes, for example, di-tert-butyl dicarbonate and benzyl chloroformate. Preferably, it includes di-tert-butyl dicarbonate.

The “condensation agent” means a reagent for condensing a carboxyl group and an amino group by dehydration to form an amide group. The “condensation agent” includes, for example, 1-hydroxybenzotriazole, 1-ethyl-3-(3-dimethylaminopropyl)carbodiimide hydrochloride, and a mixture thereof, but not limited thereto. The “condensation agent” includes, preferably, a mixture of 1-hydroxybenzotriazole and 1-ethyl-3-(3-dimethylaminopropyl)carbodiimide hydrochloride.

The “halogenated methyltriphenylphosphonium” includes, for example, methyltriphenylphosphonium bromide and methyltriphenylphosphonium iodide. The “halogenated methyltriphenylphosphonium” includes, preferably, methyltriphenylphosphonium bromide.

The “trialkylsilylmethyl anion” includes, for example, (trimethylsilyl)methyllithium and (trimethylsilyl)methylmagnesium. The “(trialkylsilyl)methyllithium” includes, preferably, (trimethylsilyl)methyllithium.

The “methylsulfone derivative” includes, for example, 1-methyl-2-(methylsulfonyl)benzimidazole and 2-(methylsulfonyl)benzothiazole. The “methylsulfone derivative” includes, preferably, 1-methyl-2-(methylsulfonyl)benzimidazole.

The “peroxide” includes, for example, hydrogen peroxide, cumene hydroperoxide, benzoyl peroxide, peracetic acid and sodium peroxoborate, which includes hydrate thereof. The “peroxide” includes, preferably, hydrogen peroxide and sodium peroxoborate, which includes hydrate thereof. The “peroxide” includes, more preferably, sodium peroxoborate, which includes hydrate thereof.

The “borane” includes, for example, borane and 9-borabicyclo[3.3.1]nonane, and also includes those dimerized and those complexed with a solvent. The “borane” includes, preferably, borane and borane-tetrahydrofuran complex.

The “oxidation agent” includes, for example, 1-methyl-2-azaadamantane-N-oxyl, 2-hydroxy-2-azaadamantane, 9-azanoradamantan-N-oxyl, 1,1,1-triacetoxy-1,1-dihydro-1,2-benziodoxol-3-(1H)-one, potassium 2-iodo-5-methylbenzenesulfonate, “a combination of DMSO and oxalyl chloride”, acetic anhydride, sulfur trioxide-pyridine-complex, N,N′-dicyclohexylcarbodiimide, chromium trioxide, tetrapropylammonium perruthenate, sodium hypochlorite pentahydrate, 2,2,6,6-tetramethylpiperidine 1-oxyl, and sodium periodate. The “oxidation agent” includes, preferably, 2,2,6,6-tetramethylpiperidine 1-oxyl and sodium periodate, and more preferably 2,2,6,6-tetramethylpiperidine 1-oxyl.

The “re-oxidation agent” includes, for example, oxygen, oxone, morpholine N-oxide, trichloroisocyanuric acid, sodium hypochlorite, and iodobenzene diacetate. The “re-oxidation agent” includes, preferably, trichloroisocyanuric acid.

In the present specification, the term “at least one independently selected from . . . ” which is used in the expressions of solvents, etc. means that when two or more of these options are selected, the two or more options include a mixture thereof.

As used herein, unless otherwise indicated, the term “pharmaceutically acceptable salt” means a salt prepared from a pharmaceutically acceptable acid which includes inorganic and organic acids. And, “optionally pharmaceutically acceptable salts thereof” means that it can be a pharmaceutically acceptable salt at an optional time, for example, in the case of preparing an intermediate, it means that a pharmaceutically unacceptable salt can be used until a certain step. The pharmaceutically acceptable salt includes, for example, acetic acid, alginic acid, anthranilic acid, benzenesulfonic acid, benzoic acid, camphorsulfonic acid, citric acid, ethenesulfonic acid, formic acid, fumaric acid, furoic acid, gluconic acid, glutamic acid, glucorenic acid, galacturonic acid, glycidic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phenylacetic acid, propionic acid, phosphoric acid, salicylic acid, stearic acid, succinic acid, sulfanilic acid, sulfuric acid, tartaric acid, and p-toluenesulfonic acid, but not limited thereto.

The “purification” refers to any act of increasing the purity of a desired substance and making the concentration of substances other than the desired substance lower than that of before the act of purification. The “purification” may be done with various methods such as precipitation, recrystallization, sublimation, distillation, solvent extraction, use of molecular sieves, and application of various chromatographies. The purification does not include filtration using filter paper, Celite, etc.

In the compounds of formulae (1), (1a), (2), (2a), (3), (4), (5), (7), (8), (8a), (9), (10), (11), (11a), (12), (12a), (13), (14), (15), and (16) which are disclosed herein, preferred embodiments of R¹, R^{1A}, R², R³, R⁴, R^{5a}, R^{5b}, R^{5c}, a, b, c, and d are as follows, but not limited thereto.

Preferred embodiment of R¹ includes C₁₋₃ alkyl.

More preferred embodiment of R¹ includes methyl group.

Preferred embodiment of R^{1A} includes C₆₋₁₀ aryl.

More preferred embodiment of R^{1A} includes phenyl group.

Preferred embodiment of R² includes C₁₋₃ alkyl.

More preferred embodiment of R² includes ethyl group.

Preferred embodiment of R³ includes tert-butoxycarbonyl group and benzyloxycarbonyl group.

More preferred embodiment of R³ includes tert-butoxycarbonyl group.

Preferred embodiment of R⁴ includes tert-butoxycarbonyl group and benzyloxycarbonyl group.

More preferred embodiment of R⁴ includes tert-butoxycarbonyl group.

Preferred embodiments of R^{5a}, R^{5b}, and R^{5c} independently include C₁₋₄ alkyl and phenyl group.

More preferred embodiments of R^{5a}, R^{5b}, and R^{5c} independently include methyl group and phenyl group.

Even more preferred embodiments of R^{5a}, R^{5b}, and R^{5c} include methyl group.

Preferred embodiments of a, b, c, and d independently include 1 and 2.

Preferred embodiments of a and c independently include 1 and 2.

More preferred embodiments of a and c include 1.

Preferred embodiments of b and d independently include 1 and 2.

More preferred embodiments of b and d include 2.

Preferred embodiment of X includes fluorine, chlorine, bromine, iodine, p-toluenesulfonyl group, and methanesulfonyl group.

More preferred embodiment of X includes chlorine, bromine, and p-toluenesulfonyl group.

Even more preferred embodiment of X includes chlorine.

The compound of formula (1) includes the following (1-A). (1-A)

The compound of formula (1), wherein
R¹ is C₁₋₃ alkyl, and
R^{1A} is C₆₋₁₀ aryl,
or a pharmaceutically acceptable salt thereof, or a solvate thereof.

A preferred embodiment of the compound of formula (1) includes the following (1-B).

### (1-B)

The compound of formula (1), wherein
R¹ is methyl group, and
R^{1A} is phenyl group,
or a pharmaceutically acceptable salt thereof, or a solvate thereof.

The compound of formula (1a) includes the following (1a-A).

(1a-A)

The compound of formula (1a), wherein
R² is C₁₋₃ alkyl,
or a pharmaceutically acceptable salt thereof, or a solvate thereof.

A preferred embodiment of the compound of formula (1a) includes the following (1a-B).

(1a-B)

The compound of formula (1a), wherein
R² is ethyl group,
or a pharmaceutically acceptable salt thereof, or a solvate thereof.

The compound of formula (2) includes the following (2-A). (2-A)

The compound of formula (2), wherein
R¹ and R² are independently C₁₋₃ alkyl, and
R^{1A} is C₆₋₁₀ aryl,
or a pharmaceutically acceptable salt thereof, or a solvate thereof.

A preferred embodiment of the compound of formula (2) includes the following (2-B).

### (2-B)

The compound of formula (2), wherein
R¹ is methyl group,
R² is ethyl group, and
R^{1A} is phenyl group,
or a pharmaceutically acceptable salt thereof, or a solvate thereof.

The compound of formula (2a) includes the following (2a-A).

(2a-A)

The compound of formula (2a), wherein
R^{5a}, R^{5b}, and R^{5c} are independently C₁₋₄ alkyl or phenyl group,
or a pharmaceutically acceptable salt thereof, or a solvate thereof.

A preferred embodiment of the compound of formula (2a) includes the following (2a-B).

(2a-B)

The compound of formula (2a), wherein
R^{5a}, R^{5b}, and R^{5c} are methyl group,
or a pharmaceutically acceptable salt thereof, or a solvate thereof.

The compound of formula (3) includes the following (3-A).

### (3-A)

The compound of formula (3), wherein
R¹ and R² are independently C₁₋₃ alkyl, and
R^{1A} is C₆₋₁₀ aryl,
or a pharmaceutically acceptable salt thereof, or a solvate thereof.

A preferred embodiment of the compound of formula (3) includes the following (3-B).

### (3-B)

The compound of formula (3), wherein
R¹ is methyl group,
R² is ethyl group, and
R^{1A} is phenyl group,
or a pharmaceutically acceptable salt thereof, or a solvate thereof.

The compound of formula (4) includes the following (4-A).

### (4-A)

The compound of formula (4), wherein
R¹ and R² are independently C₁₋₃ alkyl, and
R^{1A} is C₆₋₁₀ aryl,
or a pharmaceutically acceptable salt thereof, or a solvate thereof.

A preferred embodiment of the compound of formula (4) includes the following (4-B).

### (4-B)

The compound of formula (4), wherein
R¹ is methyl group,
R² is ethyl group, and
R^{1A} is phenyl group,
or a pharmaceutically acceptable salt thereof, or a solvate thereof.

The compound of formula (5) includes the following (5-A).

### (5-A)

The compound of formula (5), wherein
R² is C₁₋₃ alkyl,
or a pharmaceutically acceptable salt thereof, or a solvate thereof.

A preferred embodiment of the compound of formula (5) includes the following (5-B).

### (5-B)

The compound of formula (5), wherein
R² is ethyl group,
or a pharmaceutically acceptable salt thereof, or a solvate thereof.

The compound of formula (7) includes the following (7-A).

### (7-A)

The compound of formula (7), wherein
R³ is tert-butoxycarbonyl group,
or a pharmaceutically acceptable salt thereof, or a solvate thereof.

The compound of formula (8) includes the following (8-A).

### (8-A)

The compound of formula (8), wherein
R³ is tert-butoxycarbonyl group,
or a pharmaceutically acceptable salt thereof, or a solvate thereof.

The compound of formula (8a) includes the following (8a-A).

(8a-A)

The compound of formula (8a), wherein
a, b, c, d are independently 1 or 2, and
R⁴ is tert-butoxycarbonyl group,
or a pharmaceutically acceptable salt thereof, or a solvate thereof.

A preferred embodiment of the compound of formula (8a) includes the following (8a-B).

(8a-B)

The compound of formula (8a), wherein
a and c are 1,
b and d are 2, and
R⁴ is tert-butoxycarbonyl group,
or a pharmaceutically acceptable salt thereof, or a solvate thereof.

The compound of formula (9) includes the following (9-A). (9-A)

The compound of formula (9), wherein
a, b, c, d are independently 1 or 2, and
R³ and R⁴ are tert-butoxycarbonyl group,
or a pharmaceutically acceptable salt thereof, or a solvate thereof.

A preferred embodiment of the compound of formula (9) includes the following (9-B).

### (9-B)

The compound of formula (9), wherein
a and c are 1,
b and d are 2, and
R³ and R⁴ are tert-butoxycarbonyl group,
or a pharmaceutically acceptable salt thereof, or a solvate thereof.

The compound of formula (10) includes the following (10-A).

### (10-A)

The compound of formula (10), wherein
a, b, c, d are independently 1 or 2, and
R³ and R⁴ are tert-butoxycarbonyl group,
or a pharmaceutically acceptable salt thereof, or a solvate thereof.

A preferred embodiment of the compound of formula (10) includes the following (10-B).

### (10-B)

The compound of formula (10), wherein
a and c are 1,
b and d are 2, and
R³ and R⁴ are tert-butoxycarbonyl group,
or a pharmaceutically acceptable salt thereof, or a solvate thereof.

The compound of formula (11) includes the following (11-A). (11-A)

The compound of formula (11), wherein
a, b, c, d are independently 1 or 2,
or a pharmaceutically acceptable salt thereof, or a solvate thereof.

A preferred embodiment of the compound of formula (11) includes the following (11-B).

### (11-B)

The compound of formula (11), wherein
a are c are 1, and
b are d are 2,
or a pharmaceutically acceptable salt thereof, or a solvate thereof.

The compound of formula (11a) includes the following (11a-A).

(11a-A)

The compound of formula (11a), wherein
X is chlorine, bromine, or p-toluenesulfonyl group,
or a pharmaceutically acceptable salt thereof, or a solvate thereof.

A preferred embodiment of the compound of formula (11a) includes the following (11a-B).

(11a-B)

The compound of formula (11a), wherein
X is chlorine,
or a pharmaceutically acceptable salt thereof, or a solvate thereof.

The compound of formula (12) includes the following (12-A). (12-A)

The compound of formula (12), wherein
a, b, c, d are independently 1 or 2,
or a pharmaceutically acceptable salt thereof, or a solvate thereof.

A preferred embodiment of the compound of formula (12) includes the following (12-B).

### (12-B)

The compound of formula (12), wherein
a and c are 1, and
b and d are 2,
or a pharmaceutically acceptable salt thereof, or a solvate thereof.

The compound of formula (12a) includes the following (12a-A).

(12a-A)

The compound of formula (12a), wherein
a, b, c, d are independently 1 or 2,
or a pharmaceutically acceptable salt thereof, or a solvate thereof.

A preferred embodiment of the compound of formula (12a) includes the following (12a-B).

(12a-B)

The compound of formula (12a), wherein
a and c are 1, and
b and d are 2,
or a pharmaceutically acceptable salt thereof, or a solvate thereof.

The compound of formula (13) includes the following (13-A). (13-A)

The compound of formula (13), wherein
R¹ is C₁₋₃ alkyl,
R^{1A} is C₆₋₁₀ aryl, and
R^{5a}, R^{5b}, and R^{5c} are independently C₁₋₄ alkyl,
or a pharmaceutically acceptable salt thereof, or a solvate thereof.

A preferred embodiment of the compound of formula (13) includes the following (13-B).

### (13-B)

The compound of formula (13), wherein
R¹ is methyl group,
R^{1A} is phenyl group, and
R^{5a}, R^{5b}, and R^{5c} are methyl group,
or a pharmaceutically acceptable salt thereof, or a solvate thereof.

The compound of formula (14) includes the following (14-A). (14-A)

The compound of formula (14), wherein
R¹ and R² are independently C₁₋₃ alkyl, and
R^{1A} is C₆₋₁₀ aryl,
or a pharmaceutically acceptable salt thereof, or a solvate thereof.

A preferred embodiment of the compound of formula (14) includes the following (14-B).

### (14-B)

The compound of formula (14), wherein
R¹ is methyl group,
R² is ethyl group, and
R^{1A} is phenyl group,
or a pharmaceutically acceptable salt thereof, or a solvate thereof.

The compound of formula (15) includes the following (15-A).

### (15-A)

The compound of formula (15), wherein
R² is C₁₋₃ alkyl,
or a pharmaceutically acceptable salt thereof, or a solvate thereof.

A preferred embodiment of the compound of formula (15) includes the following (15-B).

### (15-B)

The compound of formula (15), wherein
R² is ethyl group,
or a pharmaceutically acceptable salt thereof, or a solvate thereof.

The compound of formula (16) includes the following (16-A).

### (16-A)

The compound of formula (16), wherein
R² is C₁₋₃ alkyl, and
R³ is tert-butoxycarbonyl group,
or a pharmaceutically acceptable salt thereof, or a solvate thereof.

A preferred embodiment of the compound of formula (16) includes the following (16-B).

### (16-B)

The compound of formula (16), wherein
R² is ethyl group, and
R³ is tert-butoxycarbonyl group,
or a pharmaceutically acceptable salt thereof, or a solvate thereof.

Next, the present invention is explained in further detail with preferred embodiments, but the technical scope of the present invention is not limited to these preferred embodiments. The following things may be changed without departing from the scope of the present disclosure. The compound names given in the preferred embodiments below do not necessarily follow the IUPAC nomenclature.

The methods for preparing the present compounds (1), (1a), (2), (2a), (3), (4), (5), (6), (7), (8), (8a), (9), (10), (11), (11a), (12), (12a), (13), (14), (15), and (16), or a tautomer thereof, a stereoisomers thereof, a mixture thereof, or a racemate thereof, or an optionally pharmaceutically acceptable salt or a solvate thereof, which are used as intermediates for the production of the optically active azabicyclo ring derivative (formula (12) are described below. The optically active azabicyclo ring derivative of formula (12) and intermediates thereof can be prepared from known compounds by the following production methods or methods analogous thereto, or by synthesis known to those skilled in the art. It can be produced by appropriately combining methods.

In addition, the compound obtained in each step can be used in the next reaction as a reaction solution or as a reaction composition, but it can also be isolated from the reaction mixture according to a conventional method, and can be easily purified by separation means such as recrystallization, distillation, and chromatography.

Each symbol of the compounds in the following reactions has the same meaning as described above unless otherwise indicated.

The manufacturing methods of the present invention are described below. Starting materials which are not described below are commercially available or can be produced by methods known to those skilled in the art or methods based thereon.

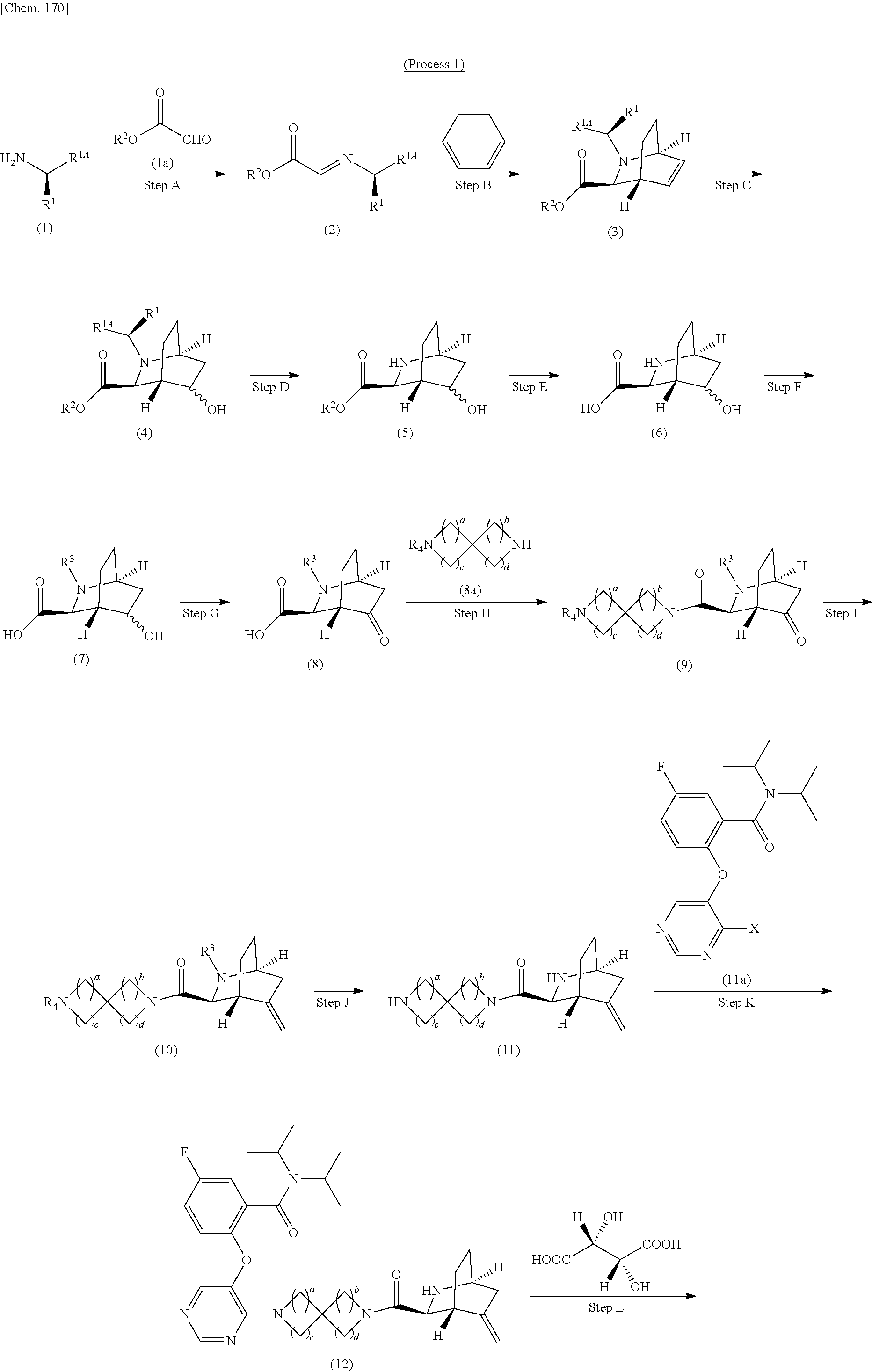

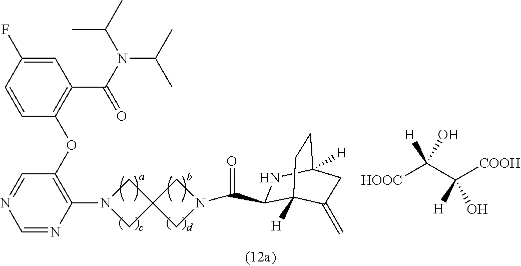

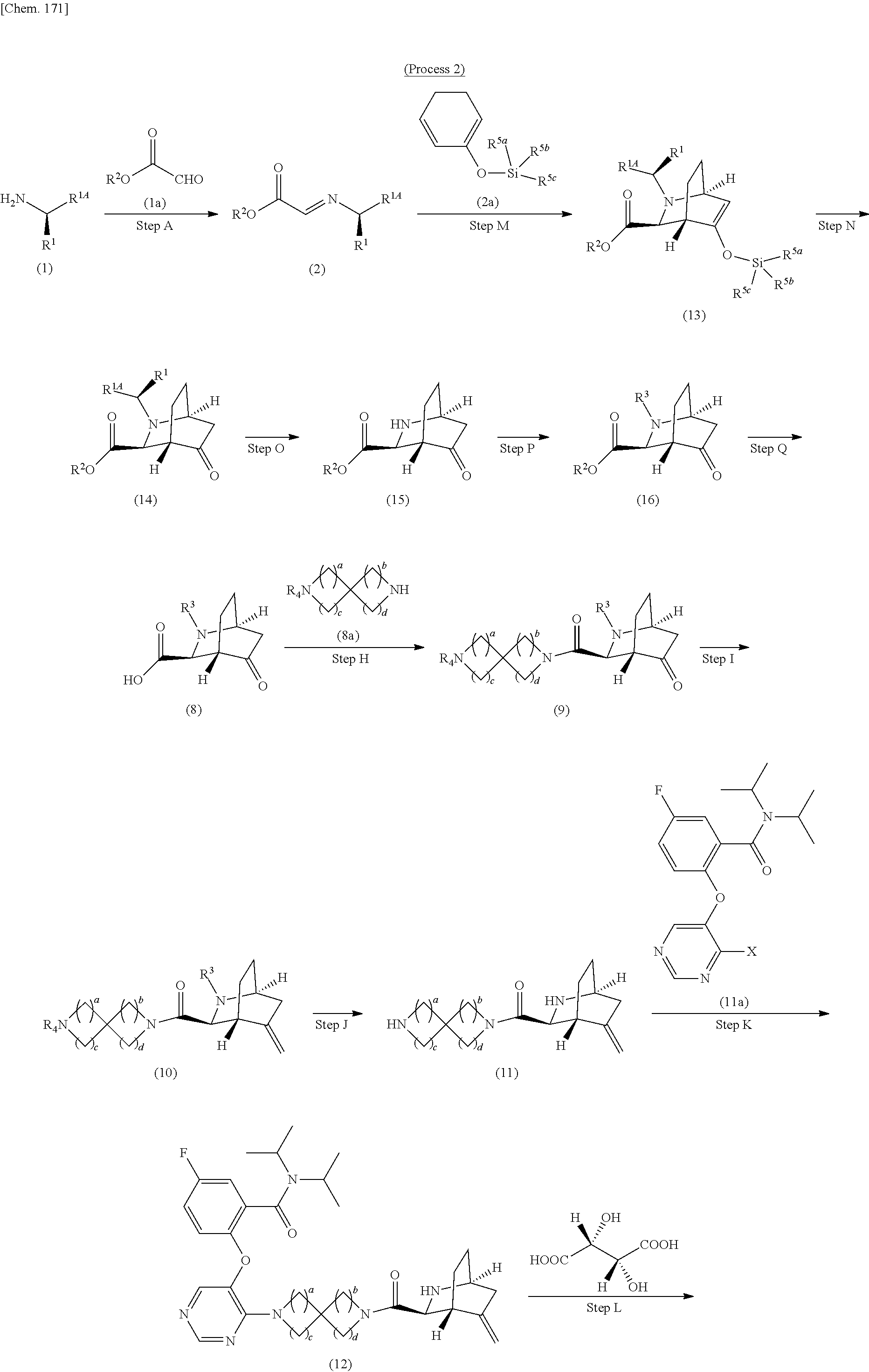

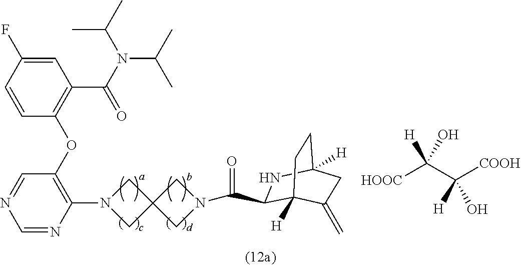

wherein R¹, R^{1A}, R², R³, R⁴, R^{5a}, R^{5b}, R^{5c}, a, b, c, and d are as defined in Item 1 and/or other Items.

Steps A, B, C, D, E, F, G, H, K, I, L, M, N, O, P, and Q are explained below with preferred embodiments, but the present disclosure is limited thereto.

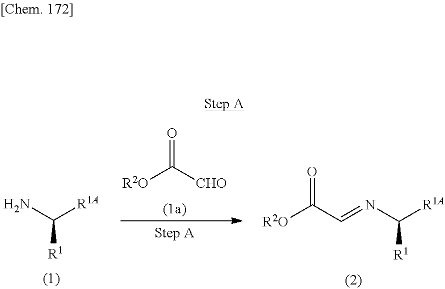

wherein R¹, R^{1A}, and R² are as defined in Item 1 and/or other Items.

The present step is a step of obtaining the compound of formula (2) by reacting the compound of formula (1) with the compound of formula (1a) in a solvent.

The solvent used herein is not particularly limited as long as its boiling point is higher than the reaction temperature of the present reaction, which includes a halogen solvent, preferably dichloromethane, chloroform, 1,2-dichloroethane, and chlorobenzene, more preferably dichloromethane.

The amount of the solvent used herein is generally 2 parts by weight to 20 parts by weight based on the weight of the compound of formula (1), preferably 5 parts by weight to 20 parts by weight, more preferably 8 parts by weight to 15 parts by weight.

The amount of the compound of formula (1a) to be used is generally 1.0 eq-10.0 eq relative to 1 eq of the compound of formula (1), preferably 1.0 eq-5.0 eq, more preferably 1.0 eq-2.0 eq, the most preferably 1.0 eq-1.2 eq.

The reaction time is generally about 0.5 hours to 12 hours, preferably 0.5 hours to 8 hours.

The reaction temperature is generally −30 °C to 120 °C, preferably −30 °C to 130 °C, more preferably −30 °C to 50 °C, even more preferably −20 °C to 30 °C.

As an additive for accelerating the reaction, a desiccant agent may be added, and the amount thereof is preferably 1.0 part by weight to 5.0 parts by weight based on the weight of the compound of formula (1a). The desiccant agent includes sodium sulfate, magnesium sulfate, and molecular sieves, more preferably sodium sulfate.

R¹ and R² are preferably as defined above.

Instead of the compound of formula (1a), the compounds of formula (1a-B) and formula (1a-C) obtained by the following method can be used.

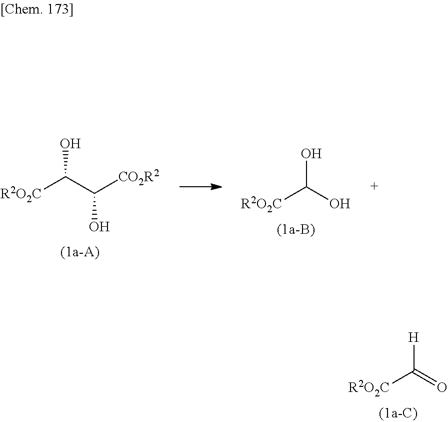

wherein R² is as defined in Item 1 and/or other Items.

The present step is a step of obtaining the compounds of formulae (1a-B) and (1a-C) by oxidizing the compound of formula (1a-A) in a solvent.

The solvent used herein is not particularly limited as long as its boiling point is higher than the reaction temperature of the present reaction, which includes a halogen solvent and a mixture of a halogen solvent and water, preferably dichloromethane, chloroform, 1,2-dichloroethane, chlorobenzene, and a mixture any halogen solvent and water, more preferably dichloromethane and a mixture of dichloromethane and water.

The oxidation agent used herein includes 2,2,6,6-tetramethylpiperidine 1-oxyl, 1-methyl-2-azaadamantane-N-oxyl, 2-hydroxy-2-azaadamantane, 9-azanoradamantan-N-oxyl, 1,1,1-triacetoxy-1,1-dihydro-1,2-benziodoxol-3-(1H)-one, potassium 2-iodo-5-methylbenzenesulfonate, “a combination of DMSO and oxalyl chloride”, acetic anhydride, sulfur trioxide-pyridine-complex, N,N′-dicyclohexylcarbodiimide, chromium trioxide, tetrapropylammonium perruthenate, sodium hypochlorite pentahydrate, and sodium periodate, preferably sodium periodate.

The amount of the solvent used herein is generally 2 parts by weight to 20 parts by weight based on the weight of the compound of formula (1a-A), preferably 5 parts by weight to 20 parts by weight, more preferably 8 parts by weight to 15 parts by weight.

The amount of the oxidation agent used herein is generally 1.0 eq-5.0 eq relative to 1 eq of the compound of formula (1a-A), preferably 1.0 eq-2.0 eq, more preferably 1.0 eq-1.5 eq.

The reaction time is generally about 1 hour to 48 hours, preferably 5 hours to 36 hours, more preferably 12 hours to 24 hours.

The reaction temperature is generally 0 °C to 50 °C, preferably 5 °C to 40 °C, more preferably 10 °C to 30 °C.

R² is preferably as defined above.

### Step B

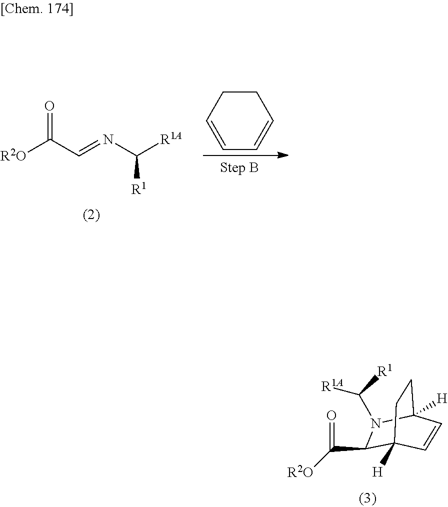

wherein R¹, R^{1A}, and R² are as defined in Item 1 and/or other Items.

The present step is a step of obtaining the compound of formula (3) by reacting the compound of formula (2) with 1,3-cyclohexadiene in the presence of a solvent and an acid.

The solvent used herein is not particularly limited as long as its boiling point is higher than the reaction temperature of the present reaction, which includes a halogen solvent, preferably dichloromethane, chloroform, 1,2-dichloroethane, and chlorobenzene, and more preferably dichloromethane.

The acid used herein includes an organic acid, preferably trifluoroacetic acid, boron trifluoride-ethyl ether complex, and a mixture thereof, and more preferably a mixture of trifluoroacetic acid and boron trifluoride-ethyl ether complex.

The amount of the solvent used herein is generally 2 parts by weight to 20 parts by weight based on the weight of the compound of formula (2), preferably 3 parts by weight to 10 parts by weight, more preferably 4 parts by weight to 8 parts by weight.

The amount of 1,3-cyclohexadiene used herein is generally 1.0 eq-10.0 eq relative to 1 eq of the compound of formula (2), preferably 1.0 eq-5.0 eq, more preferably 1.0 eq-2.0 eq, the most preferably 1.0 eq-1.6 eq.

The amount of the acid used herein is generally 0.3 eq-3.0 eq relative to 1 eq of the compound of formula (2), preferably 0.5 eq-2.0 eq, more preferably 0.5 eq-1.5 eq, the most preferably 0.8 eq-1.2 eq.

The reaction time is generally about 0.5 hours to 24 hours, preferably 5 hours to 20 hours.

The reaction temperature is generally −80 °C to 60 °C, preferably −70 °C to 50 °C, more preferably −70 °C to 30 °C.

R¹ and R² are preferably as defined above.

### Step C

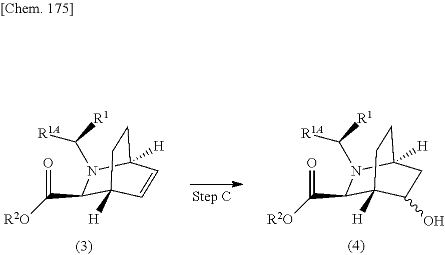

wherein R¹, R^{1A}, and R² are as defined in Item 1 and/or other Items.

The present step is a step of obtaining the compound of formula (4) by the compound of formula (3) with borane in the presence of a solvent, followed by reacting with a peroxide and a base.

The solvent used herein is not particularly limited as long as its boiling point is higher than the reaction temperature of the present reaction, which includes ether solvent, preferably diethyl ether, tetrahydrofuran, and methyl tert-butyl ether, and more preferably methyl tert-butyl ether.

The boranes used herein includes borane and 9-borabicyclo[3.3.1]nonane, which includes a dimerized one and a complex thereof with a solvent, preferably borane-tetrahydrofuran complex.

The base used herein includes inorganic bases, preferably lithium hydroxide, sodium hydroxide, and potassium hydroxide, more preferably sodium hydroxide.

The peroxide used herein includes hydrogen peroxide, cumene hydroperoxide, benzoyl peroxide, peracetic acid and sodium peroxoborate, which includes hydrate thereof, preferably hydrogen peroxide and sodium peroxoborate, which includes hydrate thereof, more preferably sodium peroxoborate, which includes hydrate thereof.

The amount of the solvent used herein is generally 1 parts by weight to 20 parts by weight based on the weight of the compound of formula (3), preferably 1 parts by weight to 10 parts by weight, more preferably 1 parts by weight to 2 parts by weight.

The amount of borane used herein is generally 1.0 eq-10.0 eq relative to 1 eq of the compound of formula (3), preferably 1.0 eq-5.0 eq, more preferably 1.0 eq-2.0 eq, the most preferably 1.0 eq-1.6 eq.

The amount of peroxide used herein is generally 1.0 eq-20.0 eq relative to 1 eq of the compound of formula (3), preferably 1.0 eq-10.0 eq, more preferably 1.0 eq-6.0 eq.

The reaction time is generally about 0.5 hours to 24 hours, preferably 3 hours to 20 hours.

The reaction temperature is generally −50 °C to 50 °C, preferably −40 °C to 40 °C, more preferably −30 °C to 30 °C.

R¹ and R² are preferably as defined above.

### Step D

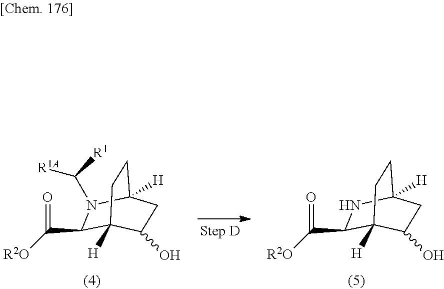

wherein R¹, R^{1A}, and R² are as defined in Item 1 and/or other Items.

The present step is a step of obtaining the compound of formula (5) by reacting the compound of formula (4) with hydrogen in the presence of a solvent and a catalyst.

The solvent used herein is not particularly limited as long as its boiling point is higher than the reaction temperature of the present reaction, which includes alcohol solvent, preferably ethanol and methanol, and more preferably methanol.

The catalyst used herein includes preferably Pd/C.

The amount of the solvent used herein is generally 2 parts by weight to 20 parts by weight based on the weight of the compound of formula (4), preferably 2 parts by weight to 10 parts by weight, more preferably 2 parts by weight to 5 parts by weight.

The amount of the catalyst used herein is generally 1.0 parts by weight to 30.0 parts by weight based on the weight of the compound of formula (4), preferably 5.0 parts by weight to 20.0 parts by weight, more preferably 7.0 parts by weight to 15.0 parts by weight.

The reaction time is generally about 0.5 hours to 48 hours, preferably 12 hours to 30 hours.

The reaction temperature is generally 0 °C to 60 °C, preferably 10 °C to 50 °C, more preferably 15 °C to 40 °C.

R¹ and R² are preferably as defined above.

### Step E

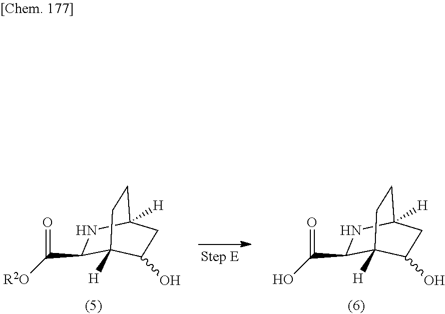

wherein R² is as defined in Item 1 and/or other Items.

The present step is a step of obtaining the compound of formula (6) by reacting the compound of formula (5) with a base in the presence of a solvent.

The solvent used herein is not particularly limited as long as its boiling point is higher than the reaction temperature of the present reaction, which includes ether solvent, preferably tetrahydrofuran and 1,4-dioxane, and more preferably dioxane.

The base used herein includes preferably sodium hydroxide and potassium hydroxide, more preferably sodium hydroxide. The base may be used as an aqueous solution thereof.

The amount of the solvent used herein is generally 2 parts by weight to 20 parts by weight based on the weight of the compound of formula (5), preferably 5 parts by weight to 15 parts by weight, more preferably 8 parts by weight to 15 parts by weight.

The amount of the base used herein is generally 1.0 eq-3.0 eq relative to 1 eq of the compound of formula (5), preferably 1.0 eq-2.0 eq, more preferably 1.0 eq-1.5 eq.

The reaction time is generally about 0.5 hours to 24 hours, preferably 1 hour to 10 hours, more preferably 2 hours to 5 hours.

The reaction temperature is generally 0 °C to 60 °C, preferably 0 °C to 40 °C, more preferably 5 °C to 30 °C.

R² is preferably as defined above.

### Step F

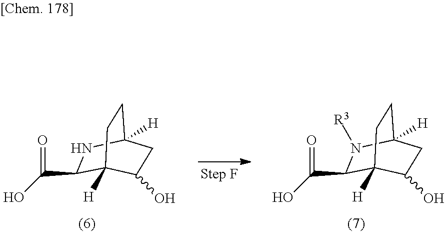

wherein R³ is as defined in Item 1 and/or other Items.

The present step is a step of obtaining the compound of formula (7) by reacting the compound of (6) with a reagent for protecting amino group in the presence of a solvent.

The solvent used herein is not particularly limited as long as its boiling point is higher than the reaction temperature of the present reaction, which includes ether solvent, preferably tetrahydrofuran and 1,4-dioxane, and more preferably dioxane.

The reagent for protecting amino group used herein includes preferably di-tert-butyl dicarbonate, benzyloxycarbonyl chloride, more preferably di-tert-butyl dicarbonate.

The amount of the solvent used herein is generally 2 parts by weight to 20 parts by weight based on the weight of the compound of formula (6), preferably 5 parts by weight to 15 parts by weight, more preferably 8 parts by weight to 15 parts by weight.

The amount of the reagent for protecting amino group used herein is generally 1.0 eq-3.0 eq relative to 1 eq of the compound of formula (6), preferably 1.0 eq-2.0 eq, more preferably 1.0 eq-1.5 eq.

The reaction time is generally about 0.5 hours to 48 hours, preferably 5 hours to 36 hours, more preferably 12 hours to 24 hours.

The reaction temperature is generally −20 °C to 50 °C, preferably −10 °C to 40 °C, more preferably 0 °C to 30 °C.

R³ is preferably as defined above.

### Step G

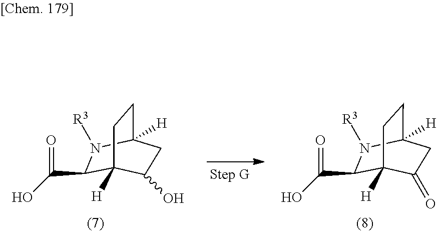

wherein R³ is as defined in Item 1 and/or other Items.

The present step is a step of obtaining the compound of formula (8) by reacting the compound of formula (7) with an oxidation agent and a re-oxidation agent in the presence of a solvent.

The solvent used herein is not particularly limited as long as its boiling point is higher than the reaction temperature of the present reaction, which includes an ester solvent and a halogen solvent, preferably an ester solvent, and more preferably ethyl acetate.

The oxidation agent used herein includes 2,2,6,6-tetramethylpiperidine 1-oxyl, 1-methyl-2-azaadamantane-N-oxyl, 2-hydroxy-2-azaadamantane, 9-azanoradamantan-N-oxyl, 1,1,1-triacetoxy-1,1-dihydro-1,2-benziodoxol-3-(1H)-one, potassium 2-iodo-5-methylbenzenesulfonate, “a combination of DMSO and oxalyl chloride”, acetic anhydride, sulfur trioxide-pyridine-complex, N,N′-dicyclohexylcarbodiimide, chromium trioxide, tetrapropylammonium perruthenate, and sodium hypochlorite pentahydrate, preferably 2,2,6,6-tetramethylpiperidine 1-oxyl.

The re-oxidation agent used herein includes preferably trichloroisocyanuric acid, sodium hypochlorite, iodobenzene diacetate, air, oxygen, oxone, and morpholine N-oxide, more preferably trichloroisocyanuric acid.

The amount of the solvent used herein is generally 2 parts by weight to 80 parts by weight based on the weight of the compound of formula (7), preferably 10 parts by weight to 70 parts by weight, more preferably 20 parts by weight to 60 parts by weight.

The amount of the oxidation agent used herein is generally 0.01 eq-0.2 eq relative to 1 eq of the compound of formula (7), preferably 0.01 eq-0.1 eq, more preferably 0.01 eq-0.08 eq.

The amount of the re-oxidation agent used herein is generally 0.3 eq-2.0 eq relative to 1 eq of the compound of formula (7), preferably 0.3 eq-1.5 eq, more preferably 0.3 eq-1.0 eq.

The reaction time is generally about 0.5 hours to 15 hours, preferably 1 hour to 12 hours, more preferably 1 hour to 8 hours.

The reaction temperature is generally −20 °C to 50 °C, preferably −10 °C to 30 °C, more preferably −10 °C to 10 °C.

R³ is preferably as defined above.

### Step H

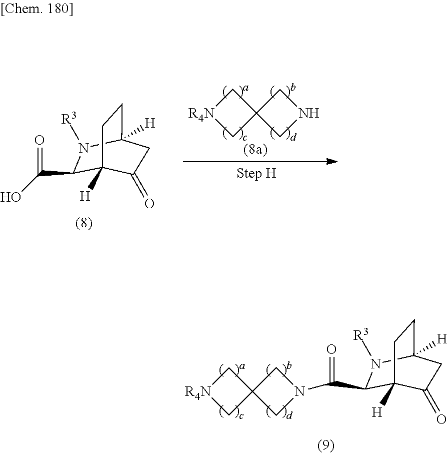

wherein a, b, c, d, R³ and R⁴ are as defined in Item 1 and/or other Items.

The present step is a step of obtaining the compound of formula (9) by reacting the compound of formula (8) with the compound of formula (8a) in the presence of a solvent, a base and a condensation agent.

The solvent used herein is not particularly limited as long as its boiling point is higher than the reaction temperature of the present reaction, which includes an amide solvent, preferably N,N-dimethylformamide and N-methyl-2-pyrrolidone, and more preferably N,N-dimethylformamide.

The base used herein includes organic bases, preferably triethylamine, diisopropylethylamine, and pyridine, more preferably triethylamine and N,N-diisopropylethylamine, the most preferably triethylamine.

The condensation agent used herein includes thionyl chloride, diphenylphosphoryl azide, propylphosphonic anhydride, 1,1-carbonyldiimidazole, 1H-benzotriazol-1-yloxytripyrrolidinophosphonium hexafluorophosphate, {{[(1-cyano-2-ethoxy-2-oxoethylidene)amino]oxy}-4-morpholinomethylene}dimethylammonium hexafluorophosphate, O-(7-azabenzotriazol-1-yl)-N,N,N′N′-tetramethyluronium hexafluorophosphate, 1-hydroxyazabenzotriazole, 1-hydroxybenzotriazole, 1-ethyl-3-(3-dimethylaminopropyl)carbodiimide hydrochloride, N,N′-dicyclohexylcarbodiimide, N,N′-diisopropylcarbodiimide, and a mixture thereof, preferably a mixture of 1-hydroxybenzotriazole and 1-ethyl-3-(3-dimethylaminopropyl)carbodiimide hydrochloride.

The amount of the solvent used herein is generally 2 parts by weight to 20 parts by weight based on the weight of the compound of formula (8), preferably 5 parts by weight to 15 parts by weight, more preferably 7 parts by weight to 12 parts by weight.

The amount of the compound of formula (8a) to be used is generally 1.0 eq-10.0 eq relative to 1 eq of the compound of formula (8), preferably 1.0 eq-5.0 eq, more preferably 1.0 eq-2.0 eq, the most preferably 1.0 eq-1.5 eq.

The amount of the base used herein is generally 1.0 eq-10.0 eq relative to 1 eq of the compound of formula (8), preferably 1.0 eq-5.0 eq, more preferably 1.0 eq-2.0 eq, the most preferably 1.0 eq-1.6 eq.

The amount of the condensation agent used herein is generally 1.0 eq-10.0 eq relative to 1 eq of the compound of formula (8), preferably 1.0 eq-5.0 eq, more preferably 1.0 eq-2.0 eq, the most preferably 1.0 eq-1.6 eq.

The reaction time is generally about 0.5 hours to 24 hours, preferably 0.5 hours to 5 hours, more preferably 0.5 hours to 2 hours.

The reaction temperature is generally 0 °C to 60 °C, preferably 0 °C to 40 °C, more preferably 10 °C to 30 °C.

R³ and R⁴ are preferably as defined above.

### Step I

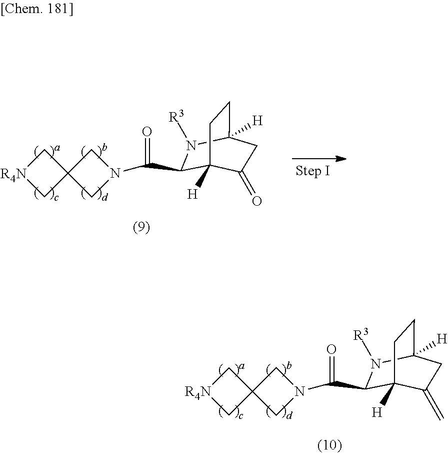

wherein a, b, c, d, R³ and R⁴ are as defined in Item 1 and/or other Items.

The present step is a step of obtaining the compound of formula (10) by reacting the compound of formula (9) with halogenated methyltriphenylphosphonium in the presence of a solvent and a base.

The solvent used herein is not particularly limited as long as its boiling point is higher than the reaction temperature of the present reaction, which includes an ether solvent, preferably diethyl ether, tetrahydrofuran, and methyl tert-butyl ether, and more preferably tetrahydrofuran.

The base used herein includes inorganic bases, preferably sodium tert-butoxide, potassium tert-butoxide, and sodium methoxide, more preferably sodium tert-butoxide and potassium tert-butoxide.

The halogenated methyltriphenylphosphonium used herein includes methyltriphenylphosphonium bromide and methyltriphenylphosphonium iodide, preferably methyltriphenylphosphonium bromide.

The amount of the solvent used herein is generally 2 parts by weight to 30 parts by weight based on the weight of the compound of formula (9), preferably 5 parts by weight to 20 parts by weight, more preferably 8 parts by weight to 15 parts by weight.

The amount of the halogenated methyltriphenylphosphonium used herein is generally 1.0 eq-10.0 eq relative to 1 eq of the compound of formula (9), preferably 1.0 eq-5.0 eq. more preferably 1.0 eq-3.0 eq, the most preferably 1.5 eq-2.5 eq.

The amount of the base used herein is generally 1.0 eq-10.0 eq relative to 1 eq of the compound of formula (9), preferably 1.0 eq-5.0 eq, more preferably 1.0 eq-3.0 eq, the most preferably 1.5 eq-2.0 eq.

The reaction time is generally about 0.5 hours to 24 hours, preferably 0.5 hours to 6 hours, more preferably 1 hour to 3 hours.

The reaction temperature is generally 0 °C to 100 °C, preferably 10 °C to 60 °C, more preferably 10 °C to 40 °C.

R³ and R⁴ are preferably as defined above.

### Step J

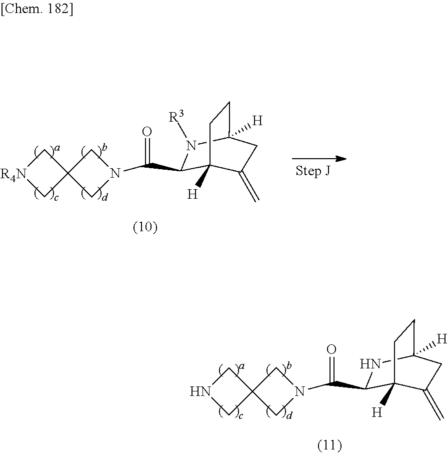

wherein a, b, c, d, R³, and R⁴ are as defined in Item 1 and/or other Items.

The present step is a step of obtaining the compound of formula (11) by reacting the compound of formula (10) with an acid in the presence of a solvent.

The solvent used herein is not particularly limited as long as its boiling point is higher than the reaction temperature of the present reaction, which includes an ether solvent, preferably diethyl ether, tetrahydrofuran, and methyl tert-butyl ether, more preferably tetrahydrofuran.

The acid used herein includes preferably hydrochloric acid, hydrobromic acid, and trifluoroacetic acid, more preferably hydrochloric acid.

The amount of the acid used herein is generally 1.0 eq-20.0 eq relative to 1 eq of the compound of formula (10), preferably 5.0 eq-15.0 eq, more preferably 7.0 eq-12.0 eq.

The reaction time is generally about 0.5 hours to 24 hours, preferably 0.5 hours to 6 hours, more preferably 1 hour to 5 hours.

The reaction temperature is generally 0 °C to 100 °C, preferably 10 °C to 80 °C, more preferably 20 °C to 50 °C.

### Step K

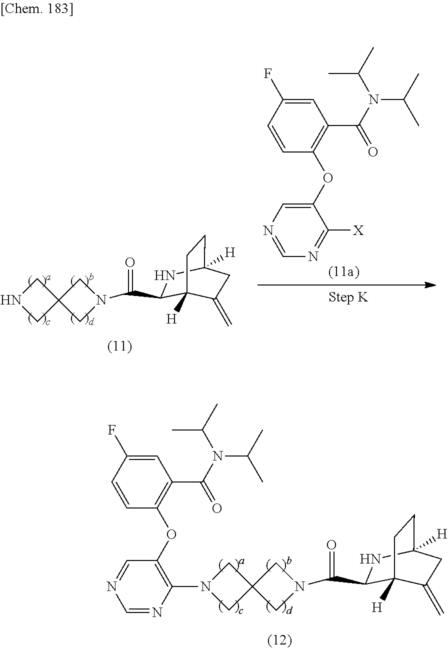

wherein a, b, c, d, and X are as defined in Item 1 and/or other Items.

The present step is a step of obtaining the compound of formula (12) by reacting the compound of formula (11) with the compound of formula (11a) in the presence of a solvent and a base.

The solvent used herein is not particularly limited as long as its boiling point is higher than the reaction temperature of the present reaction, which includes an ether solvent and acetonitrile, and preferably acetonitrile.

The base used herein includes organic bases, preferably triethylamine, diisopropylethylamine, and pyridine, more preferably triethylamine and N,N-diisopropylethylamine, the most preferably triethylamine.

The amount of the solvent used herein is generally 2 parts by weight to 20 parts by weight based on the weight of the compound of formula (11), preferably 2 parts by weight to 10 parts by weight, more preferably 2 parts by weight to 6 parts by weight.

The amount of the base used herein is generally 1.0 eq-30.0 eq relative to 1 eq of the compound of formula (11), preferably 5.0 eq-25.0 eq, more preferably 10.0 eq-20.0 eq.

The amount of the compound of formula (11a) to be used is generally 0.5 eq-3.0 eq relative to 1 eq of the compound of formula (11), preferably 0.6 eq-1.5 eq, more preferably 0.6 eq-1.2 eq.

The reaction time is generally about 0.5 hours to 48 hours, preferably 5 hours to 48 hours, more preferably 10 hours to 48 hours.

The reaction temperature is generally −20 °C to 60 °C, preferably −10 °C to 30 °C, more preferably −5 °C to 20 °C.

### Step L

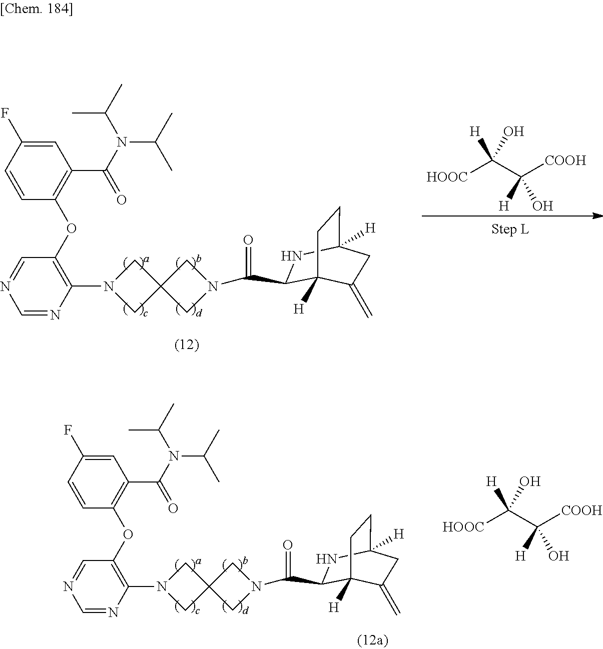

wherein a, b, c, and d are as defined in Item 1 and/or other Items.

The present step is a step of obtaining the compound of formula (12a) by reacting the compound of formula (12) with L(+)-tartaric acid in the presence of a solvent.

The solvent used herein is not particularly limited as long as its boiling point is higher than the reaction temperature of the present reaction, which includes an alcohol solvent and a mixture of an alcohol solvent and water, preferably methanol, ethanol, 2-propyl alcohol, and a mixture of any alcohol and water, and more preferably ethanol and a mixture of ethanol and water.

The amount of the alcohol solvent used herein is generally 2 parts by weight to 30 parts by weight based on the weight of the compound of formula (12), preferably 5 parts by weight to 20 parts by weight, more preferably 8 parts by weight to 15 parts by weight.

The amount of water used herein is generally 0.01 parts by weight to 2 parts by weight based on the weight of the compound of formula (12), preferably 0.05 parts by weight to 1.5 parts by weight, more preferably 0.05 parts by weight to 1 part by weight.

The amount of L(+)-tartaric acid used herein is generally 0.5 eq-2.0 eq relative to 1 eq of the compound of formula (12), preferably 0.7 eq-1.5 eq, more preferably 1.0 eq-1.5 eq.

The reaction time is generally about 0.5 hours to 5 hours, preferably 0.5 hours to 3 hours, more preferably 0.5 hours to 2 hours.

The reaction temperature is generally 0 °C to 80 °C, preferably 20 °C to 70 °C, more preferably 40 °C to 70 °C.

### Step M

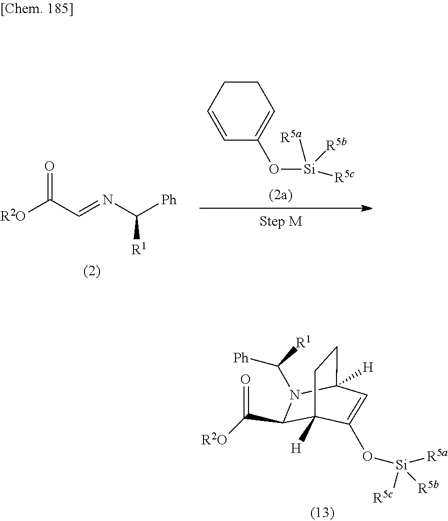

wherein R¹, R², R^{5a}, R^{5b}, and R^{5c} are as defined in Item 1 and/or other Items.

The present step is a step of obtaining the compound of formula (13) by reacting the compound of formula (2) with the compound of formula (2a) in the presence of a solvent and an acid.

The solvent used herein is not particularly limited as long as its boiling point is higher than the reaction temperature of the present reaction, which includes a halogen solvent, preferably dichloromethane, chloroform, 1,2-dichloroethane, and chlorobenzene, and more preferably dichloromethane.

The acid used herein includes organic acids, preferably trifluoroacetic acid, boron trifluoride-ethyl ether complex, and a mixture thereof, more preferably a mixture of trifluoroacetic acid and boron trifluoride-ethyl ether complex.

The amount of the solvent used herein is generally 2 parts by weight to 20 parts by weight based on the weight of the compound of formula (2), preferably 3 parts by weight to 10 parts by weight, more preferably 3 parts by weight to 8 parts by weight.

The amount of the compound of formula (2a) to be used is generally 1.0 eq-10.0 eq relative to 1 eq of the compound of formula (2), preferably 1.0 eq-5.0 eq, more preferably 1.0 eq-2.0 eq, the most preferably 1.0 eq-1.5 eq.

The amount of the acid used herein is generally 1.0 eq-10.0 eq relative to 1 eq of the compound of formula (2), preferably 1.0 eq-5.0 eq, more preferably 1.0 eq-2.0 eq, the most preferably 1.0 eq-1.5 eq.

The reaction time is generally about 0.5 hours to 24 hours, preferably 0.5 hours to 10 hours, more preferably 0.5 hours to 5 hours.

The reaction temperature is generally −100 °C to 30 °C, preferably −90 °C to 20 °C, more preferably −80 °C to 10 °C.

R¹, R², R^{5a}, R^{5b}, and R^{5c} are preferably as defined above.

### Step N

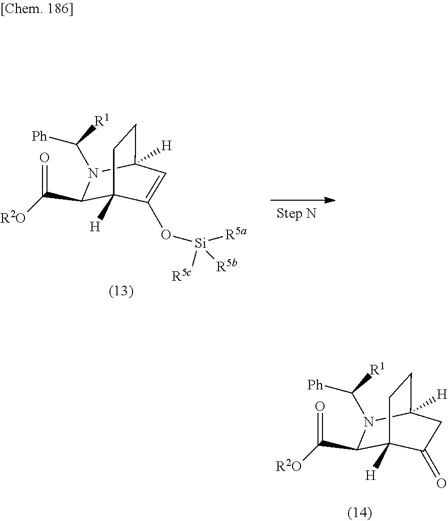

wherein R¹, R², R^{5a}, R^{5b}, and R^{5c} are as defined in Item 1 and/or other Items.

The present step is a step of obtaining the compound of formula (14) by hydrolyzing the compound of formula (13) with water.

The solvent used herein is not particularly limited as long as its boiling point is higher than the reaction temperature of the present reaction, which includes a halogen solvent, preferably dichloromethane, chloroform, 1,2-dichloroethane, and chlorobenzene, and more preferably dichloromethane.

The amount of the solvent used herein is generally 2 parts by weight to 20 parts by weight based on the weight of the compound of formula (13), preferably 3 parts by weight to 10 parts by weight, more preferably 3 parts by weight to 8 parts by weight.

The amount of water used herein is generally 1.0 eq-100.0 eq relative to 1 eq of the compound of formula (13), preferably 5.0 eq-50.0 eq, more preferably 5.0 eq-30.0 eq.

The reaction time is generally about 0.5 hours to 5 hours, preferably 0.5 hours to 3 hours, more preferably 0.5 hours to 2 hours.

The reaction temperature is generally −50 °C to 50 °C, preferably −20 °C to 20 °C, more preferably −10 °C to 10 °C.

R¹, R², R^{5a}, R^{5b}, and R^{5c} are preferably as defined above.

### Steps O and P

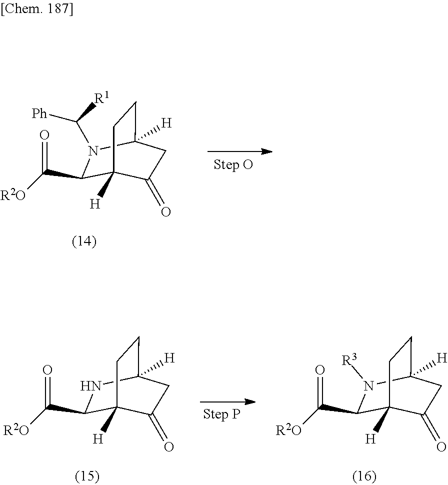

wherein R¹, R² and R³ are as defined in Item 1 and/or other Items.

The present step is a step of obtaining the compound of formula (16) via the compound of formula (15) by reacting the compound of formula (14) with hydrogen in the presence of a solvent, a catalyst and a reagent for protecting amino group.

The solvent used herein is not particularly limited as long as its boiling point is higher than the reaction temperature of the present reaction, which includes an alcohol solvent, preferably ethanol and methanol, and more preferably ethanol.

The catalyst used herein includes preferably Pd/C.

The reagent for protecting amino group used herein includes preferably di-tert-butyl dicarbonate.

The amount of the solvent used herein is generally 0.01 parts by weight to 2.0 parts by weight based on the weight of the compound of formula (14), preferably 0.01 parts by weight to 1.0 parts by weight, more preferably 0.01 parts by weight to 0.5 parts by weight.

The amount of the catalyst used herein is generally 1.0 parts by weight to 30.0 parts by weight based on the weight of the compound of formula (14), preferably 5.0 parts by weight to 20.0 parts by weight, more preferably 7.0 parts by weight to 15.0 parts by weight.

The amount of the reagent for protecting amino group used herein is generally 1.0 eq-3.0 eq relative to 1 eq of the compound of formula (14), preferably 1.0 eq-2.0 eq, more preferably 1.0 eq-1.5 eq.

The reaction time is generally about 0.5 hours to 12 hours, preferably 1 hour to 8 hours, more preferably 2 hours to 6 hours.

The reaction temperature is generally 0 °C to 60 °C, preferably 10 °C to 50 °C, more preferably 15 °C to 40 °C.

R¹, R², and R³ are preferably as defined above.

### Step Q

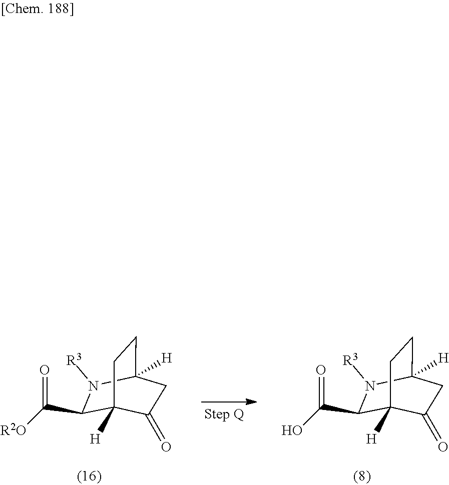

wherein R² and R³ are as defined in Item 1 and/or other Items.

The present step is a step of obtaining the compound of formula (8) by reacting the compound of formula (16) with a base in the presence of a solvent.

The solvent used herein is not particularly limited as long as its boiling point is higher than the reaction temperature of the present reaction, which includes an alcohol solvent, preferably ethanol and methanol, more preferably methanol.

The base used herein includes preferably sodium hydroxide and potassium hydroxide, more preferably sodium hydroxide. The base may be used as an aqueous solution thereof.

The amount of the solvent used herein is generally 2 parts by weight to 50 parts by weight based on the weight of the compound of formula (16), preferably 5 parts by weight to 40 parts by weight, more preferably 8 parts by weight to 35 parts by weight.

The amount of the base used herein is generally 1.0 eq-10.0 eq relative to 1 eq of the compound of formula (16), preferably 2.0 eq-8.0 eq, more preferably 3.0 eq-6.0 eq.

The reaction time is generally about 0.5 hours to 24 hours, preferably 1 hour to 10 hours, more preferably 3 hours to 8 hours.

The reaction temperature is generally 0 °C to 80 °C, preferably 20 °C to 70 °C, more preferably 30 °C to 60 °C.

R² and R³ are preferably as defined above.

The order in which the reagents and the like are added is not limited to those listed above.

The present invention is illustrated in more detail with reference to Reference examples and Examples below, but should not be limited thereto. The compounds were identified by elemental analysis values, mass spectrum, high performance liquid chromatography mass spectrometer (LCMS), infrared absorption (IR) spectrum, nuclear magnetic resonance (NMR) spectrum, high performance liquid chromatography (HPLC), etc.

Each RT is shown in the table below, which was obtained by analyzing each compound under each HPLC measurement condition shown in the table.

Method 1

| | |
|---|---|
| Column | Waters, Xselect CSH C18 3.5 μm, 4.6 × 150 mm |
| Mobile phase A | Purified water with 0.1% HClO₄ |
| Mobile phase B | Acetonitrile |
| Detection | UV = 210 nm |
| wavelength | |
| Temperature of | 40 °C |
| column oven | |
| Flow rate | 1.0 mL/min. |
| Injection volume | 10 μL |
| Ratio of mobile | Time (min) 0.0 1.0 11.0 15.0 15.1 20.0 |
| phases | B. Conc (%) 10 10 100 100 10 Stop |
| Gradient | |
| condition | |
| Analysis Time | 20 min. |
| Retention Time | RT = 6.7 min (compound 8), |
| | RT = 9.0 min (compound 9) |

Method 2

| | |
|---|---|
| Column | Waters, Xselect CSH C18 3.5 μm, 4.6 × 150 mm |
| Mobile phase A | Purified water with 0.01% HClO₄ |
| Mobile phase B | Methanol |
| Detection | UV = 220 mm |
| wavelength | |
| Temperature of | 40 °C |
| column oven | |
| Flow rate | 1.6 mL/min. |
| Injection volume | 5 μL |
| Ratio of mobile | Time (min) 0.0 0.5 12.5 17.5 20.0 20.1 25.0 |
| phases | B. Conc (%) 5 5 50 90 90 5 Stop |
| Gradient | |
| condition | |
| Analysis Time | 25 min. |
| Retention Time | RT = 0.8 min (compound 11), |
| | RT = 16.9 min (compound 9), |
| | RT = 18.1 min (compound 10) |

Method 3

| | |
|---|---|
| Column | Waters, Xselect CSH C18 3.5 μm, 4.6 × 150 mm |
| Mobile phase A | Purified water with 0.01% HClO₄ |
| Mobile phase B | Methanol |
| Detection | UV = 220 nm |
| wavelength | |
| Temperature of | 40 °C |
| column oven | |
| Flow rate | 0.8 mL/min. |
| Injection volume | 5 μL |
| Ratio of mobile | Time (min) 0.0 25.0 35.0 40.0 40.1 50.0 |
| phases | B. Conc (%) 20 50 90 90 20 Stop |
| Gradient | |
| condition | |
| Analysis Time | 50 min. |
| Retention Time | RT = 19.9 min (compound 12) |

Method 4

| | |
|---|---|
| Column | DAICEL, CHIRALPAK ZWIX (+) 3 μm, |
| | 4.0 × 250 mm |
| Mobile phase | MeCN/MeOH/H2O/Et2NH/HCO2H = |
| | 9500/500/50/26/19) |
| Detection | UV = 260 nm |
| wavelength | |
| Temperature of | 35 °C |
| column oven | |
| Flow rate | 0.9 mL/min. |
| Injection volume | 5 μL |
| Analysis Time | 30 min. |
| Retention Time | RT = 10.2 min (compound 12 enantiomer), |
| | RT = 12.1 min (compound 12) |

The following abbreviations may be used herein.
Me: methyl
Et: ethyl
tBu: tert-butyl
Ph: phenyl
DMF: N,N-dimethylformamide
TFA: trifluoroacetic acid
THF: tetrahydrofuran
DMSO: dimethylsulfoxide
DCM: dichloromethane
MTBE: methyl-tert-butyl ether
TCCA: trichloroisocyanuric acid
TEMPO: 2,2,6,6-tetramethylpiperidine 1-oxyl
WSC: 1-(3-dimethylaminopropyl)-3-ethylcarbodiimide
HOBt: 1-hydroxybenzotriazole
wt %: weight %
v/w: volume/weight, which denotes the ratio of volume for weight of base material
RT: Retention Time
RRT: Relative Retention Time

The symbols used in NMR are s for singlet, d for doublet, t for triplet, q for quartet, and m for multiplet.

Room temperature means 10 °C to 30 °C.

### EXAMPLES

Next, the present invention is illustrated in more detail with reference to Examples and Reference examples below, but should not be limited thereto. The following things may be changed without departing from the scope of the present disclosure. The compound names given in Examples and Reference examples below do not necessarily follow the IUPAC nomenclature.

### Example 1: Preparation of ethyl (R,E)-2-((1-phenylethyl)imino)acetate

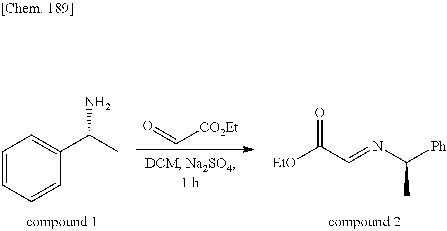

(R)-1-Phenylethan-1-amine (compound 1, 3000 g) was dissolved in DCM (11925 g) under nitrogen atmosphere. Na₂SO₄ (3000 g) was added to the solution. The suspension mixture was cooled to −20 to −10 °C. Ethyl 2-oxoacetate (5160 g, 50% solution in toluene) was dripped into the suspension at −20 to −10 °C. The mixture was warmed up to 15 to 25 °C and stirred for 2 to 4 h in a full conversion. The reaction mixture was filtered and the obtained cake was washed with DCM (6000 g). The filtrate (compound 2, a DCM solution regarded as 100% yield and the calculated amount of compound 2: 5080 g) was used for the next step directly.

### Example 1A: Preparation of ethyl (R,E)-2-((1-phenylethyl)imino)acetate

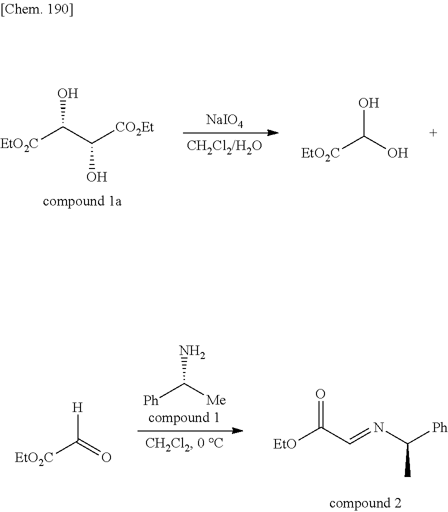

A solution of compound 1a (13.37 g, 64.8 mmol) in CH₂Cl₂ (105 mL) was prepared in a 125 mL Erlenmeyer flask, and then transferred to a 1000 mL three-neck flask equipped with overhead stirrer. Water (7.5 mL) was charged, and upon vigorous stirring, NaIO₄ (18.25 g, 85.3 mmol, 1.32 equiv) was charged in small portions. The resulting suspension was vigorously stirred at ambient temperature for 18 hr. The precipitate was then removed via vacuum filtration. The reaction flask was rinsed with CH₂Cl₂ (20 mL) and the washing liquid was filtered.

The combined filtrate (entry 1) was cooled to 0 °C and compound 1 (9 mL, 69.8 mmol) was added dropwise to the filtrate. The resulting cloudy mixture was stirred at 0 °C for 1 hr. The reaction mixture was treated with water (50 mL). The resulting mixture was transferred to a separatory funnel and the organic layer was separated. The aqueous layer was extracted with CH₂Cl₂ (25 mL). The organic layers were combined and concentrated in vacuo until the total volume of the solution was reduced to 75 mL. The resulting solution was dried over Na₂SO₄ and filtered into a 1000 mL three-neck flask fitted with an overhead stirrer and internal temperature probe. The filter cake was rinsed with CH₂Cl₂ (25 mL) and the filtrates were combined (entry 2).

### Example 2: Preparation of ethyl (1S,3S,4R)-2-((R)-1-phenylethyl)-2-azabicyclo[2.2.2]oct-5-ene-3-carboxylate

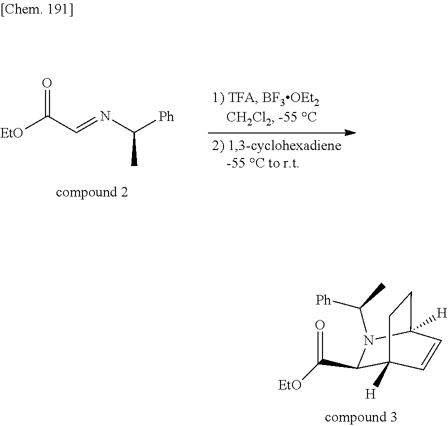 [SMILES: CCOC(=O)/C=N/[C@H](C)c1ccccc1 ; CCOC(=O)[C@@H]1[C@H]2C=C[C@H](CC2)N1[C@H](C)c1ccccc1]

The filtrate from the last step (containing 5.08 kg of ethyl (R,E)-2-((1-phenylethyl)imino)acetate, compound 2) was stirred and cooled to −60 to −50 °C under atmosphere. TFA (2.84 kg), BF₃·OEt₂ (effective content 3.50 kg, 7.46 kg of 47% solution), and 1,3-cyclohexadiene (2.6 kg) were added sequentially in dropwise at −60 to −50 °C into the reaction mixture. The reaction mixture was stirred for 4 h at −60 to −50 °C. Then the reaction mixture was warmed up to 10 to 20 °C and stirred for 10 to 15 h with full conversion. The reaction mixture was diluted with DCM (7.92 kg) and quenched with NaHCO₃ (37.24 kg, 10% aqueous solution). Then extra DCM (15.24 kg) was charged and the mixture was stirred. The lower organic phase was collected and concentrated to 4 to 6 v/v at 45 °C or lower temperature under vacuum. Then toluene (26.52 kg) was charged and the mixture was concentrated to 4 to 6 v/w at 60 °C under vacuum. Toluene (26.52 kg) was charged and the mixture was concentrated to 8-10 v/w and stirred at 55 to 60 °C for 1 h. The resulting mixture was cooled to 35 to 45 °C and stirred for 16 h to give a suspension. The mixture was filtered, and the wet cake was washed with MTBE (7.52 kg) to give a crude product as a white solid (a mixture of salts) containing 15% its isomer. The solid was dissolved in DCM (30.48 kg). NaHCO₃ (20.32 kg, 10% aqueous solution) was added in portions and the solution was adjusted to pH 8 to 9 at 15 to 25 °C and stirred for 1 h. The organic phase was collected and the aqueous phase was extracted with DCM (15.24 kg). The organic phases were combined and concentrated under vacuum to give crude compound 3 as a mixture of isomeric free bases.

### Purification

### Purification by Silica Gel Pad:

Crude compound 3 (free base 308 g) was dissolved in n-heptane (616 g) to give a clear solution. Silica gel (616 g) was charged in a Buchner funnel to give a silica gel padded. The crude compound 3/n-heptane solution was filtered through the silica gel pad. Then the pad was washed with n-heptane (8 kg) for eluting. The filtrate was concentrated to give compound 3 (243 g) in 78.9% recovery (yield) with 99% purity.

### Example 3: Preparation of ethyl (1S,3S,4S)-5-hydroxy-2-((R)-1-phenylethyl)-2-azabicyclo[2.2.2]octane-3-carboxylate

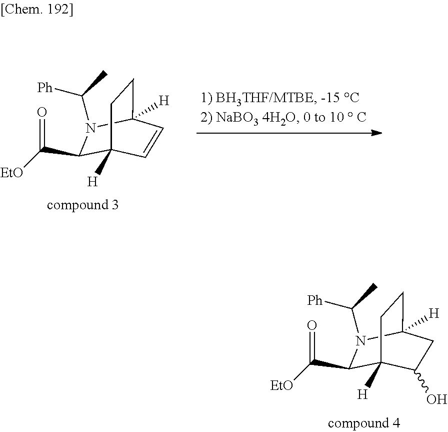 [SMILES: CCOC(=O)[C@@H]1[C@H]2C=C[C@H](CC2)N1[C@H](C)c1ccccc1 ; CCOC(=O)[C@@H]1[C@@H]2CC[C@@H](CC2O)N1[C@H](C)c1ccccc1]

Ethyl (1S,3S,4R)-2-((R)-1-phenylethyl)-2-azabicyclo[2.2.2]oct-5-ene-3-carboxylate (compound 3, 202 g) was dissolved in MTBE (299 g) under nitrogen atmosphere. The reaction mixture was cooled to −20 to −10 °C. BH₃THF (1 mol/L, 891 g) was dripped into the mixture at −20 to −10 °C. The mixture was stirred for 4 to 6 h then monitored by HPLC. Purified water (848 g) was dripped at no more than 10 °C. NaBO₃-4H₂O (327 g) was charged as solid in portion wise at 0 to 10 °C under agitation. The mixture was stirred at 0 to 10 °C for 14 to 18 h then monitored by HPLC. The mixture was quenched by adding sodium sulfite (20% aqueous solution, 892 g) at no more than 30 °C and diluted with ethyl acetate (455 g). The reaction mixture was filtered and the cake was washed with EtOAc (202 g). The filtrate was set for phase split. The upper organic phase was collected and dried with Na₂SO₄ (404 g) to purge the residual Na₂SO₄. The drying reagent was filtered off and the solid was washed with ethyl acetate (404 g). The filtrate was concentrated to 4 to 5 v/w at 45 °C under vacuum to give crude ethyl (1S,3S,4S)-5-hydroxy-2-((R)-1-phenylethyl)-2-azabicyclo[2.2.2]octane-3-carboxylate (compound 4) as a light-yellow solution. This crude product solution was regarded as 100% yield and used for the next step directly.

### Example 4: Preparation of ethyl (1S,3S,4S)-5-hydroxy-2-azabicyclo[2.2.2]octane-3-carboxylate

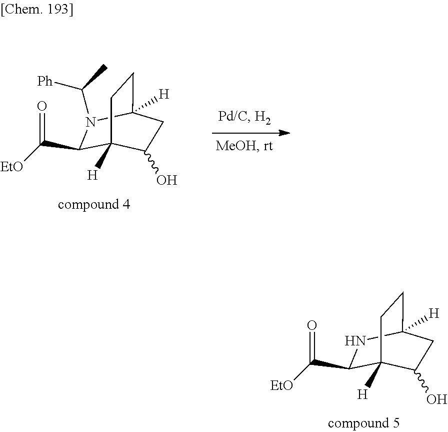 [SMILES: CCOC(=O)[C@@H]1[C@@H]2CC[C@@H](CC2O)N1[C@H](C)c1ccccc1 ; CCOC(=O)[C@H]1N[C@H]2CC[C@@H]1C(O)C2]

To a solution of ethyl (1S,3S,4S)-5-hydroxy-2-((R)-1-phenylethyl)-2-azabicyclo[2.2.2]octane-3-carboxylate (compound 4, 6.08 kg solution, containing 1.49 kg) was charged 10% Pd/C (0.19 kg, 65% water content) under inert condition. The reaction was conducted under 0.5 to 0.8 MPa hydrogen at 20 to 30 °C for 16 to 24 h. The reaction mixture was filtered through a pad of Celite (0.6 kg). The filter cake was washed with ethyl acetate (1.49 kg) and the filtrate was concentrated to give crude product 4. The crude product was regarded as 100% yield and used for the next step reaction directly.

### Example 5: Preparation of (1S,3S,4S)-2-(tert-butoxycarbonyl)-5-hydroxy-2-azabicyclo[2.2.2]octane-3-carboxylic acid

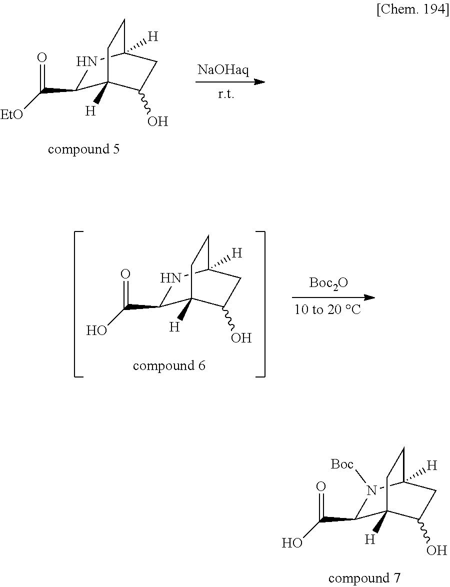

The ethyl (1S,3S,4S)-5-hydroxy-2-azabicyclo[2.2.2]octane-3-carboxylate (compound 5) from the last step was mixed with MTBE (464.7 g) and n-heptane (427.2 g). The resulting solution was extracted with purified water (1.05 kg) and the upper organic layer was removed as liquid waste. The lower aqueous phase, which contained compound 5, was mixed with 1,4-dioxane (1.05 kg) and cooled to 10 to 15 °C. Then 30% NaOH aqueous solution (154.13 g) was dripped into the reaction mixture at 0 to 20 °C. The reaction mixture was stirred at 10 to 20 °C for 3 to 4 h to give compound 6. (Boc)₂O (275.4 g) was diluted with 1,4-dioxane (105 g) and added to the above reaction mixture at 5 to 20 °C. The reaction mixture was stirred at 10 to 20 °C for 16 to 20 h and cooled to 0 to 10 °C. The pre-mixed solvents (EtOAc 810 g, MeOH 90 g) were added. 4.0 M HCl aqueous solution (351.25 g) was used to adjust pH to 1 to 3 at 0 to 10 °C. Solid NaCl (314.9 g) was charged. The organic layer was collected and the aqueous phase was extracted with mixture solvents (EtOAc 810 g, MeOH 90 g) three times. The organic phases were combined and dried over Na₂SO₄ (418.8 g). The mixture was filtered and the cake was washed with ethyl acetate (209.4 g). The filtrate was concentrated to 2 to 3 v/w. The concentrated mixture was stirred at 20 to 30 °C for 16 to 17 h and then cooled to 5 to 10 °C to give a suspension. The suspension was filtered and the cake was washed with n-heptane (284.78 g). The wet cake was dried and 116.9 g of compound 7 was obtained in 41.03% yield.

### Example 6: Preparation of (1S,3S,4S)-2-(tert-butoxycarbonyl)-5-oxo-2-azabicyclo[2.2.2]octane-3-carboxylic acid

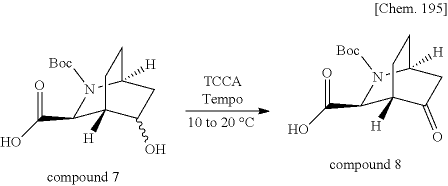 [SMILES: CC(C)(C)OC(=O)N1[C@H]2CC[C@H](C(O)C2)[C@H]1C(=O)O ; CC(C)(C)OC(=O)N1[C@H]2CC[C@H](C(=O)C2)[C@H]1C(=O)O]

(1S,3S,4S)-2-(tert-Butoxycarbonyl)-5-hydroxy-2-azabicyclo[2.2.2]octane-3-carboxylic acid (compound 7, 50 g) was suspended in a reaction vessel with EtOAc (500 g). The mixture was cooled to −5 to 5 °C. TEMPO (1.45 g) was charged in one portion followed by solid TCCA (32.1 g) portion wise at −5 to 5 °C. The reaction mixture was stirred at −5 to 5 °C for 4 to 6 h and resulted in a full conversion. The reaction mixture was diluted with EtOAc (1500 g) and the suspension was filtered through a pad of Celite (30 g). The cake was washed with EtOAc (100 g). The filtrate was quenched with FeSO₄ solution (102.5 g FeSO₄-7H₂O in 187 g purified water) at 0 to 10 °C. The aqueous phase was removed and the organic phase was dried over Na₂SO₄ (100 g) at 20 to 30 °C for 14 to 18 h. The reaction mixture was filtered and the cake was washed with EtOAc (100 g). The filtrate was concentrated to 2 to 3 v/w at 45 °C under vacuum to give a suspension. n-Heptane (204 g, 6 v/w) was charged to induce more precipitation. The mixture was filtered and the product cake was washed with n-heptane. The wet cake was dried at 35 to 45 °C under vacuum, and compound 8 (45.37 g) was obtained in 91.4% yield and 97.5 HPLC purity.

### Example 7A: Preparation of tert-butyl (1S,3S,4S)-3-[2-(tert-butoxycarbonyl)-2,7-diazaspiro[3.5]nonane-7-carbonyl]-5-oxo-2-azabicyclo[2.2.2]octane-2-carboxylate

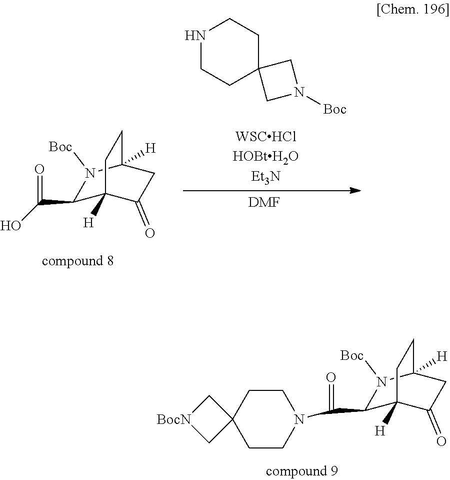

Compound 8 (20 g, 74.27 mmol) and HOBt H₂O (11.60 g, 1.02 eq.) were placed in 1 L 5-neck separable flask. DMF (60 mL, 3 v/w) was added to the mixture at 25±5 °C. The mixture was cooled down to 0±5 °C. WSC HCl (15.66 g, 1.1 eq.) in DMF (60 mL, 3 v/w) and water (10 mL, 0.5 v/w) was added to the mixture at 0±5 °C. The mixture was stirred for 1 hour at 0±5 °C tert-Butyl 2,7-diazaspiro[3.5]nonane-2-carboxylate (17.65 g, 1.1 eq.) in DMF (40 mL, 4 v/w) was added to the mixture at 0±5 °C. The reaction mixture was stirred for 2 hours at 0±5 °C. Et₃N (8.27 g, 1.1 eq.) was added to the mixture at 0±5 °C. The reaction mixture was stirred for more than 12 hours. Water (90 mL, 4.5 v/w) was added to the mixture at 0±5 °C. The mixture was stirred for 2 hours at 0±5 °C. The precipitate was collected by filtration and then rinsed with water (60 mL, 3 v/w) 3 times. The filtrate cake was dried under reduced pressure at 60±5 °C to give compound 9 (33.61 g) as a nearly white crystalline powder. Yield 94.8% (97.9 HPLC area %, analysis by Method 1).

1H-NMR (CDCl3) δ: 4.79 and 4.73 (total 1H, each m), 4.70 and 4.55 (total 1H, each m), 3.73-3.25 (total 8H, m), 2.67-2.29 (total 4H, m), 2.01-1.50 (total 7H, m) 1.50-1.41 (total 12H, m), 1.39 (6H, s).

### Example 7B: Preparation of tert-butyl (1S,3S,4S)-3-[2-(tert-butoxycarbonyl)-2,7-diazaspiro[3.5]nonane-7-carbonyl]-5-oxo-2-azabicyclo[2.2.2]octane-2-carboxylate

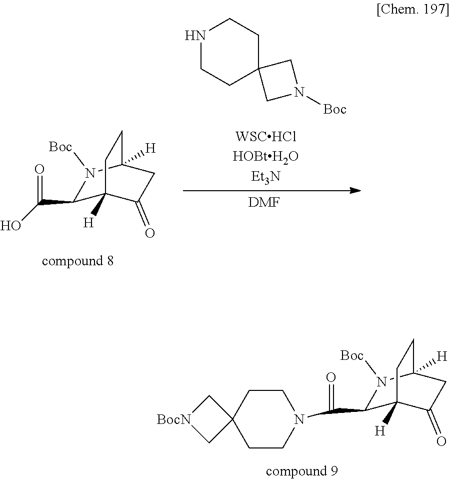

Compound 8 (1000 g, 3.71 mol) and HOBt H₂O (580 g, 1.02 eq.) were placed in 20 L separable flask. DMF (3 L, 3 v/w) was added to the mixture at 25±5 °C. The mixture was cooled down to 0±5 °C. WSC HCl (783 g, 1.1 eq.) in DMF (3 L, 3 v/w) and water (500 mL, 0.5 v/w) was added to the mixture at 0±5 °C. The mixture was stirred for 1 hour at 0±5 °C tert-Butyl 2,7-diazaspiro[3.5]nonane-2-carboxylate (882 g, 1.05 eq.) in DMF (4 L, 4 v/w) was added to the mixture at 0±5 °C. The reaction mixture was stirred for 2 hours at 0±5 °C. Et₃N (413 g, 1.1 eq.) was added to the mixture at 0±5 °C. The reaction mixture was stirred for 2 hours. The reaction mixture was warmed up to 25±5 °C and then stirred for more than 12 hours. Water (4.5 L, 4.5 v/w) was added to the mixture at 25±5 °C. The mixture was stirred for 2 hours at 25±5 °C. The precipitate was collected by filtration and then rinsed with water (3 L, 3 v/w) 3 times. The filtrate cake was dried under reduced pressure at 60±5 °C to give compound 9 (1695 g) as a nearly white crystalline powder. Yield 95.6% (98.7 HPLC area %, analysis by Method 1).

1H-NMR (CDCl3) δ: 4.79 and 4.73 (total 1H, each m), 4.70 and 4.55 (total 1H, each m), 3.73-3.25 (total 8H, m), 2.67-2.29 (total 4H, m), 2.01-1.50 (total 7H, m) 1.50-1.41 (total 12H, m), 1.39 (6H, s).

### Example 8A: Preparation of ((1S,3S,4R)-5-methylene-2-azabicyclo[2.2.2]octan-3-yl) (2,7-diazaspiro[3.5]nonan-7-yl)methanone dihydrochloride

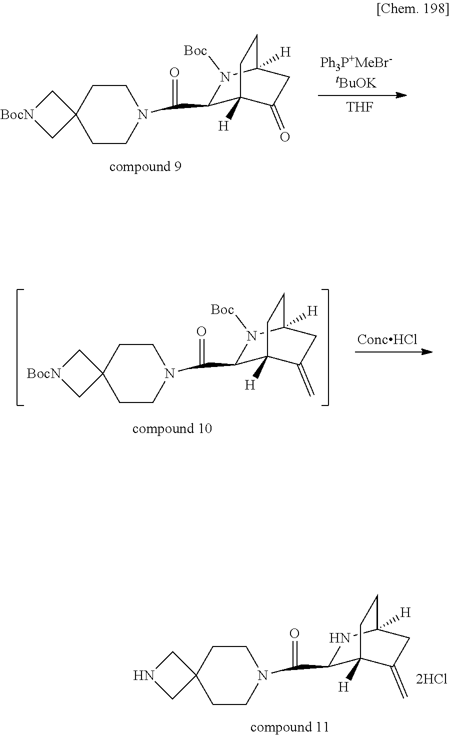

PPh₃MeBr (46.37 g, 1.55 eq.) was placed in 1 L 5-neck separable flask. THF (240 mL, 6 v/w) was charged into the flask at 20±5 °C. ^{t}BuOK 14.10 g (1.50 eq.) was added to the mixture at 20±5 °C. The mixture was stirred for 1 hour at 20±5 °C. Compound 9 (40 g, 83.752 mmol) in THF (240 mL, 6 v/w) was added to the mixture at 20±5 °C. The reaction mixture was stirred for 17.5 hours at 20±5 °C. Conc. HCl (48.8 mL, 7 eq.) was added to the mixture at 20±5 °C. The reaction mixture was warmed up to 40±5 °C. The reaction mixture was stirred for 6 hours at 40±5 °C. The reaction mixture was cooled down to 20±5 °C. The reaction mixture was stirred for 16 hours at 20±5 °C. The precipitate was collected by filtration and then rinsed with THF (120 mL, 3 v/w) 3 times. The filtrate cake was dried under reduced pressure at 50±5 °C to give compound 11 (36.66 g) as a nearly white crystalline powder. Yield 87.5%, determined by qNMR. 94.6 HPLC area %, analysis by Method 2.

¹H-NMR (D₂O) δ: 5.18 (1H, s), 5.04 (1H, s), 4.61 (1H, s), 4.02-3.93 (4H, m), 3.83 (1H, br-s), 3.70-3.62 (1H, m), 3.60-3.51 (1H, m), 3.50-3.44 (2H, m), 2.85-2.73 (2H, m), 2.68-2.58 (1H, m), 2.08-1.88 (5H, m), 1.81-1.69 (2H, m), 1.68-1.55 (1H, m).

### Example 8B: Preparation of ((1S,3S,4R)-5-methylene-2-azabicyclo[2.2.2]octan-3-yl) (2,7-diazaspiro[3.5]nonan-7-yl)methanone dihydrochloride

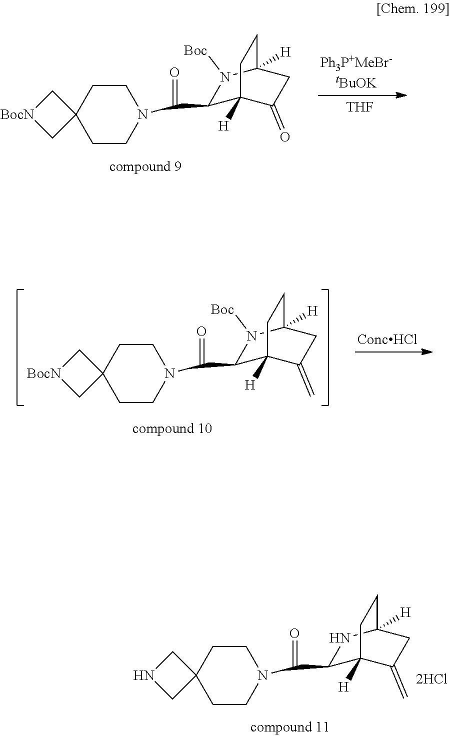

PPh₃MeBr (637 g, 1.55 eq.) was placed in 10 L 5-neck separable flask. THE (3.3 L, 6 v/w) was charged into the flask at 20±5 °C. tBuOK 194 g (1.50 eq.) was added to the mixture at 20±5 °C. The mixture was stirred for 1 hour at 20±5 °C. Compound 9 (550 g, 1.15 mol) in THF (3.3 L, 6 v/w) was added to the mixture at 20±5 °C. The reaction mixture was stirred for 18 hours at 20±5 °C. Conc. HCl (816 g, 7 eq.) was added to the mixture at 20±5 °C. The reaction mixture was warmed up to 40±5 °C. The reaction mixture was stirred for 6 hours at 40±5 °C. The reaction mixture was cooled down to 20±5 °C. The reaction mixture was stirred for 15 hours at 20±5 °C. The precipitate was collected by filtration and then rinsed with THE (2750 mL, 5 v/w) 3 times. The filtrate cake was dried under reduced pressure at 50±5 °C to give compound 11 (277 g) as a nearly white crystalline powder. Yield 87.3%, determined by qNMR. 86.4 HPLC area %, analysis by Method 2.

¹H-NMR (D₂O) δ: 5.18 (1H, s), 5.04 (1H, s), 4.61 (1H, s), 4.02-3.93 (4H, m), 3.83 (1H, br-s), 3.70-3.62 (1H, m), 3.60-3.51 (1H, m), 3.50-3.44 (2H, m), 2.85-2.73 (2H, m), 2.68-2.58 (1H, m), 2.08-1.88 (5H, m), 1.81-1.69 (2H, m), 1.68-1.55 (1H, m).

### Example 9A: Preparation of 5-fluoro-2-[(4-{7-[(1S,3S,4R)-5-methylidene-2-azabicyclo[2.2.2]octane-3-carbonyl]-2,7-diazaspiro[3.5]nonan-2-yl}pyrimidin-5-yl)oxy]-N,N-di(propan-2-yl)benzamide

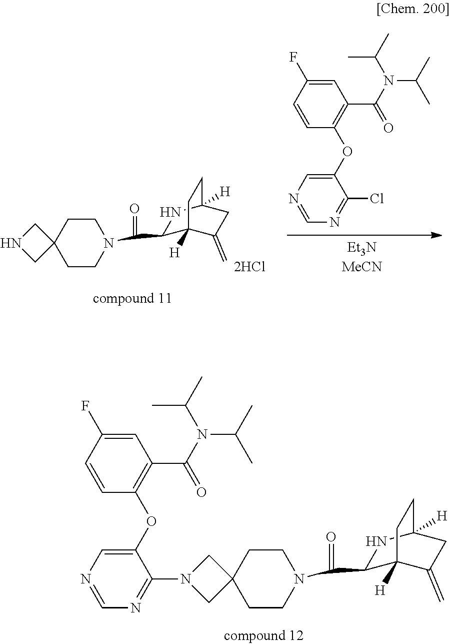 [SMILES: C=C1C[C@@H]2CC[C@H]1[C@@H](C(=O)N1CCC3(CC1)CNC3)N2 ; CC(C)N(C(=O)c1cc(F)ccc1Oc1cncnc1Cl)C(C)C ; C=C1C[C@@H]2CC[C@H]1[C@@H](C(=O)N1CCC3(CC1)CN(c1ncncc1Oc1ccc(F)cc1C(=O)N(C(C)C)C(C)C)C3)N2]

Compound 11 (6.27 g, 0.018 mol) was placed in 300 mL 5-neck separable flask. MeCN (40 mL, 8 v/w, based on salt free compound 11) was charged into the flask. The mixture was cooled down to 5±5 °C. Et₃N (15.0 mL, 6 eq.) was added to the mixture at 5±5 °C. The reaction mixture was stirred for 0.5 hours at 5±5 °C. 2-((4-Chloropyrimidin-5-yl)oxy]-5-fluoro-N,N-diisopropylbenzamide (6.01 g, 0.95 eq.) in MeCN (30 mL, 6 v/w) was added to the suspension at 5±5 °C. The reaction mixture was stirred for 23 hours at 5±5 °C. The precipitate was collected by filtration and then rinsed with cooling MeCN (20 mL, 4 v/w). The filtrate cake was filter dried at room temperature for more than 1 hour. The filtrate cake was washed with water (40 mL, 8 v/w) 3 times. The filtrate cake was dried under reduced pressure at 60±5 °C to give compound 12 (8.74 g) as a nearly white crystalline powder. Yield 82.2% (99.2 HPLC area %, analysis by Method 3). Compound 12 (8.0 g, 13.542 mmol) was placed in the 200 mL 5-neck separable flask. MeCN (30 mL, 3.75 v/w) and water (10 mL, 1.25 v/w) were charged into the flask. The mixture was warmed up to 60±5 °C. The mixture was stirred for 1 hour at 60±5 °C. Water (40 mL, 5 v/w) was added to the mixture at 60±5 °C. The mixture was stirred for 1 hour at 60±5 °C. Water (40 mL, 5 v/w) was added to the mixture at 60±5 °C. The mixture was stirred for 1 hour at 60±5 °C. The mixture was cooled down to 20±5 °C for 4 hours. The mixture was stirred for 12 hours at 20±5 °C. The precipitate was collected by filtration and then rinsed with water (16 mL, 2 v/w) 2 times. The filtrate cake was dried under reduced pressure at 50±5 °C to give compound 12 (7.63 g) as a nearly white crystalline powder. Yield 95.4% (99.8 HPLC area %, analysis by Method 3).

1H-NMR (CDCl3) δ: 8.28 (1H, s), 7.75 (1H, s), 7.23-7.19 (2H, m), 7.03-6.99 (1H, m) 4.36-4.24 (5H, m), 4.04-4.01 (2H, m), 3.67-3.50 (3H, m), 3.31 (4H, brs), 2.62 (1H, brs), 1.44-1.23 (4H, m), 1.43 (3H, d, J=6.7 Hz), 1.33 (3H, d, J=6.7 Hz), 1.03 (3H, d, J=6.0 Hz), 0.99 (3H, d, J=6.7 Hz), 0.67-0.61 (1H, m), 0.38-0.36 (2H, m), 0.01-0.00 (2H, m).

### Example 9B: Preparation of 5-fluoro-2-[(4-{7-[(1S,3S,4R)-5-methylidene-2-azabicyclo[2.2.2]octane-3-carbonyl]-2,7-diazaspiro[3.5]nonan-2-yl}pyrimidin-5-yl)oxy]-N,N-di(propan-2-yl)benzamide

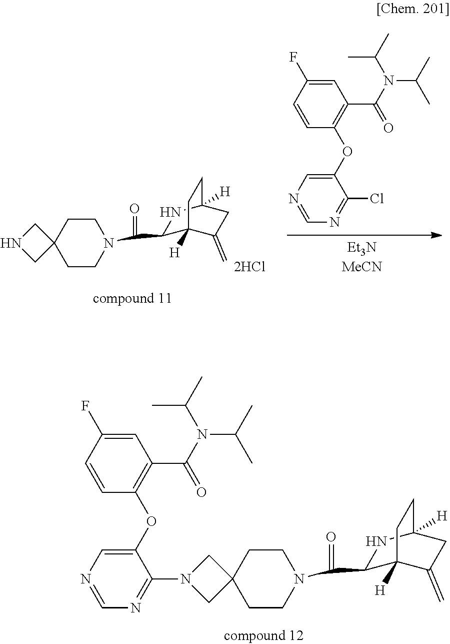 [SMILES: C=C1C[C@@H]2CC[C@H]1[C@@H](C(=O)N1CCC3(CC1)CNC3)N2 ; CC(C)N(C(=O)c1cc(F)ccc1Oc1cncnc1Cl)C(C)C ; C=C1C[C@@H]2CC[C@H]1[C@@H](C(=O)N1CCC3(CC1)CN(c1ncncc1Oc1ccc(F)cc1C(=O)N(C(C)C)C(C)C)C3)N2]

Compound 11 (233.5 g, 0.848 mol) was placed in 10 L separable flask. MeCN (1868 mL, 8 v/w, based on salt free compound 11) was charged into the flask. Et₃N (515 g, 6 eq.) was added to the mixture at 5±5 °C. The reaction mixture was stirred for 0.5 hours at 5±5 °C. The mixture was cooled down to 5±5 °C. 2-[(4-chloropyrimidin-5-yl)oxy]-5-fluoro-N,N-diisopropylbenzamide (283.4 g, 0.95 eq.) in MeCN (1400 mL, 6 v/w) was added to the suspension at 5±5 °C. The reaction mixture was stirred for 24 hours at 5±5 °C. The precipitate was collected by filtration and then rinsed with cooling MeCN (934 mL, 4 v/w). The filtrate cake was filter dried at room temperature for more than 1 hour. The filtrate cake was reslurried with water (3735 mL, 16 v/w) and then stirred for 3 hours. The precipitate was collected by filtration and then washed with water (1868 mL, 8 v/w) 2 times. The filtrate cake was dried under reduced pressure at 60±5 °C to give compound 12 (446 g) as a nearly white crystalline powder. Yield 89.1% (98.8 HPLC area %, analysis by Method 3).

Compound 12 (400 g, 0.677 mol) was placed in the 10 L separable flask. MeCN (1500 mL, 3.75 v/w) and water (500 mL, 1.25 v/w) were charged into the flask. The mixture was warmed up to 60±5 °C. The mixture was stirred for 3 hours at 60±5 °C. Water (1600 mL, 4 v/w) was added to the mixture at 60±5 °C. The mixture was stirred for 2 hours at 60±5 °C. The mixture was cooled down to 40±5 °C for 2 hours. The mixture was stirred for 2 hours at 40±5 °C. Water (2400 mL, 6 v/w) was added to the mixture at 40±5 °C. The mixture was stirred for 1 hour at 40±5 °C. The mixture was cooled down to 20±5 °C for 2 hours. The mixture was stirred for 11 hours at 20±5 °C. The precipitate was collected by filtration and then rinsed with water (800 mL, 2 v/w) 2 times. The filtrate cake was dried under reduced pressure at 60±5 °C to give compound 12 (347 g) as a nearly white crystalline powder. Yield 86.9% (99.6 HPLC area %, analysis by Method 3).

1H-NMR (CDCl3) δ: 8.28 (1H, s), 7.75 (1H, s), 7.23-7.19 (2H, m), 7.03-6.99 (1H, m) 4.36-4.24 (5H, m), 4.04-4.01 (2H, m), 3.67-3.50 (3H, m), 3.31 (4H, brs), 2.62 (1H, brs), 1.44-1.23 (4H, m), 1.43 (3H, d, J=6.7 Hz), 1.33 (3H, d, J=6.7 Hz), 1.03 (3H, d, J=6.0 Hz), 0.99 (3H, d, J=6.7 Hz), 0.67-0.61 (1H, m), 0.38-0.36 (2H, m), 0.01-0.00 (2H, m).

### Example 10A: Preparation of 5-fluoro-2-[(4-{7-[(1S,3S,4R)-5-methylidene-2-azabicyclo[2.2.2]octane-3-carbonyl]-2,7-diazaspiro[3.5]nonan-2-yl}pyrimidin-5-yl)oxy]-N,N-di(propan-2-yl)benzamide mono-L-tartrate

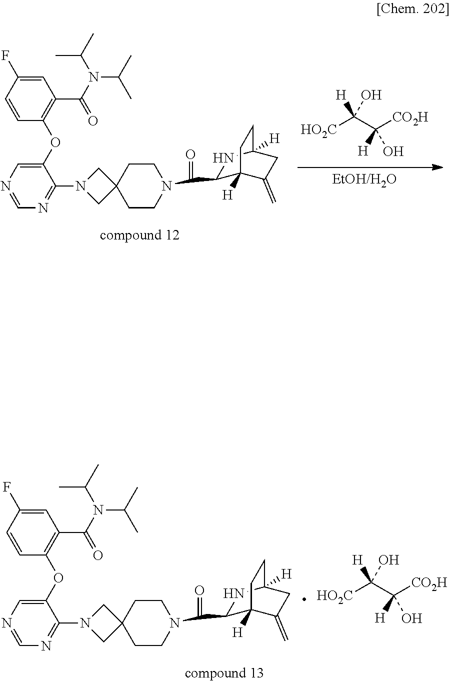 [SMILES: C=C1C[C@@H]2CC[C@H]1[C@@H](C(=O)N1CCC3(CC1)CN(c1ncncc1Oc1ccc(F)cc1C(=O)N(C(C)C)C(C)C)C3)N2 ; O=C(O)[C@H](O)[C@@H](O)C(=O)O ; C=C1C[C@@H]2CC[C@H]1[C@@H](C(=O)N1CCC3(CC1)CN(c1ncncc1Oc1ccc(F)cc1C(=O)N(C(C)C)C(C)C)C3)N2 ; O=C(O)[C@H](O)[C@@H](O)C(=O)O]

Compound 12 (3.0 g, 5.078 mmol) was placed in the 100 mL 4-neck round bottom flask. EtOH (19.5 mL, 6.5 v/w) was charged into the flask. The mixture was warmed up to 40±5 °C. The solution was cooled down to 25±5 °C. L-(+)-Tartaric acid (762 mg, 1.0 eq.) dissolved in EtOH (9.6 mL, 3.2 v/w) and water (0.9 mL, 0.3 v/w) was added to the mixture at 25±5 °C. The mixture was warmed up to 60±5 °C. Seed crystal was added to the mixture at 60±5 °C. The mixture was stirred for 2 hours at 60±5 °C. EtOH (15 mL, 5 v/w) was added to the mixture at 6 0±5 °C. The mixture was stirred for 2 hours at 60±5 °C. The suspension was cooled down to 0±5 °C. The mixture was stirred for 14 hours at 0±5 °C. The precipitate was collected by filtration and then rinsed with cooling EtOH (4.5 mL, 1.5 v/w) 2 times. The filtrate cake was dried under reduced pressure at 50±5 °C to give compound 13 (3.47 g) as a white crystalline powder. Yield 92.3% (99.9 HPLC area %, analysis by Method 3).

1H-NMR (DMSO-d6) δ: 8.28 (1H, s), 7.73 (1H, d, J=5.5 Hz), 7.26-7.17 (2H, m), 7.02 (1H, m) 5.08 (1H, s), 4.85 (1H, s), 4.32 (1H, s), 4.06-3.83 (total 6H, m), 3.68 (1H, m), 3.63-3.39 (total 4H, m), 3.32 (2H, br s), 2.66 (1H, m), 2.54 (1H, br s), 2.39 (1H, m), 1.91-1.64 (total, 5H, m), 1.60-1.47 (3H, m), 1.42 (3H, d, J=6.9 Hz), 1.33 (3H, d, J=6.4 Hz), 1.08 (3H, d, J=6.9 Hz), 0.98 (3H, d, J=6.4 Hz).

### Example 10B: Preparation of 5-fluoro-2-[(4-{7-[(1S,3S,4R)-5-methylidene-2-azabicyclo[2.2.2]octane-3-carbonyl]-2,7-diazaspiro[3.5]nonan-2-yl}pyrimidin-5-yl)oxy]-N,N-di(propan-2-yl)benzamide mono-L-tartrate

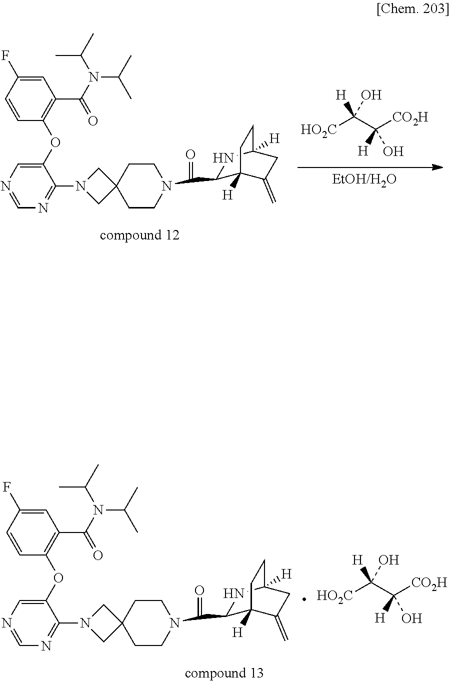 [SMILES: C=C1C[C@@H]2CC[C@H]1[C@@H](C(=O)N1CCC3(CC1)CN(c1ncncc1Oc1ccc(F)cc1C(=O)N(C(C)C)C(C)C)C3)N2 ; O=C(O)[C@H](O)[C@@H](O)C(=O)O ; C=C1C[C@@H]2CC[C@H]1[C@@H](C(=O)N1CCC3(CC1)CN(c1ncncc1Oc1ccc(F)cc1C(=O)N(C(C)C)C(C)C)C3)N2 ; O=C(O)[C@H](O)[C@@H](O)C(=O)O]

Compound 12 (10 g, 16.93 mmol) was placed in the 300 mL separable flask. EtOH (65 mL, 6.5 v/w) was charged into the flask. The mixture was warmed up to 60±5 °C. L-(+)-Tartaric acid (2.59 g, 1.02 eq.) dissolved in EtOH (32 mL, 3.2 v/w) and water (3 mL, 0.3 v/w) was added to the mixture at 60±5 °C. Seed crystal was added to the mixture at 60±5 °C. The mixture was stirred for 2 hours at 60±5 °C. EtOH (50 mL, 5 v/w) was added to the mixture at 60±5 °C. The mixture was stirred for 2 hours at 60±5 °C. The suspension was cooled down to 0±5 °C. The mixture was stirred for 14 hours at 0±5 °C. The precipitate was collected by filtration and then rinsed with cooling EtOH (15 mL, 1.5 v/w) 2 times. The filtrate cake was dried under reduced pressure at 50±5 °C to give compound 13 (11.79 g) as a white crystalline powder. Yield 94.0%

The Impurity profile is shown in Table 1: The purity of the target compound in analytical method 3 was 99.9%, with 0.05% of Compound A and 0.05% of Compound B as impurities. On the other hand, the purity of the target compound in analytical method 4 was 99.8%, with 0.06% of Compound C and <0.05% of the enantiomer. The relative retention time (RRT) refers to a relative ratio of the measured retention time to the retention time of the target compound (compound 12).

TABLE 1

| | Analytical | Amount | RT | | LC-MS |
|---|---|---|---|---|---|
| Impurity | Method | (%) | (min) | RRT | [M + H]⁺ |
| Compound A | Method 3 | 0.05 | 16.6 | 0.83 | 609.36 |
| Compound B | Method 3 | 0.05 | 36.2 | 1.82 | 906.48 |
| Compound C | Method 4 | 0.06 | 11.1 | 0.92 | 591.34 |

The amount of Compound A as determined by HPLC is preferably 0.15% or less, 0.14% or less, 0.13% or less, 0.12% or less, 0.11% or less, 0.10% or less, 0.09% or less, 0.08% or less, 0.07% or less, 0.06% or less, or 0.05% or less, more preferably includes 0.10% or less, 0.09% or less, 0.08% or less, 0.07% or less, 0.06% or less, or 0.05% or less, and most preferably 0.05% or less.

The amount of Compound B as determined by HPLC is preferably 0.15% or less, 0.14% or less, 0.13% or less, 0.12% or less, 0.11% or less, 0.10% or less, 0.09% or less, 0.08% or less, 0.07% or less, 0.06% or less, or 0.05% or less, more preferably 0.10% or less, 0.09% or less, 0.08% or less, 0.07% or less, 0.06% or less, or 0.05% or less, and most preferably 0.05% or less.

The amount of Compound C as determined by HPLC is preferably 0.15% or less, 0.14% or less, 0.13% or less, 0.12% or less, 0.11% or less, 0.10% or less, 0.09% or less, 0.08% or less, 0.07% or less, or 0.06% or less, more preferably 0.10% or less, 0.09% or less, 0.08% or less, 0.07% or less, or 0.06% or less, and most preferably 0.06% or less.

The structural formulas and NMR data (DMSO-d6) of the impurities Compound A, Compound B, and Compound C are as set forth below.

Compound A: 5-fluoro-2-[(4-{7-[(1S,3S,4S)-5-hydroxy-5-methyl-2-azabicyclo[2.2.2]octane-3-carbonyl-2,7-diazaspiro[3.5]nonan-2-yl}pyrimidin-5-yl)oxy]l-N,N-di(propan-2-yl)benzamide

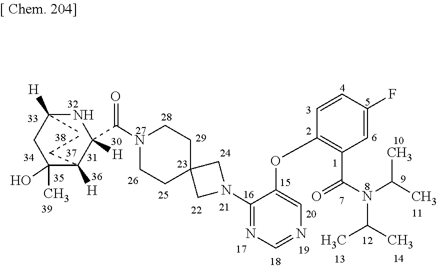

TABLE 2

| | Multiplicity | Relative | |
|---|---|---|---|
| Chemical Shift | (Coupling Constant | Intensity | |
| (ppm) | (Hz)) | ratio | Assignment |
| 0.99-1.01 | m | 3H | 10, 11, 13 or 14-H |
| 1.08 | d (J = 6.6) | 3H | 10, 11, 13 or 14-H |
| 1.25 | s | 3H | 39-H |
| 1.33 | d (J = 6.8) | 3H | 10, 11, 13 or 14-H |
| 1.43 | d (J = 6.5) | 3H | 10, 11, 13 or 14-H |
| 1.46-1.73 | m | 11H | 25, 29, 34, 36, 37 |
| | | | and 38-H |
| 2.83 | m | 1H | 33-H |
| 3.30-3.70 | m | 6H | 9, 12, 26, and 28-H |
| 3.86-3.96 | m | 4H | 22 and 24-H |
| 4.31, 4.63 | m | 1H | 31-H |
| 7.02-7.08 | m | 1H | 3-H |
| 7.20-7.25 | m | 2H | 4 and 6-H |
| 7.73 | s | 1H | 20-H |
| 7.74 | s | | |
| 8.27 | s | 1H | 18-H |
| 8.28 | s | | |

Compound B: 2-[(4-{(1S,3S,4R)-3-[2-(5-{2-[di(propan-2-yl)carbamoyl]-4-fluorophenoxy}pyrimidin-4-yl)-2,7-diazaspiro[3.5]nonane-7-carbonyl]-5-methylidene-2-azabicyclo[2.2.2]octan-2-yl}pyrimidin-5-yl)oxy]-5-fluoro-N,N-di(propan-2-yl)benzamide

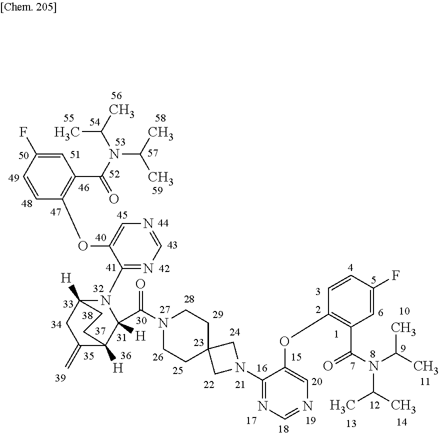 [SMILES: C=C1C[C@H]2CC[C@@H]1[C@@H](C(=O)N1CCC3(CC1)CN(c1ncncc1Oc1ccc(F)cc1C(=O)N(C(C)C)C(C)C)C3)N2c1ncncc1Oc1ccc(F)cc1C(=O)N(C(C)C)C(C)C]

TABLE 3

| | Multiplicity | Relative | |
|---|---|---|---|
| Chemical Shift | (Coupling Constant | intensity | |
| (ppm) | (Hz)) | ratio | Assignment |
| 1.04-1.47 | m | 24H | 10, 11, 13, 14, 55, |
| | | | 56, 58 and 59-H |
| 1.62-2.67 | m | 11H | 25, 29, 34, 36, 37 |
| | | | and 38-H |
| 3.20-3.96 | m | 12H | 9, 12, 22, 24, 26, |
| | | | 28, 54 and 57-H |
| 4.72-5.43 | m | 4H | 31, 33 and 39-H |
| 6.84-7.25 | m | 6H | 3, 4, 6, 48, 49 and |
| | | | 51-H |
| 7.62-7.87 | m | 2H | 20 and 45-H |
| 8.23-8.31 | m | 2H | 18 and 43-H |

Compound C: 5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide

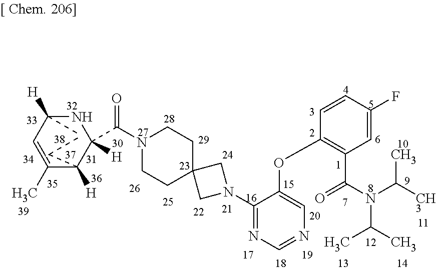

TABLE 4

| | Multiplicity | Relative | |
|---|---|---|---|
| Chemical Shift | (Coupling Constant | Intensity | |
| (ppm) | (Hz)) | ratio | Assignment |
| 1.00 | d (J = 6.3) | 3H | 10, 11, 13 or 14-H |
| 1.10 | d (J = 6.8) | 3H | 10, 11, 13 or 14-H |
| 1.34 | d (J = 6.5) | 3H | 10, 11, 13 or 14-H |
| 1.44 | d (J = 6.6) | 3H | 10, 11, 13 or 14-H |
| 1.46-1.73 | m | 9H | 25, 29, 36, 37 and |
| | | | 38-H |
| 1.86 | s | 3H | 39-H |
| 2.41 | m | 1H | 33-H |
| 3.30-3.71 | m | 6H | 9, 12, 26, and 28-H |
| 3.87-3.97 | m | 5H | 22, 24 and 31-H |
| 5.99 | m | 1H | 34-H |
| 7.02-7.06 | m | 1H | 3-H |
| 7.21-7.25 | m | 2H | 4 and 6-H |
| 7.74 | s | 1H | 20-H |
| 7.75 | s | | |
| 8.28 | s | 1H | 18-H |

Compound 13 (12.6 g, 17.01 mmol) was placed in the 300 mL 5-neck separable flask. EtOH (50 mL, 4 v/w) and water (8.1 mL, 0.65 v/w) was charged into the flask. The mixture was warmed up to 60±5 °C. EtOH (92 mL, 7.4 v/w) was added to the mixture at 60±5 °C. Seed crystal was added to the mixture at 60±5 °C. The mixture was stirred for 2 hours at 60±5 °C. The suspension was cooled down to 0±5 °C. The mixture was stirred for 14 hours at 0±5 °C. The precipitate was collected by filtration and then rinsed with cooling EtOH (15 mL, 1.2 v/w) twice. The filtrate cake was dried under reduced pressure at 50±5 °C to give compound 13 (11.1 g) as a white crystalline powder. Yield 88.2% (99.9 HPLC area %, analysis by Method 3, 100% ee, analysis by Method 4). 1H-NMR (DMSO-d6) δ: 8.28 (1H, s), 7.73 (1H, d, J=5.5 Hz), 7.26-7.17 (2H, m), 7.02 (1H, m) 5.08 (1H, s), 4.85 (1H, s), 4.32 (1H, s), 4.06-3.83 (total 6H, m), 3.68 (1H, m), 3.63-3.39 (total 4H, m), 3.32 (2H, br s), 2.66 (1H, m), 2.54 (1H, br s), 2.39 (1H, m), 1.91-1.64 (total, 5H, m), 1.60-1.47 (3H, m), 1.42 (3H, d, J=6.9 Hz), 1.33 (3H, d, J=6.4 Hz), 1.08 (3H, d, J=6.9 Hz), 0.98 (3H, d, J=6.4 Hz).

### Example 11: Preparation of ethyl (1S,3S,4S)-5-oxo-2-((R)-2-phenylethyl)-2-azabicyclo[2.2.2]octane-3-carboxylate

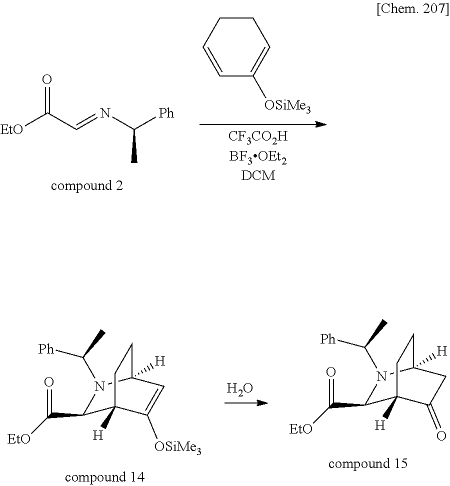

To a solution of compound 2 (1.17 g, assume 4.87 mmol) in dichloroethane (5 ml) at −78 °C, TFA (0.38 ml, 4.97 mmol) and BF₃-OEt₂ (0.62 ml, 4.97 mmol) were added, and the mixture was stirred for 15 minutes. 2-(Trimethylsilyloxy)-1,3-cyclohexadiene (940 mg, 5.34 mmol) was added (0.25 ml dichloromethane×2 was used for wash; it was added slowly to keep the temperature below −50 °C); the mixture was stirred for two hours, then allowed to warm naturally (over about 30 minutes); at 0 °C water (1 ml) was added, and stirring was continued for 15 minutes. The reaction mixture was added to NaHCO₃ aq. (25 ml) to neutralize, and extraction was performed using ethyl acetate (20 ml). The organic layer was washed with saturated aqueous sodium chloride solution and dried over anhydrous magnesium sulfate. The solvent was removed by vacuum distillation (and stored under refrigeration); the next day the concentrate was purified by silica gel column chromatography (30 g neutral silica; elution solvent was ethyl acetate/hexane at ratios of 1/19-1/3), and compound 15 (634 mg, 43.2%, pale yellow liquid, contained impurities) was obtained.

LC-MS; [M+H]⁺ 302.0

### Example 12: Preparation of 2-(tert-butyl) 3-ethyl (1S,3S,4S)-5-oxo-2-azabicyclo[2.2.2]octane-2,3-dicarboxylate

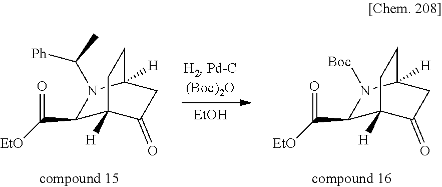 [SMILES: "EtOH" ; CCOC(=O)[C@@H]1[C@@H]2CC[C@@H](CC2=O)N1[C@H](C)c1ccccc1 ; CCOC(=O)[C@@H]1[C@@H]2CC[C@@H](CC2=O)N1C(=O)OC(C)(C)C]

To a solution of compound 15 (634 mg, 2.10 mmol) in ethanol (10 ml), (Boc)₂O (500 mg, 2.29 mmol), and 10% Pd—C (160 mg, 50% wet) were added, held under a hydrogen atmosphere, and stirred at room temperature for four hours. Then the mixture was filtered through Celite and washed with ethanol (3 ml×3). The solvent was removed by vacuum distillation, and the concentrate was purified by silica gel column chromatography (15 g neutral silica, elution solvent was ethyl acetate/hexane at ratios of 1/15-1/4), and compound 16 (306 mg, 49%, colorless, viscous liquid) was obtained.

LC-MS; [M+H]⁺ 298.0

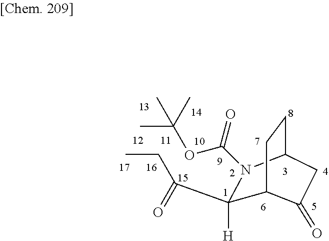 [SMILES: CCC(=O)C1C2CCC(CC2=O)N1C(=O)OC(C)(C)C]

| Position | Chemical shifts (δ_{H}), | |
|---|---|---|
| No. | Multiplicity, J(Hz) | Integration |
| 1 | 4.42, d, 3.2 | 1 |
| | 4.47, d, 3.2 | |
| 3 | 4.67, m | 1 |
| | 4.51, m | |
| 4 | 2.38, m | 2 |
| 5 | — | — |
| 6 | 2.83, m | 1 |
| | 2.75, m | |
| 7 | 1.77, m | 2 |
| 8 | 1.77, m, 1.91, m | 2 |
| 9 | — | — |
| 11 | — | — |
| 12, 13, 14 | 1.40, s | 9 |
| | 1.47, s | |
| 15 | — | — |
| 16 | 4.22, m | 2 |
| 17 | 1.31, m | 3 |

### Example 13: Preparation of (1S,3S,4S)-2-(tert-butoxycarbonyl)-5-oxo-2-azabicyclo[2.2.2]octane-3-carboxylic acid

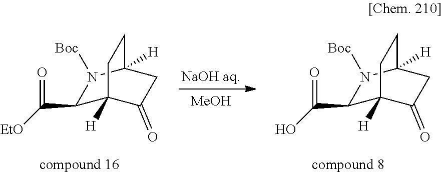 [SMILES: "MeOH" ; CCOC(=O)[C@@H]1[C@@H]2CC[C@@H](CC2=O)N1C(=O)OC(C)(C)C ; CC(C)(C)OC(=O)N1[C@H]2CC[C@H](C(=O)C2)[C@H]1C(=O)O]

To a solution of compound 16 (306 mg, 1.02 mmol) in methanol (11 mL), 5N NaOH aq. (1.0 ml) was added, and the mixture was stirred at 50 °C for five hours. The mixture was cooled to room temperature and neutralized by adding 2N HCl aq. Some solvent was removed by concentration, and extraction was performed with chloroform. The organic phase was dried over anhydrous magnesium sulfate. The solvent was evaporated by vacuum distillation, and the concentrate was purified by slica gel column chromatography (15 g neutral silica, elution solvent was chloroform/methanol at ratios of 1/0-12.5/1). Chloroform (1 ml) was added to the concentrate (203 mg). Hexane (4 ml) was added slowly, and the mixture was stirred for 30 minutes. The solids were filtered, washed with chloroform/hexane (1/4, 1 ml×2) and hexane (1 ml×2), and dried to obtain compound 8 (124 mg, 45%, white solid product)

LC-MS; [M−H]⁻ 298.0

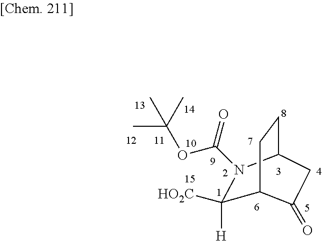 [SMILES: CC(C)(C)OC(=O)N1C2CCC(C(=O)C2)C1C(=O)O]

| Position | Chemical shifts | Chemical shifts (δ_{H}), | |
|---|---|---|---|
| No. | (δ_{C}) | Multiplicity, J(Hz) | Integration |
| 1 | 55.87 | 4.24, d, 4.2 | 1 |
| | 56.43 | 4.27, d, 4.2 | |
| 3 | 45.84 | 4.36, m | 1 |
| | 47.44 | 4.43, m | |
| 4 | 44.30 | 2.44, m | 2 |
| 5 | 209.89, 210.08 | — | — |
| 6 | 44.90 | 2.61, m | 1 |
| | 44.93 | 2.56, m | |
| 7 | 16.90 | 1.76, m | 2 |
| 8 | 24.22 | 1.76, m, 1.99, m | 2 |
| 9 | 152.62, 153.03 | — | — |
| 11 | 79.25, 79.44 | — | — |
| 12, 13, 14 | 27.73 | 1.34, s | 9 |
| | 27.95 | 1.40, s | |
| 15 | 170.41, 171.02 | — | — |
| COOH | — | 12.93, br s | 1 |

### Comparative Example 1

As Comparative Example 1, the processes disclosed in WO 2020/045334 can be summarized as follows in order to prepare the target compound of the present invention.

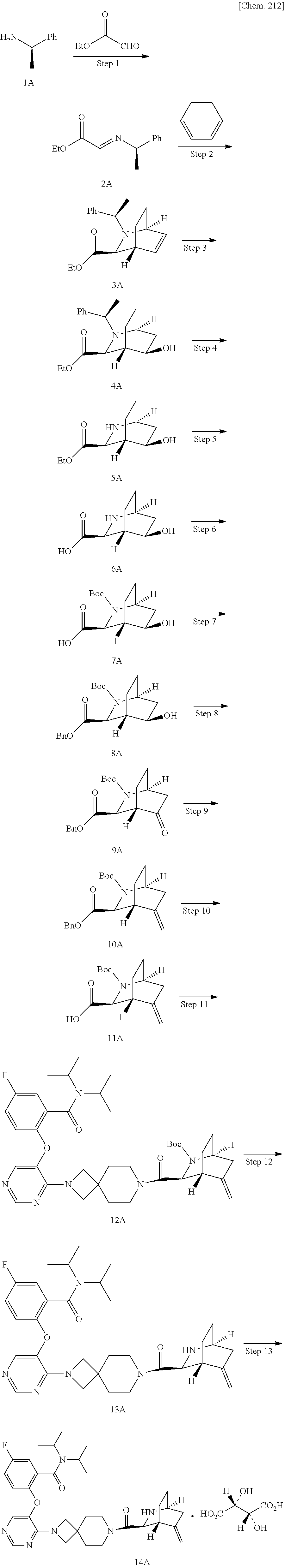

### 1. Comparison of Manufacturing Processes Between “Process 1 in the Present Invention” and “Comparative Example 1”

“Process 1” defined in the present specification is different from the above process of Comparative example 1, in that Process 1 of the present specification prepares compound (12) from compound (7) via 5 steps, while the process of Comparative example 1 prepares compound 13A (which corresponds to compound (12) in the present Process 1) from compound 7A (which corresponds to compound (7) in the present Process 1) via 6 steps. Specifically, the process of Comparative example 2 includes steps of protecting and deprotecting the carboxylic acid in Steps 7 and 10, which are unnecessary in Process 1 of the present disclosure. In addition, the present disclosure can produce compound (12) from compound (7) with an overall yield of 61%, whereas the overall yield of compound (13A) from compound (7A) in Comparative Example 1 is only 31%.

Thus, Process 1 of the present disclosure does not require additional protection and deprotection which are essential steps in Comparative example 1, and consequently the experimental operation in Process 1 of the present disclosure is simple because there are few liquid separation operations or purification operations using a silica gel column, and the overall yield is more than twice that of Comparative example 1. Therefore, Process 1 of the present disclosure makes it possible to produce the target compound with greater production of desired intermediate 12.

### 2. Comparison of Manufacturing Processes Between “Process 2 in the Present Invention” and “Comparative Example 1”

“Process 2” defined in the present specification is different from the above process of Comparative example 1, in that Process 2 of the present specification prepares compound (12) from compound (1) via 10 steps, while the process of Comparative example 1 prepares compound 13A (which corresponds to compound (12) in the present Process 2) from compound 1A (which corresponds to compound (1) in the present Process 2) via 12 steps. Specifically, in Process 2 of the present specification, compound (13) which has a trimethylsilyloxy group is prepared in Step M, and the trimethylsilyloxy group can be hydrolyzed to form carbonyl group, which can effectively prepare compound (14) without oxidation reaction. Using the process of the present disclosure, compound (12) can be produced from compound (1) with an overall yield of 7%, whereas the overall yield of compound (13A) from compound (1A) in Comparative Example 1 is only 3%.

Thus, Process 2 of the present disclosure does not require oxidation reaction which is essential steps in Comparative example 1, and consequently the experimental operation in Process 2 of the present disclosure is simple because there are few liquid separation operations or purification operations using a silica gel column, and the overall yield is more than twice that of Comparative example 1. Therefore, Process 2 of the present disclosure makes it possible to produce the target compound with a remarkable effect.

### Comparative Example 2

As Comparative Example 2 besides Comparative Example 1, the processes disclosed in WO 2020/045334 can be summarized as follows in order to prepare the target compound of the present invention.

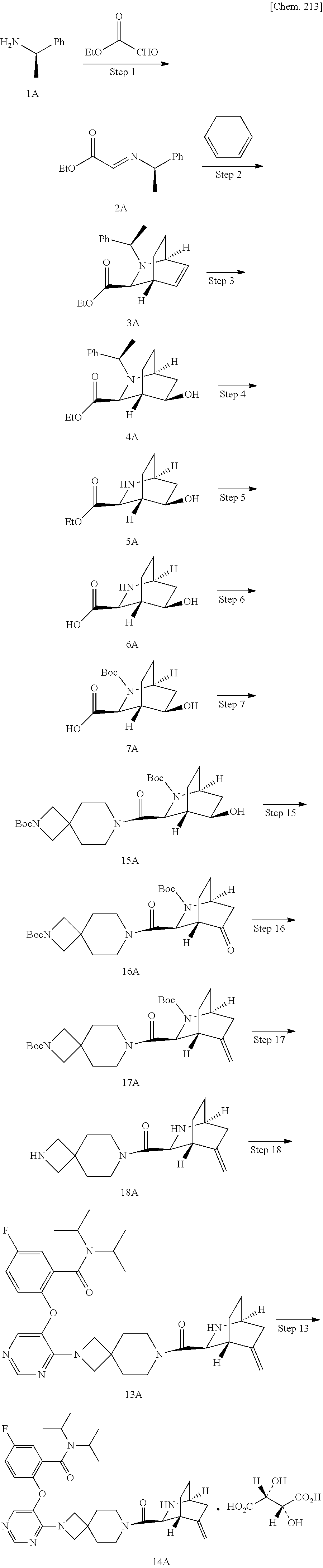

### 3. Comparison of Manufacturing Processes Between “Process 1 in the Present Invention” and “Comparative Example 2”

“Process 1” of the present disclosure and the process of Comparative example 2 have the same number of reaction steps from the starting materials. However, the process of Comparative example 2 requires alcohol oxidation using Swern oxidation, said oxidation reaction is not suitable as an industrial production method since it requires extremely low temperature conditions and generates dimethyl sulfide which gives offensive odor, and highly toxic carbon monoxide. On the other hand, Process 1 of the present disclosure uses TEMPO oxidation in Step G, said oxidation reaction is extremely desirable as an industrial production method since the oxidation can be performed at around room temperature and does not generate dimethyl sulfide or carbon monoxide, which is a problem in the process of Comparative example 2. Regarding the introduction of the diazaspiro-ring moiety contained in the target compound, Step 14 in the process of Comparative example 2 is carried out before oxidizing the hydroxyl group on the azabicyclo ring to a ketone, while Step H in Process 1 of the present invention is carried out after oxidizing the hydroxyl group on the azabicyclo ring to a ketone. The hydroxyl group here can cause a mixture of stereoisomers. Thus, by introducing a structurally-complex diazaspiro-ring moiety after such structurally-bothersome hydroxyl is oxygenized to a ketone, Process 1 of the present invention can reduce the complexity of its purification and analysis.

Thus, Process 1 of the present disclosure does not require industrially inappropriate oxidation reaction that poses a problem in the process of Comparative example 2, and can perform the alcohol oxidation under mild conditions. In addition, the experimental operation in Process 1 of the present disclosure is simple because there are few liquid separation operations or purification operations using a silica gel column. Further, by changing the timing of introduction of the diazaspiro-ring moiety, the complexity of purification and analysis can be reduced as compared with the process of Comparative example 2. Therefore, Process 1 of the present disclosure makes it possible to produce the target compound with a remarkable effect.

### 4. Comparison of Manufacturing Processes Between “Process 2 in the Present Invention” and “Comparative Example 2”

“Process 2” defined in the present specification is different from the above process of Comparative example 2, in that Process 2 of the present specification prepares compound (12) from compound (1) via 10 steps, while the process of Comparative example 2 prepares compound 13A (which corresponds to compound (12) in the present Process 2) from compound 1A (which corresponds to compound (1) in the present Process 2) via 11 steps. Specifically, in Process 2 of the present specification, compound (13) which has a trimethylsilyloxy group is prepared in Step M, and the trimethylsilyloxy group can be hydrolyzed to form carbonyl group, which can effectively prepare compound (14) without oxidation reaction. Using the process of the present disclosure, compound (12) can be produced from compound (1) with an overall yield of 7%, whereas the overall yield of compound (14A) from compound (1A) in Comparative Example 2 is only 5%.

In addition, in Process 2 of the present disclosure, the formula (13) has an enol ether, i.e., a stereoisomer resulting from a hydroxyl group moiety such as compound (7A) of Comparative example 2 does not exist in this portion. Thus, Process 1 of the present invention can reduce the complexity of its purification and analysis.

Thus, Process 2 of the present disclosure does not require oxidation reaction which is essential steps in Comparative example 2, and consequently the experimental operation in Process 2 of the present disclosure is simple because there are few liquid separation operations or purification operations using a silica gel column, the complexity of purification and analysis can be reduced, and the overall yield is more than twice that of Comparative example 2. Therefore, Process 2 of the present disclosure makes it possible to produce the target compound with a remarkable effect.

1. A mixture comprising 5-fluoro-2-[(4-{7-[(1S,3S,4R)-5-methylidene-2-azabicyclo[2.2.2]octane-3-carbonyl]-2,7-diazaspiro[3.5]nonan-2-yl}pyrimidin-5-yl)oxy]-N,N-di(propan-2-yl)benzamide or a pharmaceutically acceptable salt thereof, or a solvate thereof, and Compound A (5-fluoro-2-[(4-{7-[(1S,3S,4S)-5-hydroxy-5-methyl-2-azabicyclo[2.2.2]octane-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof.

2. The mixture of claim 1, wherein Compound A (5-fluoro-2-[(4-{7-[(1S,3S,4S)-5-hydroxy-5-methyl-2-azabicyclo[2.2.2]octane-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof is present in amount of about 0.15% or less of the mixture as determined by high performance liquid chromatography (HPLC).

3. The mixture of claim 1, wherein Compound A (5-fluoro-2-[(4-{7-[(1S,3S,4S)-5-hydroxy-5-methyl-2-azabicyclo[2.2.2]octane-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof is present in amount of about 0.1% or less of the mixture as determined by high performance liquid chromatography (HPLC).

4. The mixture of claim 1, wherein Compound A (5-fluoro-2-[(4-{7-[(1S,3S,4S)-5-hydroxy-5-methyl-2-azabicyclo[2.2.2]octane-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof is present in amount of about 0.05% or less of the mixture as determined by high performance liquid chromatography (HPLC).

5. The mixture according to claim 1, further comprising Compound B (2-[(4-{(1S,3S,4R)-3-[2-(5-{2-[di(propan-2-yl)carbamoyl]-4-fluorophenoxy}pyrimidin-4-yl)-2,7-diazaspiro[3.5]nonane-7-carbonyl]-5-methylidene-2-azabicyclo[2.2.2]octan-2-yl}pyrimidin-5-yl)oxy]-5-fluoro-N,N-di(propan-2-yl)benzamide) or a pharmaceutically acceptable salt thereof, or a solvate thereof.

6. The mixture according to claim 5, wherein Compound B (2-[(4-{(1S,3S,4R)-3-[2-(5-{2-[di(propan-2-yl)carbamoyl]-4-fluorophenoxy}pyrimidin-4-yl)-2,7-diazaspiro[3.5]nonane-7-carbonyl]-5-methylidene-2-azabicyclo[2.2.2]octan-2-yl}pyrimidin-5-yl)oxy]-5-fluoro-N,N-di(propan-2-yl)benzamide) or a pharmaceutically acceptable salt thereof, or a solvate thereof is present in amount of about 0.15% or less of the mixture as determined by high performance liquid chromatography (HPLC).

7. The mixture according to claim 5, wherein Compound B (2-[(4-{(1S,3S,4R)-3-[2-(5-{2-[di(propan-2-yl)carbamoyl]-4-fluorophenoxy}pyrimidin-4-yl)-2,7-diazaspiro[3.5]nonane-7-carbonyl]-5-methylidene-2-azabicyclo[2.2.2]octan-2-yl}pyrimidin-5-yl)oxy]-5-fluoro-N,N-di(propan-2-yl)benzamide) or a pharmaceutically acceptable salt thereof, or a solvate thereof is present in amount of about 0.1% or less of the mixture as determined by high performance liquid chromatography (HPLC).

8. The mixture according to claim 5, wherein Compound B (2-[(4-{(1S,3S,4R)-3-[2-(5-{2-[di(propan-2-yl)carbamoyl]-4-fluorophenoxy}pyrimidin-4-yl)-2,7-diazaspiro[3.5]nonane-7-carbonyl]-5-methylidene-2-azabicyclo[2.2.2]octan-2-yl}pyrimidin-5-yl)oxy]-5-fluoro-N,N-di(propan-2-yl)benzamide) or a pharmaceutically acceptable salt thereof, or a solvate thereof is present in amount of about 0.05% or less of the mixture as determined by high performance liquid chromatography (HPLC).

9. The mixture according to claim 5, further comprising Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof.

10. The mixture according to claim 9, wherein Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof is present in amount of about 0.15% or less of the mixture as determined by high performance liquid chromatography (HPLC).

11. The mixture according to claim 9, wherein Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof is present in amount of about 0.1% or less of the mixture as determined by high performance liquid chromatography (HPLC).

12. The mixture according to claim 9, wherein Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof is present in amount of about 0.06% or less of the mixture as determined by high performance liquid chromatography (HPLC).

13. The mixture according to claim 1, further comprising Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N, N-di(propan-2-yl)benzamide) or a pharmaceutically acceptable salt thereof, or a solvate thereof.

14. The mixture according to claim 13, wherein Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof is present in amount of about 0.15% or less of the mixture as determined by high performance liquid chromatography (HPLC).

15. The mixture according to claim 13, wherein Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof is present in amount of about 0.1% or less of the mixture as determined by high performance liquid chromatography (HPLC).

16. The mixture according to claim 13, wherein Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof is present in amount of about 0.06% or less of the mixture as determined by high performance liquid chromatography (HPLC).

17. A mixture comprising 5-fluoro-2-[(4-{7-[(1S,3S,4R)-5-methylidene-2-azabicyclo[2.2.2]octane-3-carbonyl]-2,7-diazaspiro[3.5]nonan-2-yl}pyrimidin-5-yl)oxy]-N,N-di(propan-2-yl)benzamide or a pharmaceutically acceptable salt thereof, or a solvate thereof, and Compound B (2-[(4-{(1S,3S,4R)-3-[2-(5-{2-[di(propan-2-yl)carbamoyl]-4-fluorophenoxy}pyrimidin-4-yl)-2,7-diazaspiro[3.5]nonane-7-carbonyl]-5-methylidene-2-azabicyclo[2.2.2]octan-2-yl}pyrimidin-5-yl)oxy]-5-fluoro-N,N-di(propan-2-yl)benzamide) or a pharmaceutically acceptable salt thereof, or a solvate thereof.

18. The mixture of claim 17, wherein Compound B (2-[(4-{(1S,3S,4R)-3-[2-(5-{2-[di(propan-2-yl)carbamoyl]-4-fluorophenoxy}pyrimidin-4-yl)-2,7-diazaspiro[3.5]nonane-7-carbonyl]-5-methylidene-2-azabicyclo[2.2.2]octan-2-yl}pyrimidin-5-yl)oxy]-5-fluoro-N,N-di(propan-2-yl)benzamide) or a pharmaceutically acceptable salt thereof, or a solvate thereof is present in amount of about 0.15% or less of the mixture as determined by high performance liquid chromatography (HPLC).

19. The mixture of claim 17, wherein Compound B (2-[(4-{(1S,3S,4R)-3-[2-(5-{2-[di(propan-2-yl)carbamoyl]-4-fluorophenoxy}pyrimidin-4-yl)-2,7-diazaspiro[3.5]nonane-7-carbonyl]-5-methylidene-2-azabicyclo[2.2.2]octan-2-yl}pyrimidin-5-yl)oxy]-5-fluoro-N,N-di(propan-2-yl)benzamide) or a pharmaceutically acceptable salt thereof, or a solvate thereof is present in amount of about 0.1% or less of the mixture as determined by high performance liquid chromatography (HPLC).

20. The mixture of claim 17, wherein Compound B (2-[(4-{(1S,3S,4R)-3-[2-(5-{2-[di(propan-2-yl)carbamoyl]-4-fluorophenoxy}pyrimidin-4-yl)-2,7-diazaspiro[3.5]nonane-7-carbonyl]-5-methylidene-2-azabicyclo[2.2.2]octan-2-yl}pyrimidin-5-yl)oxy]-5-fluoro-N, N-di(propan-2-yl)benzamide) or a pharmaceutically acceptable salt thereof, or a solvate thereof is present in amount of about 0.05% or less of the mixture as determined by high performance liquid chromatography (HPLC).

21. The mixture according to claim 17, further comprising Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof.

22. The mixture according to claim 21, wherein Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof is present in amount of about 0.15% or less of the mixture as determined by high performance liquid chromatography (HPLC).

23. The mixture according to claim 21, wherein Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof is present in amount of about 0.1% or less of the mixture as determined by high performance liquid chromatography (HPLC).

24. The mixture according to claim 21, wherein Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof is present in amount of about 0.06% or less of the mixture as determined by high performance liquid chromatography (HPLC).

25. A mixture comprising 5-fluoro-2-[(4-{7-[(1S,3S,4R)-5-methylidene-2-azabicyclo[2.2.2]octane-3-carbonyl]-2,7-diazaspiro[3.5]nonan-2-yl}pyrimidin-5-yl)oxy]-N,N-di(propan-2-yl)benzamide or a pharmaceutically acceptable salt thereof, or a solvate thereof, and Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof.

26. The mixture of claim 25, wherein Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof is present in amount of about 0.15% or less of the mixture as determined by high performance liquid chromatography (HPLC).

27. The mixture of claim 25, wherein Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof is present in amount of about 0.1% or less of the mixture as determined by high performance liquid chromatography (HPLC).

28. The mixture of claim 25, wherein Compound C (5-fluoro-2-[(4-{7-[(1S,3S,4R)-5-methyl-2-azabicyclo[2.2.2]oct-5-ene-3-carbonyl]-2,7-diazaspiro[3.5]nonan-2-yl}pyrimidin-5-yl)oxy]-N,N-di(propan-2-yl)benzamide) or a pharmaceutically acceptable salt thereof, or a solvate thereof is present in amount of about 0.06% or less of the mixture as determined by high performance liquid chromatography (HPLC).
